(19) United States
(12) Patent Application Publication
(10) Pub. No.: US 2019/0203207 A1
(43) Pub. Date: Jul. 4, 2019

(54) Anabolic Enhancers for Ameliorating Neurodegeneration

(71) Applicant: The Trustees of Columbia University in the City of New York, New York, NY (US)

(72) Inventor: Stephen TSANG, New York, NY (US)

(21) Appl. No.: 16/302,581

(22) PCT Filed: May 19, 2017
(86) PCT No.: PCT/US2017/033575

§ 371 Date: Nov. 16, 2018

Related U.S. Application Data
(60) Provisional application No. 62/339,241, filed on May 20, 2016.
(60) Provisional application No. 62/375,703, filed on Aug. 16, 2016.
(60) Provisional application No. 62/381,948, filed on Aug. 31, 2016.

(51) Int. Cl. C12N 15/113; A61P 27/02; A61P 25/28; C12N 7/00; A61K 9/00; C12N 15/11; C12N 9/22; A61K 45/06; A61K 31/7088; A61K 38/46; A61K 31/713

(52) U.S. Cl. CPC C12N 15/113; A61K 48/00; A61P 27/02; A61P 25/28; C12N 7/00; A61K 9/0048; C12N 15/11; C12N 9/22; A61K 45/06; A61K 31/7088; A61K 38/465; A61K 31/713; C12N 2310/111; C12N 2310/14; C12N 2310/122; C12N 2310/531; C12N 2750/14143; C12N 2320/32; C12N 2310/20; C12N 2800/80; C12N 15/1137

(57) ABSTRACT

The present disclosure relates to methods and compounds for promoting anabolic pathways in neuronal cells leading to improved neuronal survival. In particular, the present disclosure relates to inhibiting TSCI and or SIRT6 to promote glycolysis and neuronal survival in a variety of neurodegenerative conditions, and specifically in retinitis pigmentosa.

### CROSS-REFERENCE TO RELATED APPLICATIONS

The present application claims priority to U.S. Provisional Patent Application No. 62/339,241 filed. May 20, 2016, U.S. Provisional Application No. 62/375,703 filed Aug. 16, 2016, and U.S. Provisional Application No. 621381,948 filed Aug. 31, 2016, each of which is incorporated herein by reference in its entirety.

### STATEMENT OF GOVERNMENT SUPPORT

This invention was made with government support under grant 5P30CA013696 awarded by the National Cancer Institute. The government has certain rights in the invention.

### SEQUENCE LISTING

The instant application contains a Sequence Listing which has been filed to electronically in ASCII format and is hereby incorporated by reference in its entirety. Said ASCII copy, created on May 12, 2017, is named 01001-005178-WO0_SL.txt and is 2,402 bytes in size.

### FIELD OF THE INVENTION

The present disclosure relates to methods and compounds for promoting anabolic pathways in neuronal cells leading to improved neuronal survival. In particular, the present disclosure relates to inhibiting TSC1 and or SIRT6 to promote glycolysis and neuronal survival in a variety of neurodegenerative conditions, and specifically in retinitis pigmentosa.

### BACKGROUND

Retinitis pigmentosa (RP) is an incurable neurodegenerative condition that leads to progressive photoreceptor dysfunction, dysmorphosis and symptoms such as nyctalopia, tunnel vision and eventually, blindness (1-4). This disease is estimated to affect nearly 1 million people worldwide and leads to a substantial decrease in the ability of affected individuals to lead independent lives and conduct activities of daily living (1, 2). A heterogeneous genetic condition, RP is linked to more than 60 genes, most of which are exclusively expressed in rod photoreceptors (5-7). Due to the genetic diversity of RP any therapy that is gene specific can only benefit a small fraction of patients with RP. There is currently no effective therapeutic option for patients with RP or any other patient with a retinal degenerative disease, including atrophic age-related macular degeneration (AMD), which affects more than 1.5 million individuals in the United States (8).

Thus, there is an urgent need for additional therapeutics as well as more broadly effective gene therapies for alleviating, retinal degenerative diseases such as RP and AMD, and more broadly for promoting neuronal survival in neurodengenerative diseases such as glaucoma, Alzheimer's, Parkinson's, Huntington's, Amyotrophic lateral sclerosis (ALS), Lewy body dementia, and similar neurodegenerative conditions or other conditions that would benefit from upregulating anabolism and downregulating catabolism to promote neuronal survival.

### SUMMARY OF THE INVENTION

The methods of the present invention provide for increasing glycolysis in a neuronal cell comprising inhibiting TSC1 SIRT6, or a combination thereof and/or decreasing the level and/or activity of TSC1, SIRT6, or a combination thereof, in the neuronal cell.

Additional embodiments include a method of increasing, neuronal survival in patient(S) in need thereof, comprising altering glycosis by decreasing TSC1, SIRT6(3, or a combination thereof, in the neuronal cell.

Additional embodiments include a method of increasing photoreceptor survival comprising altering glycosis by inhibiting TSC1, SIRT6, or a combination thereof, and/or decreasing the level and/or activity of TSC1, SIRT6, or a combination thereof, in a photoreceptor cell.

The neuronal cell can be a cone cell, a rod cell, or a combination of cone cells, rod cells, and/or other retinal cells.

Altering glycosis can be accomplished by inhibiting TSC1, SIRT6, or a combination thereof, and/or decreasing the level and/or activity of TSC1, SIRT6, or a combination thereof, comprising administering an effective amount of an inhibitor selected from the group consisting of proteins, nucleic acids, chemicals and combinations thereof.

The nucleic acid can be selected from the group consisting of antisense oligonucleotide, siRNA, shRNA, gRNA and combinations thereof.

In certain embodiments, the decreasing comprises administering an effective amount of an inhibitor of TSC1, SIRT6, or a combination thereof.

In additional embodiments, the method comprises administering an effective amount of one or more SIRT6 inhibitors selected from the group consisting of: fenugreek seed extract, Vitexin (isolated from Hawthorn tree berries), quercetin, naringenin, vitexin, SYN17739303, BAS13555470, SYN 10366754, and BA S00417531.

In certain embodiments, the patient is suffering from one or more retinal degenerative diseases such retinitis pigmentose (RI)) age-related macular degeneration (AMD), or glaucoma, or one or more neurodegenerative diseases including Alzheimer's, Parkinson's, Huntington's, Amyotrophic lateral sclerosis (ALS), or Lewy body dementia.

Additional embodiments include a method of increasing photoreceptor survival in a patient in need thereof, comprising administering to the subject a therapeutically effective amount of:
a recombinant adeno-associated viral (AAV) vector encoding an inhibitor of Tsc1, Sirt6, or other metabolic reprogramming agent, or an inhibitor or activator of anabolism.

Additional embodiments include a method of increasing neuronal survival in patient(s) in need thereof, comprising administering a therapeutically effective amount of:
a recombinant adeno-associated viral (AAV) vector encoding an inhibitor of Tsc1, Sirt6, or other metabolic reprogramming agent, or an inhibitor or activator of anabolism, to at least one neuron in the patient.

In certain embodiments, the recombinant AAV vector is an AAV2 vector. In additional embodiments, the AAV vector is an AAV8 vector.

In yet additional embodiments, the AAV vectors are administered by intravitreal injection.

In yet additional embodiments, the AAV vectors are administered by subretinal injection.

Additional embodiments include a method of increasing photoreceptor survival in a patient in need thereof, comprising administering to the patient a therapeutically effective amount of:
(a) a fast recombinant adeno-associated viral (AAV) vector, wherein the first recombinant AAV comprises, (i) a first sequence(s) encoding at least one guide RNA that hybridizes to the endogenous Tsc1 or Sirt6 gene in the patient, and,
(b) a second recombinant AAV viral vector comprising a nucleic acid sequence encoding a Cas nuclease, wherein the Cas nuclease cleaves the TSC1 or Sirt6 gene creating a Tsc1 or Sirt6 knockout of the endogenous Tsc1 or Sirt6 gene in the patient.

Additional embodiments include a method of increasing neuronal survival in patient(s) in need thereof, comprising administering to the patient a therapeutically effective amount of:
(a) a first recombinant adeno-associated viral (AAV) vector, wherein the first recombinant AAV comprises, (i) a first sequences) encoding at least one guide RNA that hybridizes to the endogenous Tsc1 or Sir6 gene in the patient, and,
(b) a second recombinant AAV viral vector comprising a nucleic acid sequence encoding a Cas nuclease; wherein the Cas nuclease cleaves the Tsc1 or Sirt6 gene creating a Tsc1 or Sirt6 knockout of the endogenous Tsc1 or Sirt6 gene in the patient.

In additional embodiments, the invention relates to a method of increasing glycolysis in a neuronal cell in patient(s) in need thereof, comprising administering a therapeutically effective amount of: a recombinant adeno-associated viral (AAV) vector encoding an inhibitor of Tsc1, Sirt6: or other metabolic reprogramming agent, or an inhibitor or activator of anabolism, to at least one neuronal cell in the patient.

In additional embodiments, the invention relates to a method of increasing glycolysis in a neuronal cell in patient(s) in need thereof, comprising administering to the patient a therapeutically effective amount of:
(a) a first recombinant adeno-associated viral (AAV) vector, wherein the first recombinant AAV comprises, (i) a first sequence(s) encoding at least one guide RNA that hybridizes to the endogenous Tsc1 or Sirt6 gene in the patient, and,
(b) a second recombinant AAV viral vector comprising a nucleic acid sequence encoding a Cas nuclease; wherein the Cas nuclease cleaves the endogenous Tsc1 or Sirt6 gene creating a Tsc1 or Sirt6 knockout of the endogenous Tsc1 or Sirt6 gene in the patient's neuronal cell.

In certain embodiments, the recombinant AAV vector is an AAV2 vector. In additional embodiments, the AAV vector is an AAV8 vector. In certain embodiments, the Cas nuclease is Cas9.

In certain embodiments, the AAV vectors are administered by intravitreal injection. In additional embodiments, the AAV vectors are administered by subretinal injection.

In additional embodiments, any of the methods may further comprise administering an effective amount of one or more SIRT6 inhibitors selected from the group consisting of: fenugreek seed extract, Vitexin (isolated from Hawthorn tree berries), quereetin, naringenin, vitexin, SYN 17739303, BASI13555470, SYNI 10366754, and BAS00417531.

### BRIEF DESCRIPTION OF THE DRAWINGS

FIGS. 1A-F, ERG b-wave magnitudes and histology are comparable in Tsc1^{-/-} [symbol] and Tsc1^{loxP/laxP} [symbol] mice. FIGS. 1A-C are scotopic, mixed maximal, and photopic ERG recordings were acquired at five weeks post injection. Small grey dots represent data points from eyes; bold red dots represent means for the Tsc1^{-/-} [symbol] mice, and bold black dots represent means Tsc1^{loxP/loxP} [symbol] mice. To compare mean ERG outcomes or mean histological outcomes between the Tsc1^{-/-} [symbol] and Tsc1^{lox/P/loxP} [symbol] groups, linear mixed models with random intercepts for mice were fit. Average b-wave magnitudes between the groups had no statistically significant mean difference. ((A)P=0.80; (B)P=0.59; (C)P=0.89), FIGS. 1D-F are H&E staining of retinal sections also had comparable mean ONL and IS/OS layers between the groups. These values were quantified by measuring the thickness of the ONL and IS/OS, which were observed to be statistically insignificant. ((FIG. 1D) Yellow bar indicates ONL, white bar indicates IS/OS, scale bar=50 μm; (FIG. 1E) P=0.98 (FIG. 1F) P=0.78); for ERG, n=8 for Tsc1^{-/-} [symbol] ^{, n=}7 for Tsc1^{loxP/loxP} [symbol] ; for histology, n=7 for both groups.).

FIGS. 2A-B. Tsc1^{-/-} [symbol] have enhanced photoreceptor survival compared to Tsc1^{loxP/loxP} [symbol] controls for at least 3 weeks, FIG. 2A are H&E-stained retinal sections that were compared at 10, 17, and 24 days, ONL and IS/OS thickness were greater at every time point in the Tsc1^{-/-} [symbol] group compared to controls. Tamoxifen or oil injection occurred at P10. Histology was quantified statistically over time.

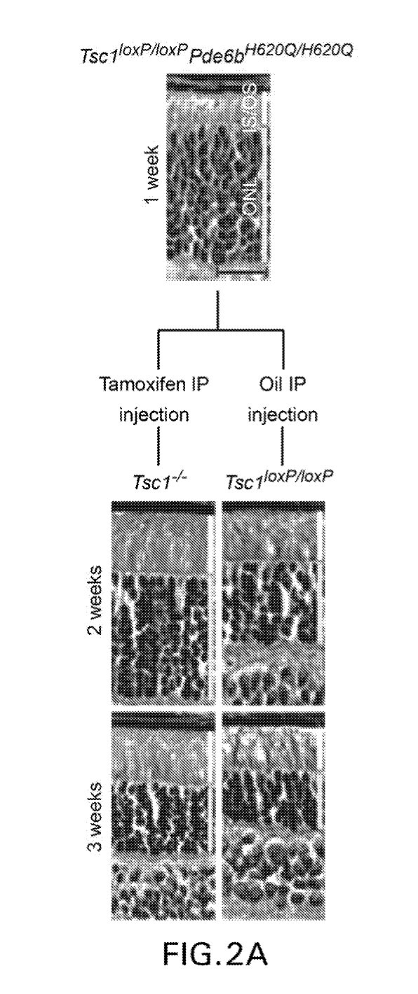

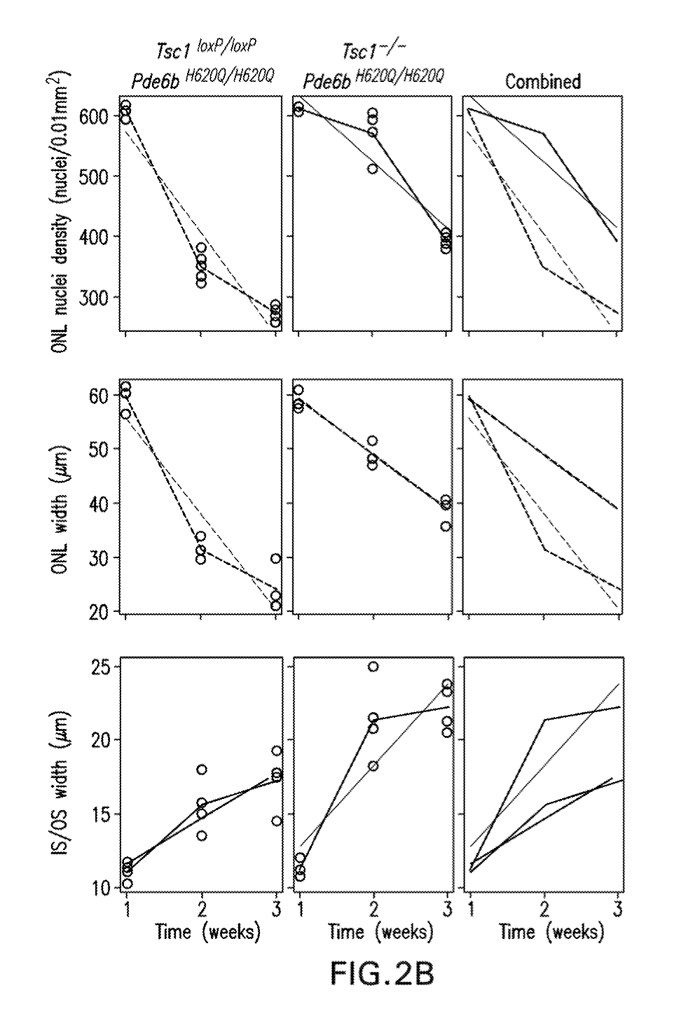

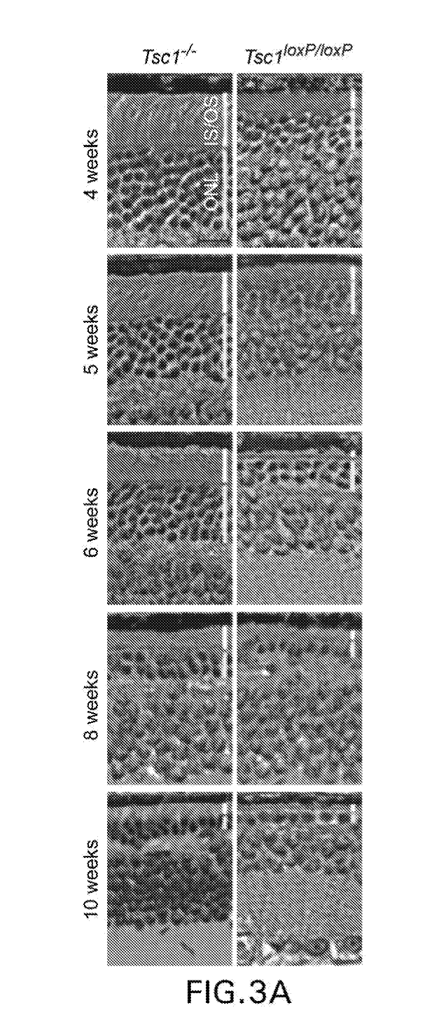

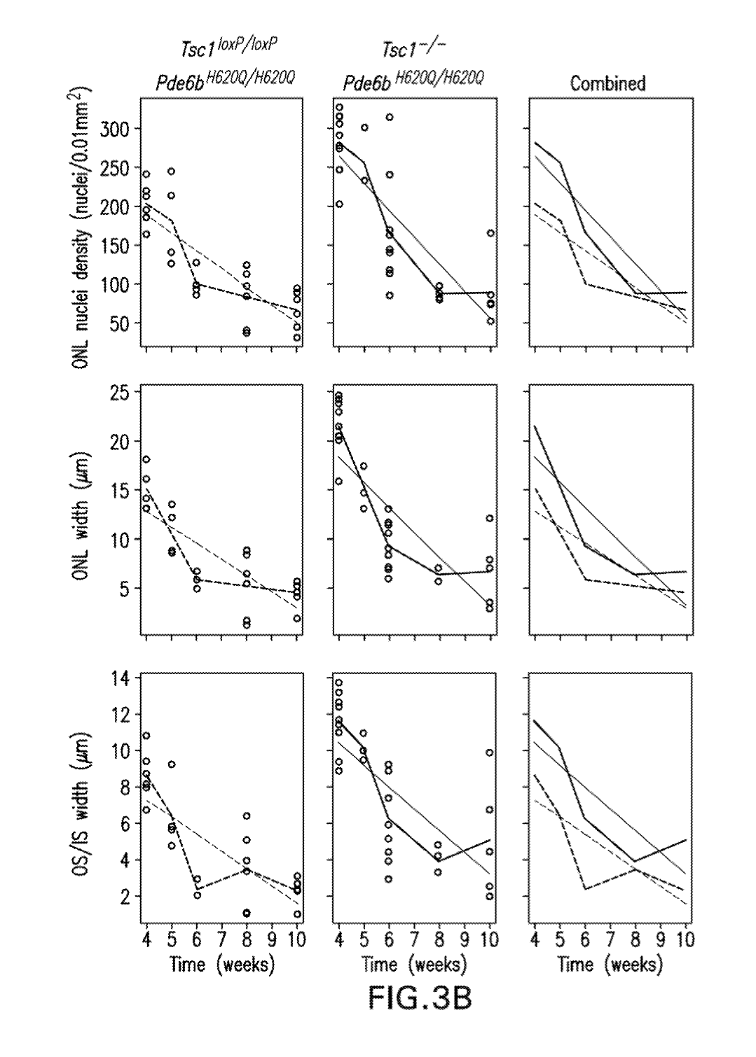

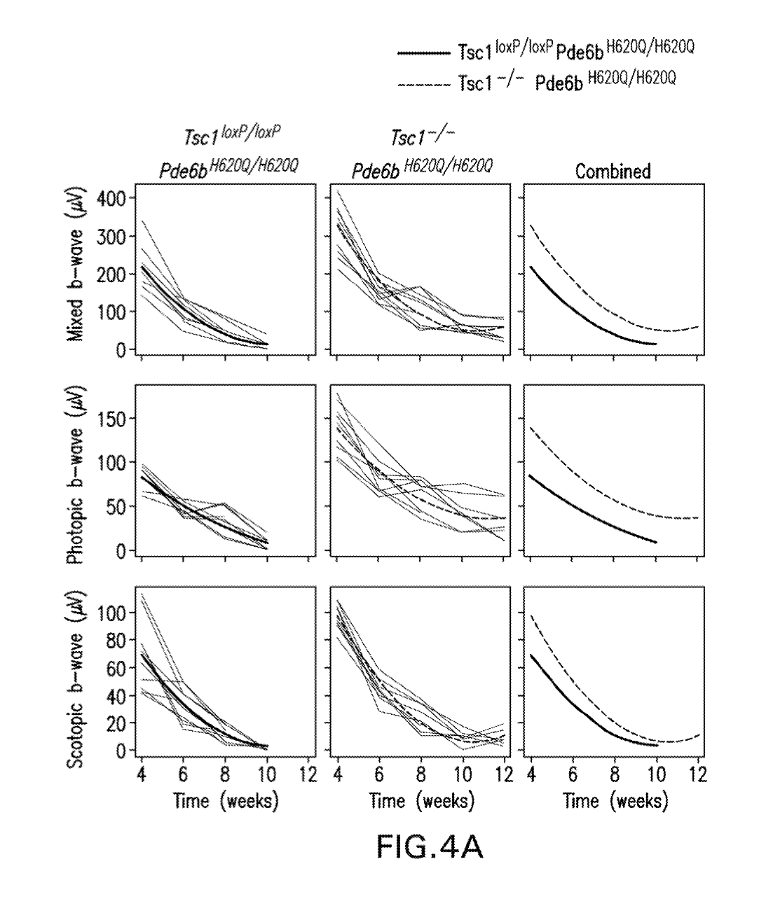

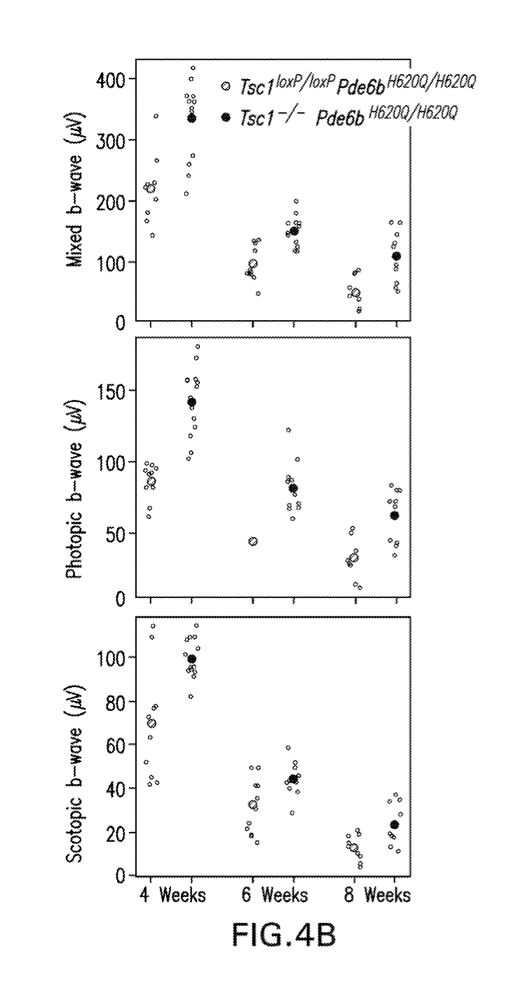

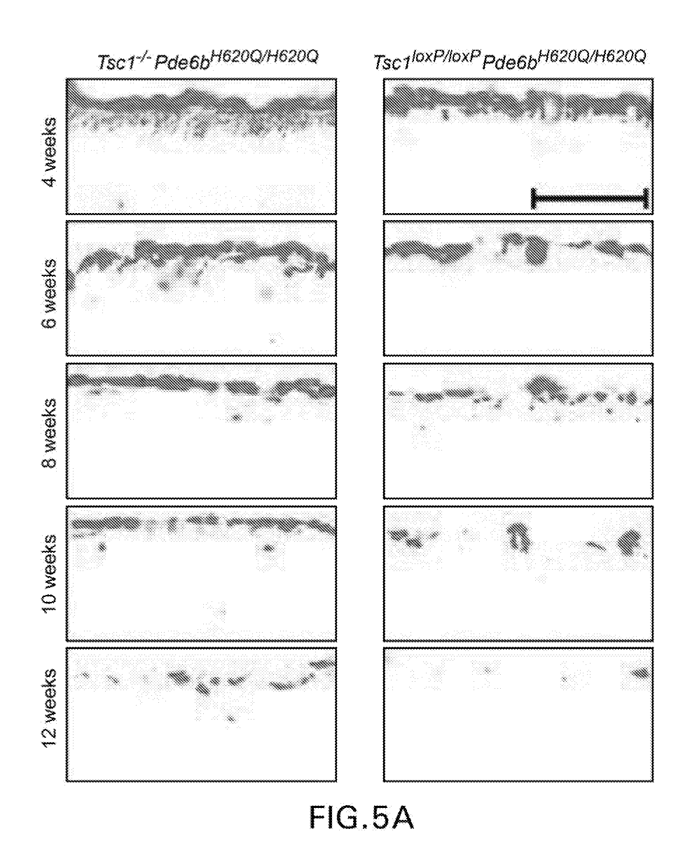

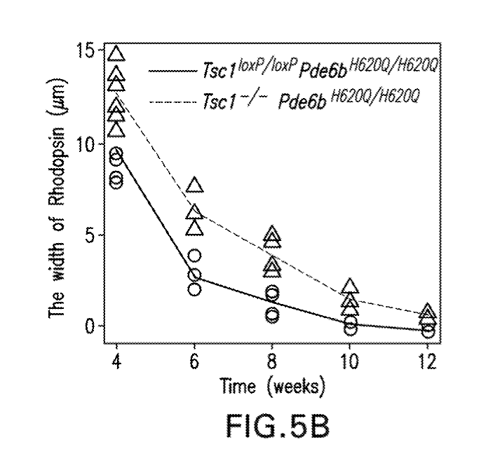

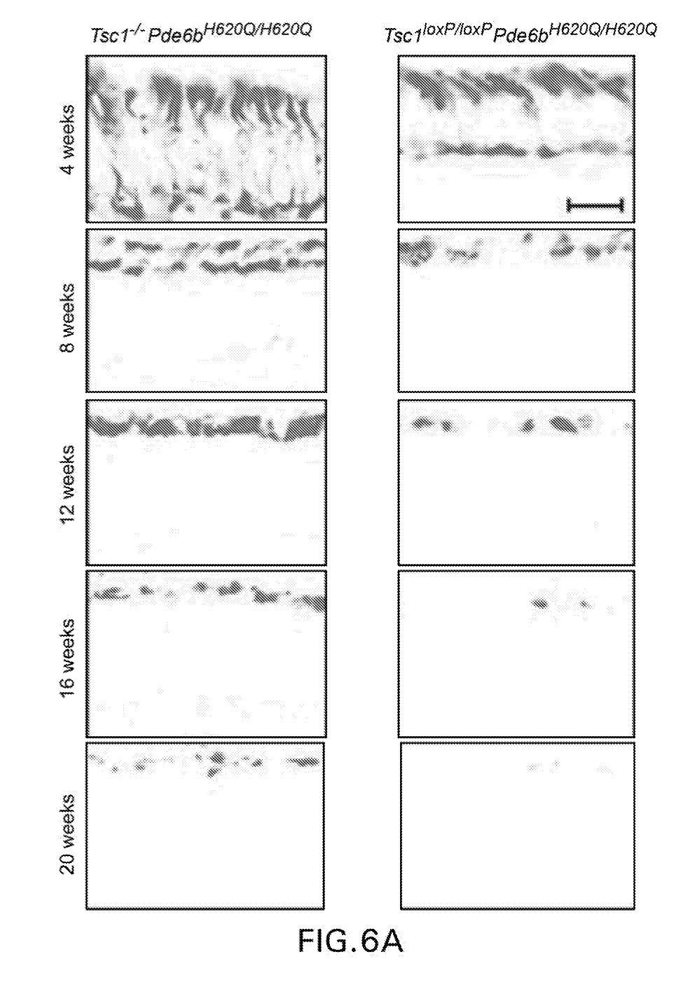

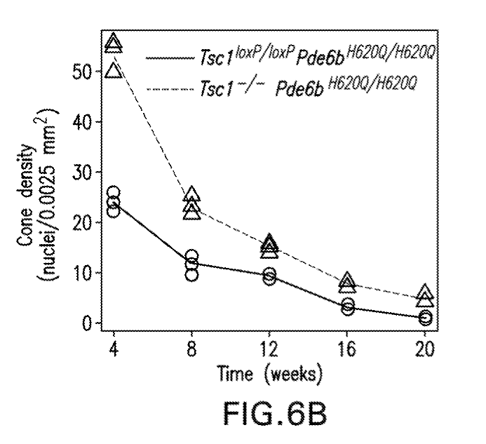

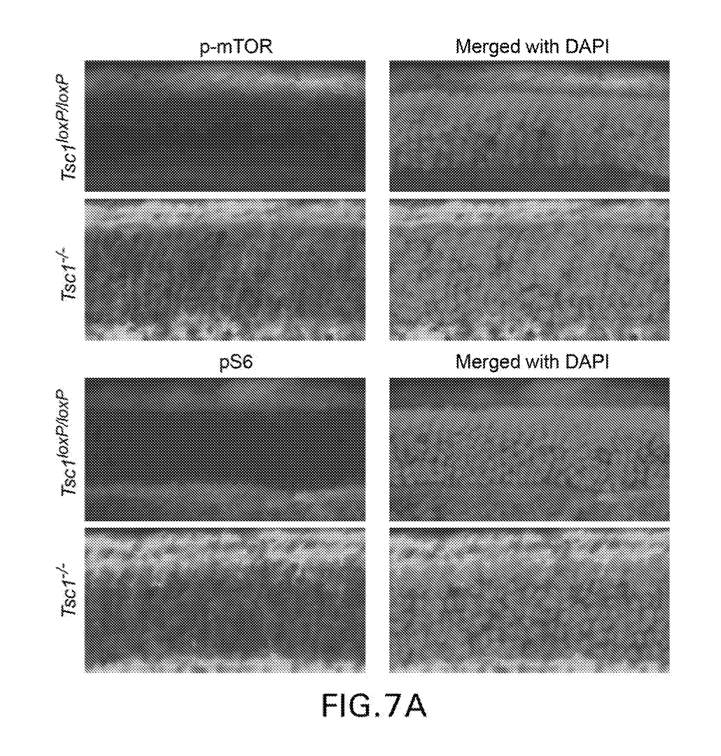

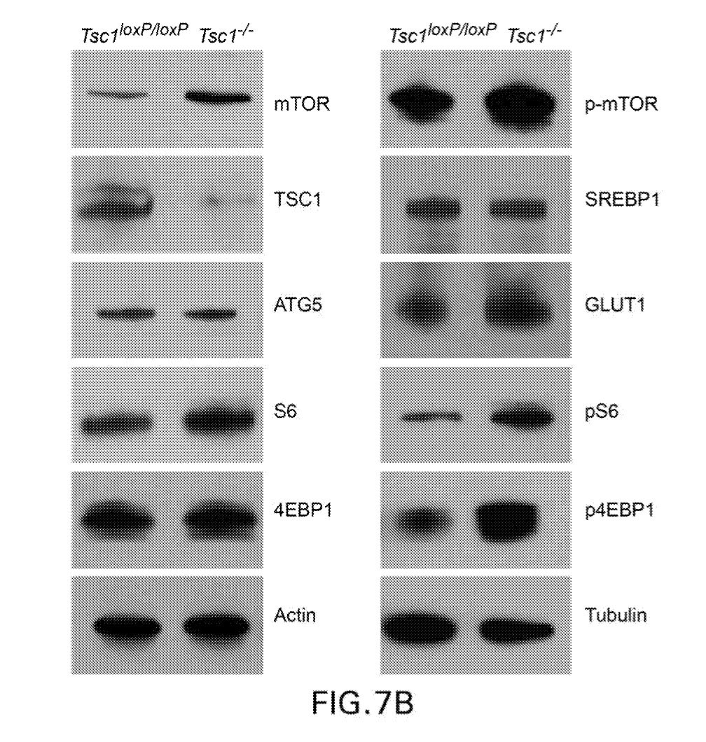

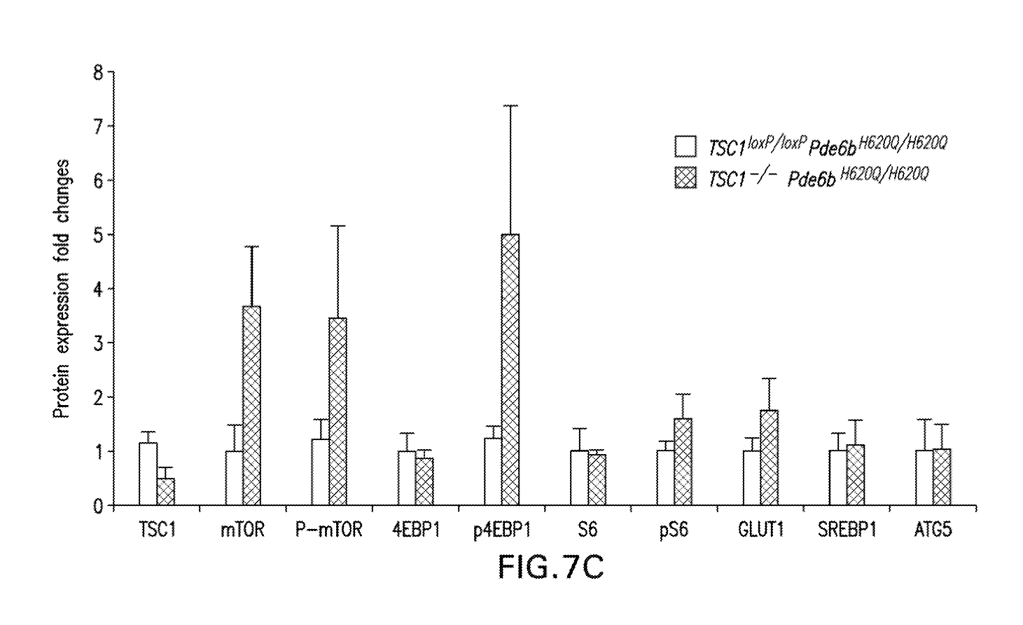

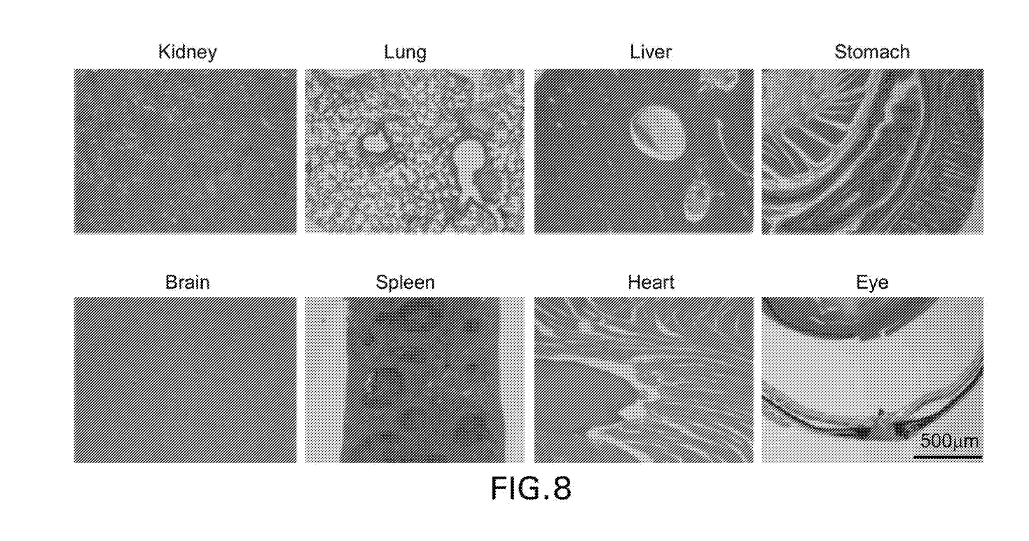

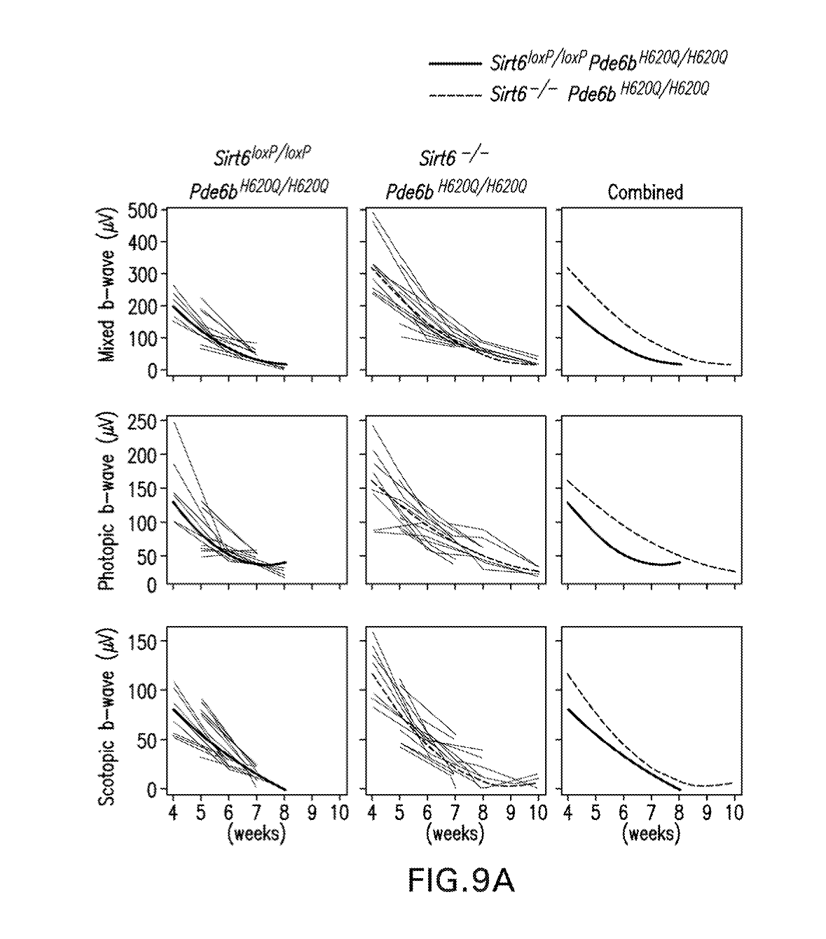

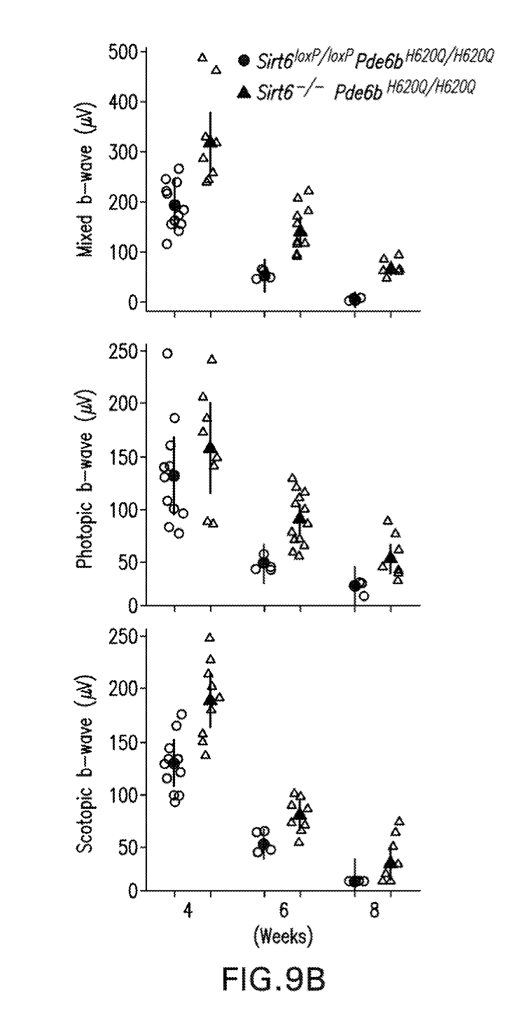

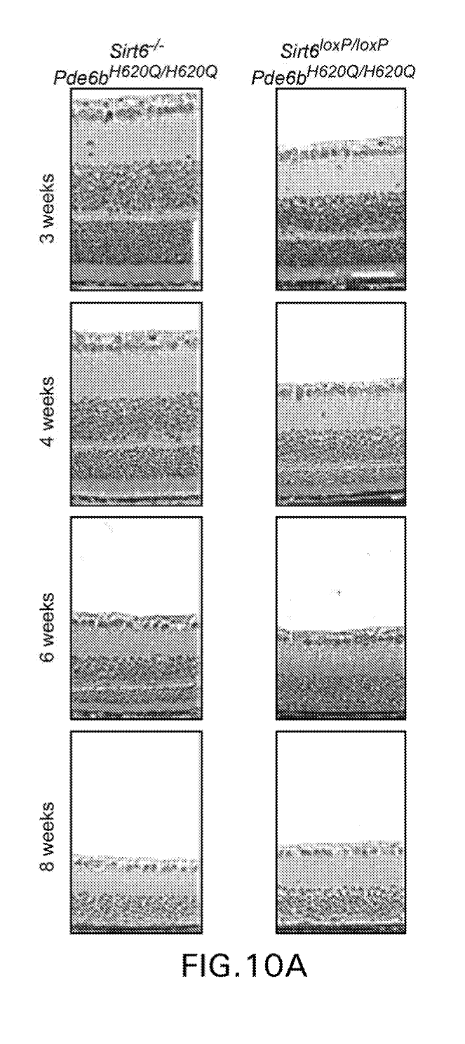

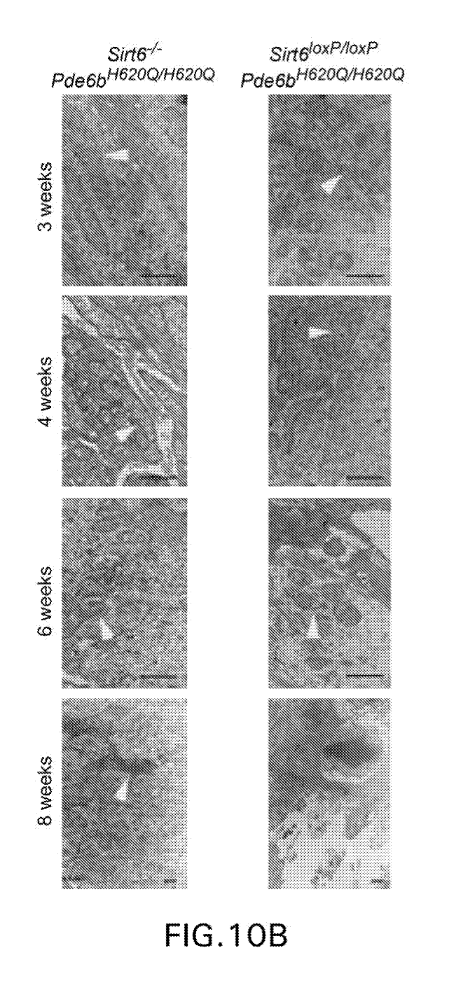

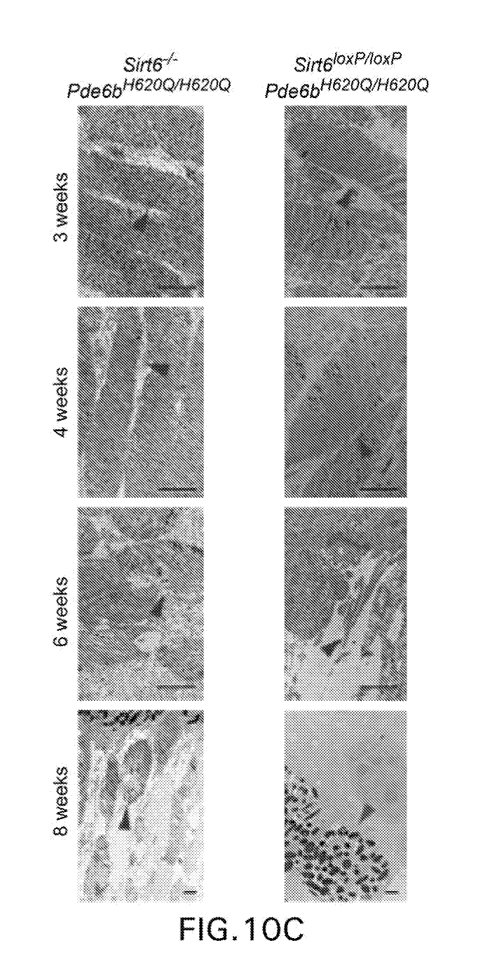

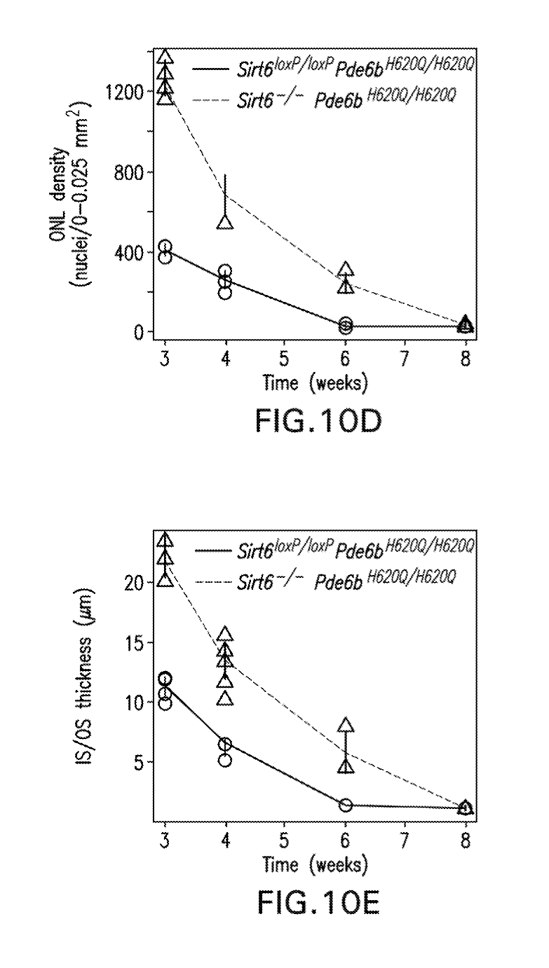

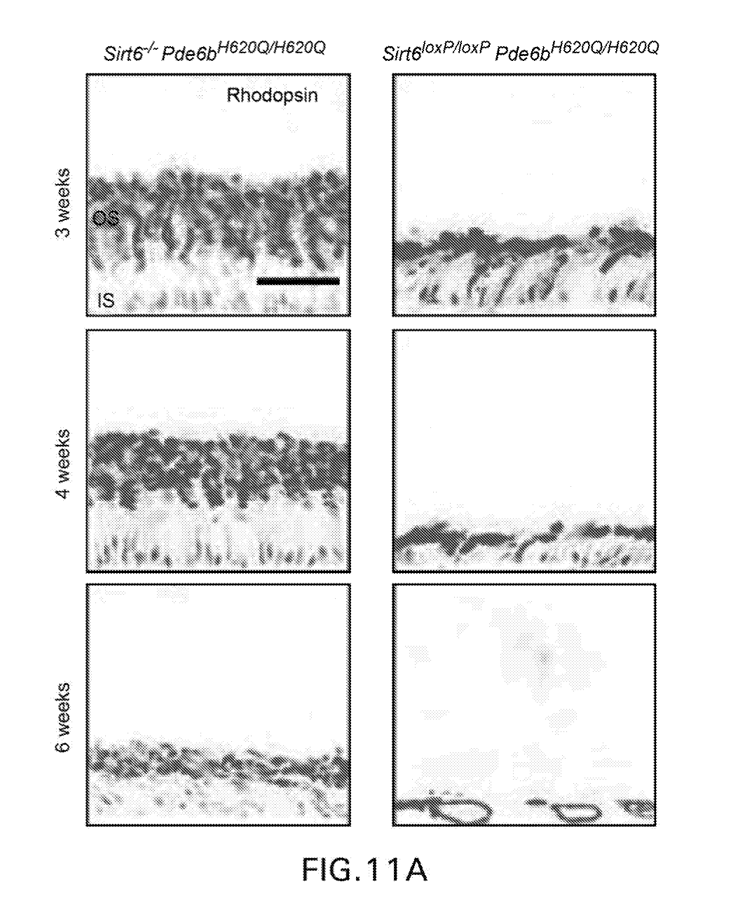

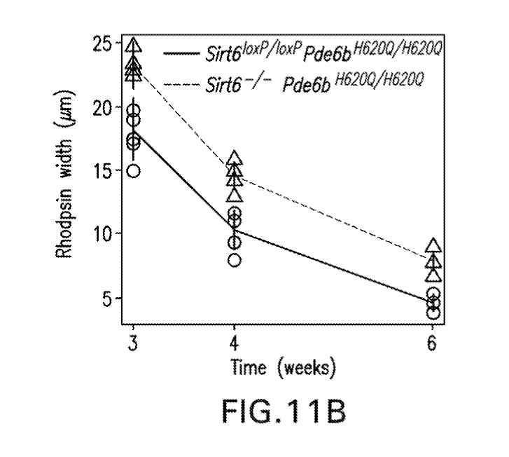

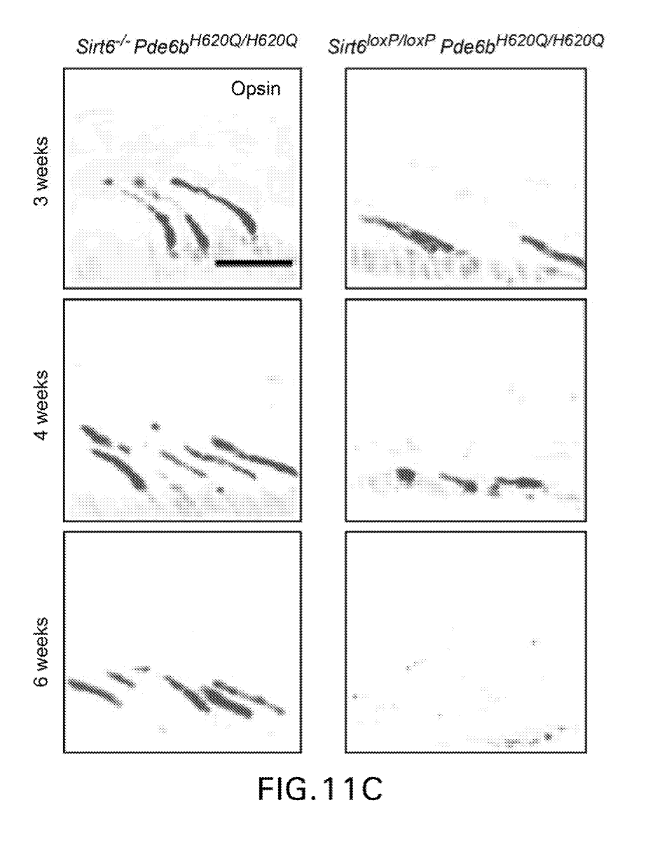

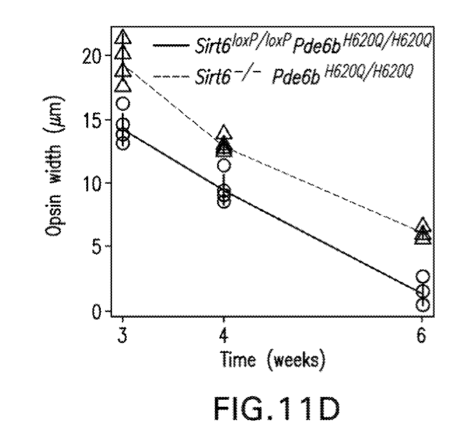

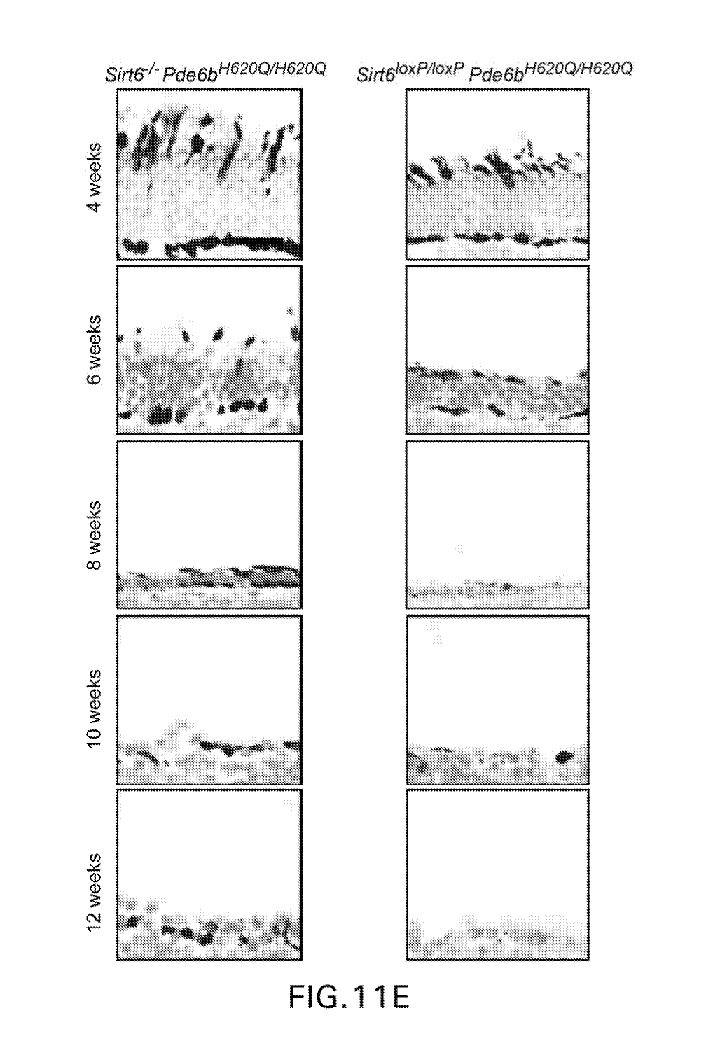

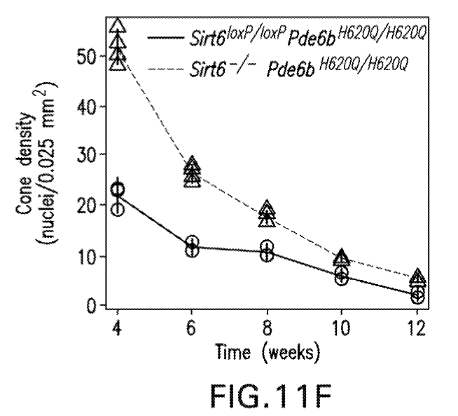

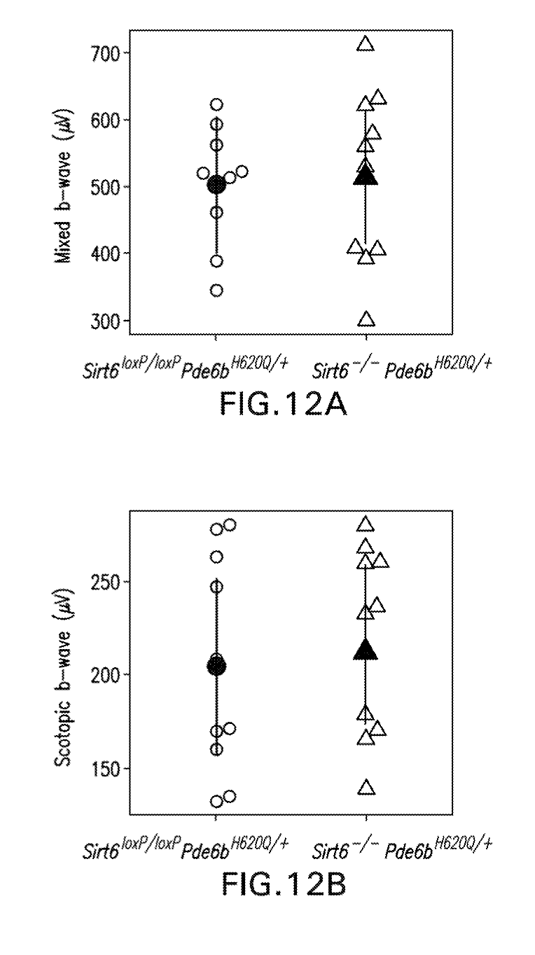

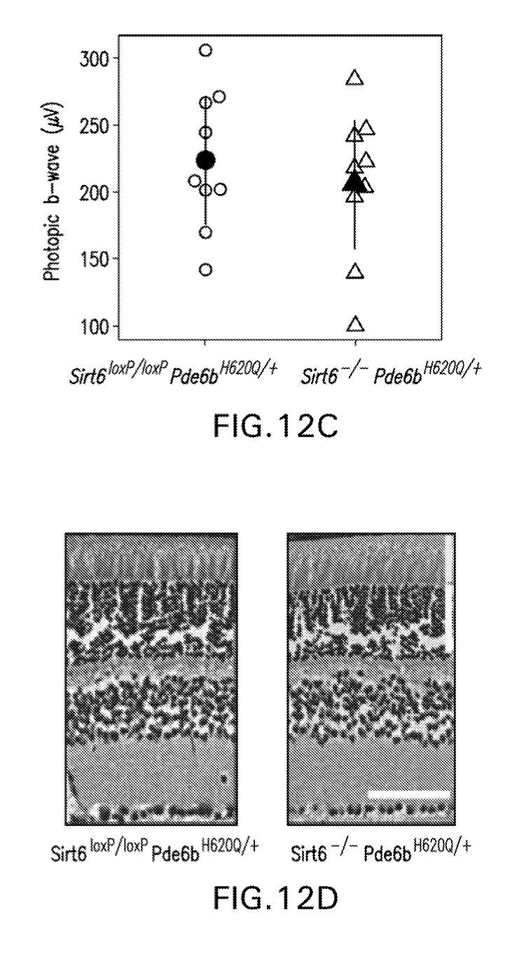

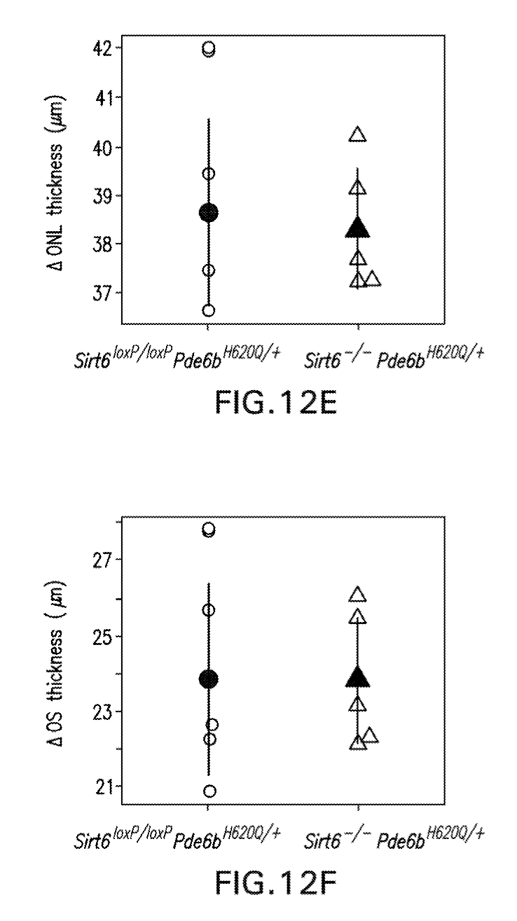

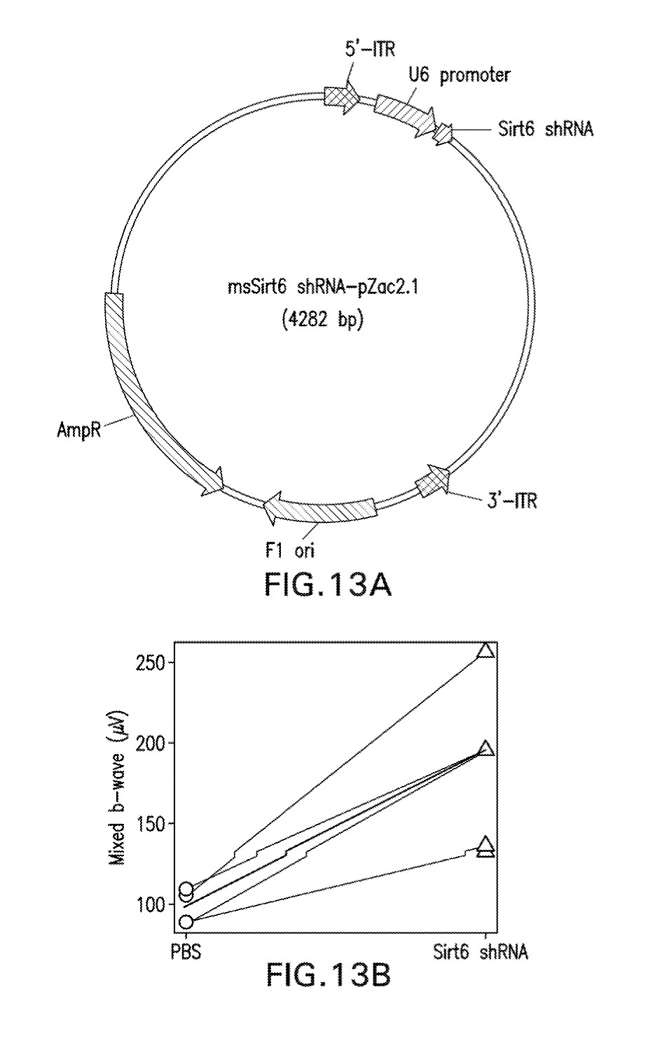

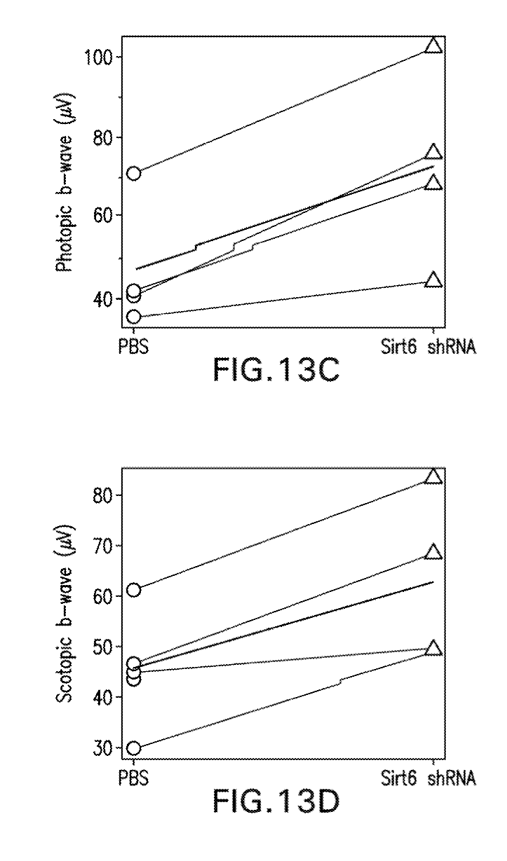

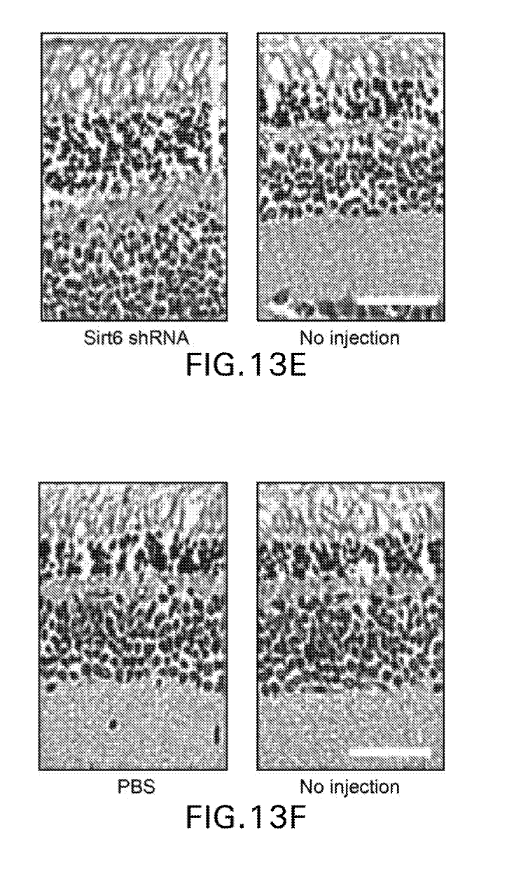

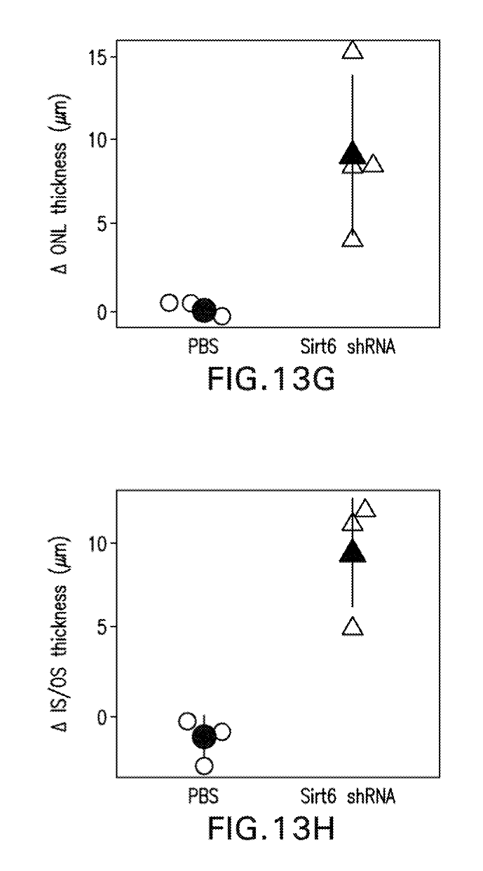

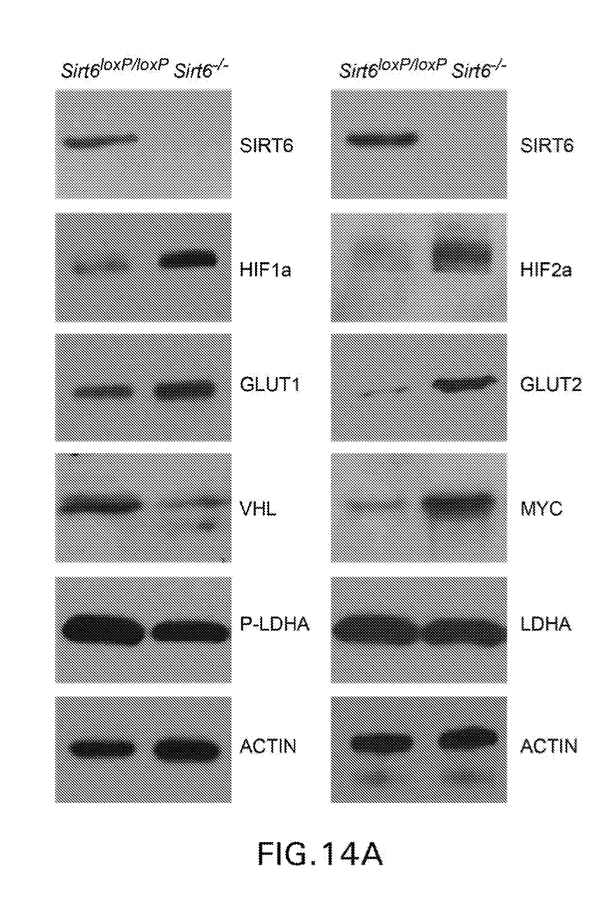

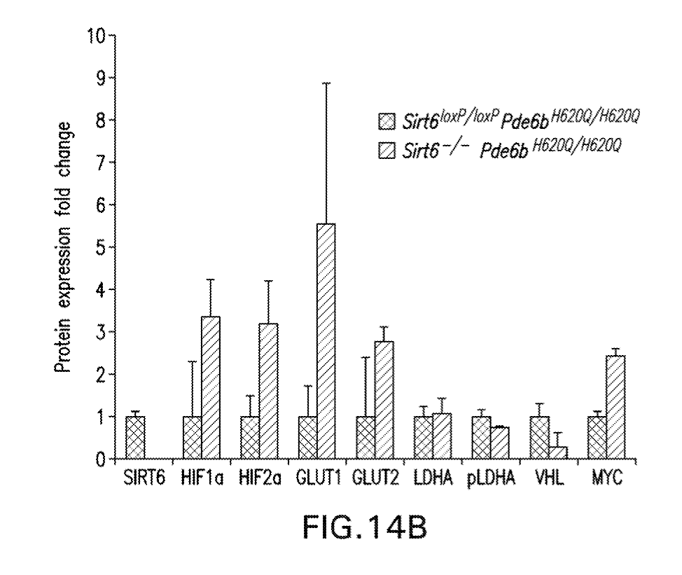

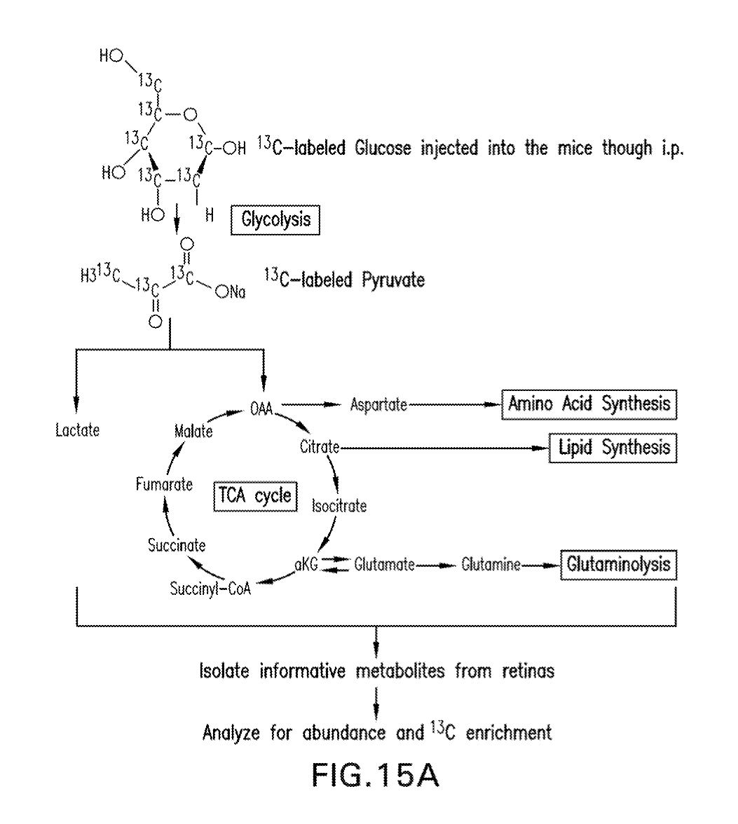

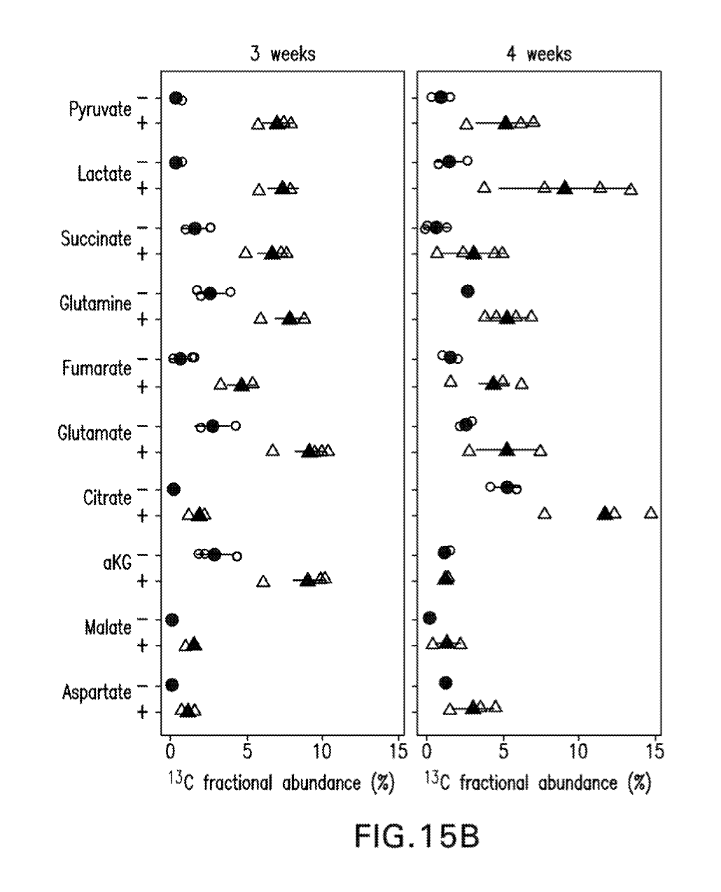

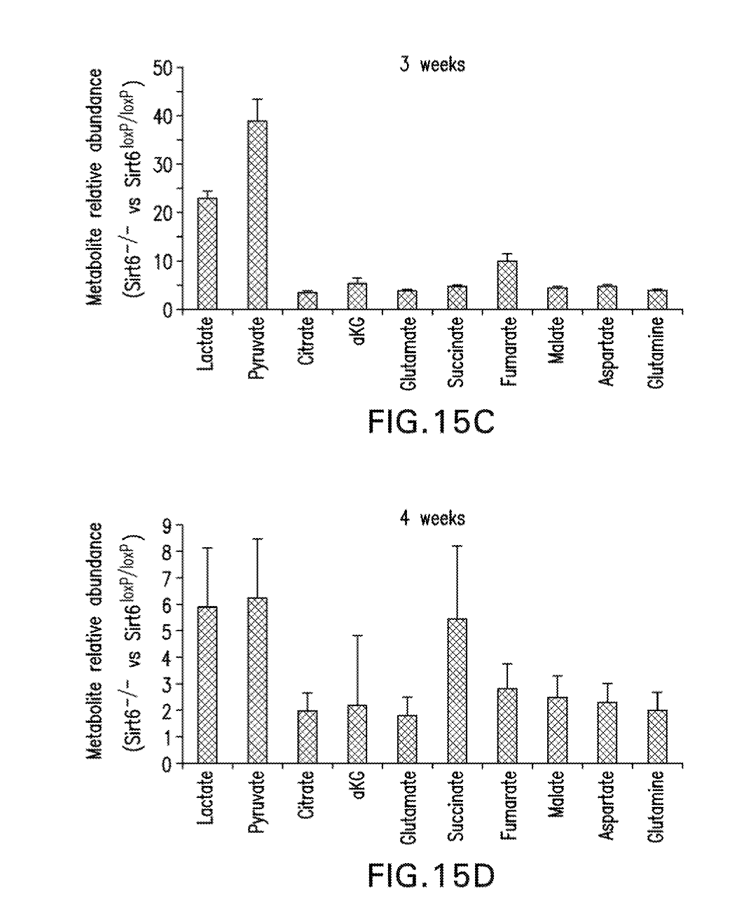

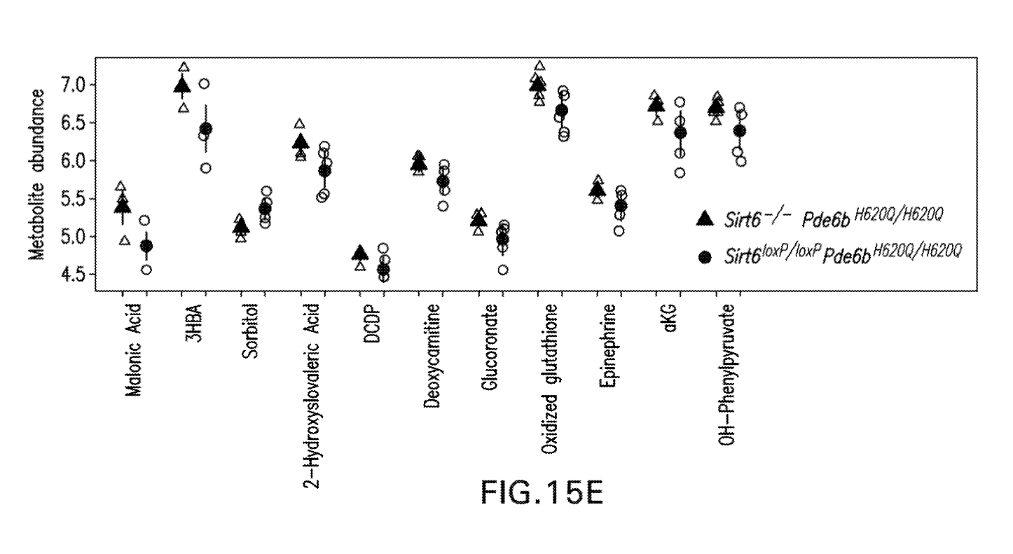

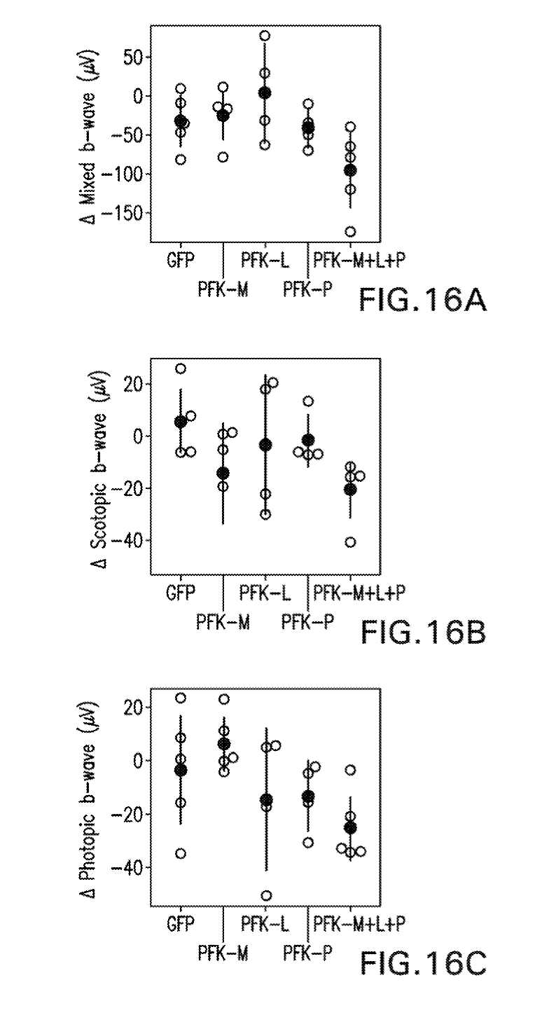

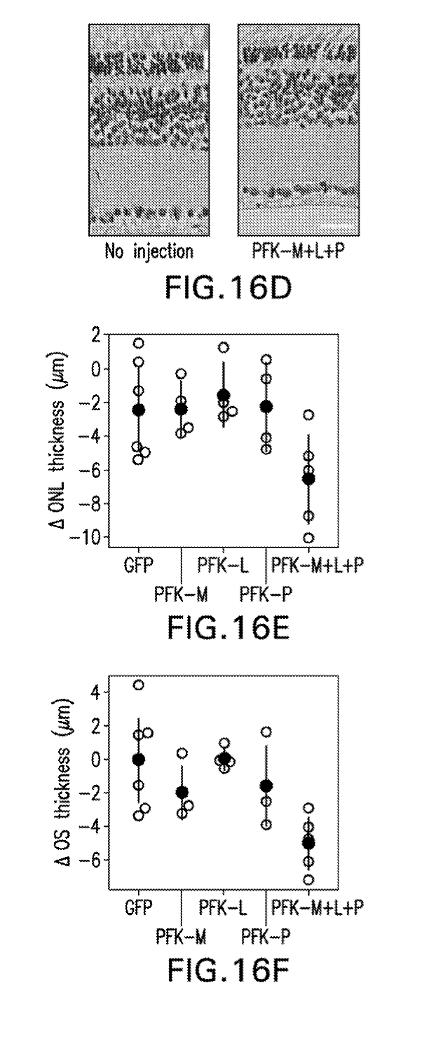

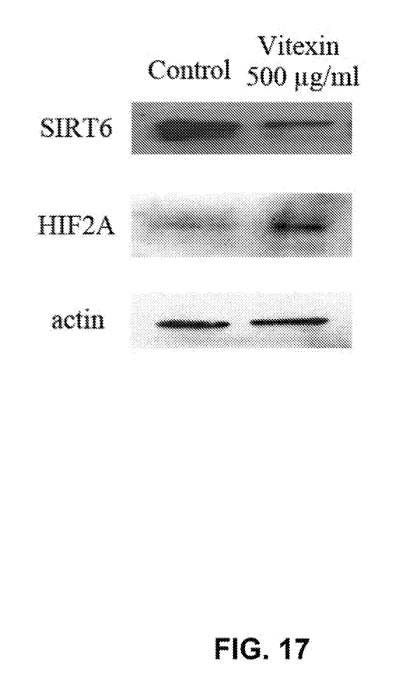

(Yellow bar indicates ONL, white bar indicates IS/OS, scale bar=μm.). FIG. 2B illustrates that ONL nuclei density a ONL layer width were greater in Tsc1^{-/-} [symbol] mice compared to Tsc1^{loxP/loxP} [symbol] mice at every time point. IS/OS width was quantified and observed to both groups, although the Tsc1^{-/-} [symbol] group had a greater mean thickness at each time point compared to the controls. Predicted trend lines for the ONL had steeper slopes for the control mice compared to the experimental group for both nuclei density and layer thickness. IS/OS trend lines were positively sloped and steeper for the experimental group compared to controls. (Grey dots represent individual mouse data points. Black lines connect the mean outcome at each time point; red lines are estimated mean trend lines from linear regression models, assuming linearity of the outcome over time. Dashed lines are used for controls, and solid lines are used for knockout mice,. Comparisons of Tsc1^{-/-} [symbol] versus Tsc1^{loxP/loxP} [symbol] controls at fixed points: ONL nuclei density: P17<0.001, P24 P<0.001; ONL width; P17 P<0.001; P24 P=0.001. IS/OS width: P17 P=0.02, P24 P=0.009, Comparison of the slopes between the two groups from the linear regression models: ONL density: P=0.02, ONL width P=0.003, IS/OS P=0.06, P10 n=4 for both groups. P17 and P24 n=5 for Tsc1^{-/-} [symbol] and n=4 for Tsc^{loxP/loxP} [symbol] ).

FIGS. 3A-B. Tsc1^{-/-} [symbol] have improved retinal survival compared to Tsc1^{loxP/loxP} [symbol] controls for at least 10 weeks. FIG. 3A: H&E-stained retinal sections were compared at 4, 5, 6, 8, and 10 weeks. Mean ONL and IS/OS thickness were greater at every time point in the Tsc1^{-/-} [symbol] group, compared to controls. Yellow bar indicates ONL, white bar indicates IS/OS, scale bar=20 μm.). FIG. 3B; This difference was quantified by counting ONL nuclei density and measuring the width of the ONL and IS/OS over time. Tsc1^{-/-} [symbol] mice had higher mean nuclei numbers and mean layer width in the ONL and IS/OS at all time points compared to controls, although the differences between the groups diminished over time. The predicted trend line for the ONL nuclei density and width was negative for both groups but steeper for the treated group. The IS/OS predicted trend line between the groups had comparable slopes. (Grey dots represent individual mouse data points. Black lines connect the mean outcome at each time point. Red lines are estimated mean trend lines from linear regression models, assuming linearity of the outcome over time. Dashed lines are used for controls, and solid lines are used for knockout mice. Comparisons of Tsc1^{-/-} [symbol] versus Tsc1^{loxP/loxP} [symbol] at fixed points: ONL, nuclei density: Week 4 P<0.001, Week 5 P=0.10, Week 6 P=0.03, Week 8 P=0.75, Week 10 P=0.33; ONL width: Week 4 P<0.001, Week 5 P=0.06, Week 6 P=0.002, Week 8 P=0.49, Week 10 P=0.27; IS/OS width: Week 4 P 0.002, Week 5P=0.02, Week 6 P<0.001, Week 8 P 0.65, Week 10 P 0.12, Comparison of the slopes between the two groups from the linear regression models: ONL density P=0.06, ONL as width P=0.05, IS/OS P=0.39. First n value represents Tsc1^{-/-} [symbol] , and second a represents Tsc1^{loxP/loxP} [symbol] ; Week 4 n=9 and n=6, Week 5 n=5 and n=4, Week 6 n=9 and n=4, Week 8 n=4 and n=6, Week 10 n=5 and n=6.).

FIGS. 4A-B. Retinal function was statistically improved in Tsc1^{-/-} [symbol] mice compared to controls. FIG. 4A: ERG scotopic, photopic, and mixed b-wave amplitudes were acquired over time, and mean b-wave responses were higher in Tsc1^{-/-} [symbol] mice compared with controls. (Grey lines are eyes from individual mice. From the linear mixed models, solid black lines are estimated mean trajectories for the controls, and dashed black lines are for the experimental group. Likelihood ratio tests: Scotopic P<0.001, Mixed P=0.002, Photopic P=0.01. Week 4 both groups n=6, Week 6 n=6 for Tsc1^{-/-} [symbol] n=5 for Tsc1^{loxP/loxP} [symbol] , Week 8 both groups N=5, Week 10 both n=4, Week 12 n=4 for Tsc1^{-/-} [symbol] , n=0 for Tsc1^{loxP/loxP} [symbol] ). FIG. 4B: Comparisons of knockout versus control mice at fixed points revealed that mean b-wave recordings were higher at every time point in the Tsc1^{-/-} [symbol] group compared to controls and were statistically significant at each time point. (Small grey dots represent data points from individual eyes; bold red dots represent means for the Tsc1^{loxP/loxP} [symbol] , while bold black dots represent means for Tsc1^{-/-} [symbol] , Scotopic: Week 4 P=0.02, Week 6 P=0.08, Week 8 P=0.04. Mixed: Week 4 P=0.003, Week 6 P=0.003, Week 8 P=0.01 Photopic: Week 4 P<0.001, Week 6 P=0.001, Week 8 P=0.02.).

FIGS. 5A-B., RHO immunoreactivity reveals rod preservation over 12 weeks in Tsc1^{-/-} [symbol] mice. FIG. 5A: Immunofluorescence staining of RHO was more intense in the IS/OS layer of Tsc1^{-/-} [symbol] mice compared to controls at every time point. (Scale bar=20 μm.). FIG. 5B: The width of RHO-expression in the IS/OS was measured at 300 microns from the optic nerve and quantified. A statistically greater mean width in the experimental group compared to controls was found at every time point. (Grey circles represent data from individual control mice; grey triangles represent individual experimental mice. The solid black line connects means for controls, and the dashed black line represents means for the experimental group. Week 4 P=0.008, Week 6 P<0.001, Week 8 P=0.005, Week 10 P=0.006, Week 12 P<0.001. Week 4 n=6 for both groups, Week 6n=4 for Tsc1^{-/-} [symbol] n=5for Tsc1^{loxP/loxP} [symbol] . Week 8 n=5 for Tsc1^{-/-} [symbol] , n=4 for Tsc1^{loxP/loxP} [symbol] , Weeks 10 and 12 n=4 for both groups.).

FIGS. 6A-B Cones arrestin immunoreactivity reveals suggest cone preservation of time in Tsc1^{-/-} [symbol] mice, FIG. 6A: Cones were stained with anti-cone arrestin antibody at multiple time points. Higher mean anti-cone arrestin immunoreactivity was observed in the Tsc1^{-/-} [symbol] mice, compared to controls at every time point. In both groups, there was a decline in staining over time. (Scale bar=20 μm), FIG. 6B: Photoreceptor nuclei numbers were quantified, and a statistically significant higher mean density was observed for the experimental group at every time point. (Grey circles represent data from individual mice from the control group; grey triangles represent individual mice from the experimental group. The solid black line represents means for controls, and the dashed black line represents means for the experimental group. For all time points, n=4 for both groups, and P<0.001, except Week 20, where P=0.002.

FIGS. 7A-C. Downstream targets of mTOR are upregulated in Tsc1^{-/-} [symbol] mice. FIG. 7A: Increased expression of phosphorylated mTOR and the downstream protein S6 was detected in immunofluorescence staining in the IS/OS layers as well as the ONL in the experimental mice. Merged DAPI images of nuclei revealed the location of the ONL. (Scale bar=20 μm.). FIG. 7B: Immunoblot revealed upregulated mTOR, p-mTOR, pS6, GLUT1, and p4EBP1, expression levels and downregulated TSC1 levels in the experimental versus control groups. ATG5 expression was not significantly affected by Tsc1 ablation. Actin and tubulin served as loading controls. FIG. 7C: Protein expression was quantified by analyzing the fold change between control and experimental groups. There was a statistically significant increase in mean protein expression in TSC1 in the experimental group compared to the controls. (TSC1 P<0.05, mTOR P<0.05, p-mTOR P=0.07, 4EBP1 P=0.49, p4EBP1 P<0.05, S6 P=0.78,)pS6 P<0.05, GLUT1 P<0.1, SREBP P=0.76. ATG5 P=0.95. n=4 for both groups.).

FIG. 8 are images showing no tumor formation up to 16 months following Tsc1 ablation in [symbol] mice. Tsc1^{-/-} [symbol] mice were observed for sixteen months. Major organs were subsequently harvested and subjected to H&E staining, which did not reveal tumor formations in any tested organs. (n=5).

FIGS. 9A-B. Sirt6 deficiency enhances electrophysiological function in both rods and cones. FIG. 9A: ERG data were obtained at weekly intervals under dark- and light-adapted conditions to scotopic, photopic, and mixed rod-cone b-wave amplitudes (μV). Linear mixed models were fit to estimate the trajectory of ERG outcomes over time, and differences were assessed by likelihood ratio tests. Gray and light red lines represent individual eyes; solid black and dashed red lines represent mean trajectories from the mixed models for Sirt6^{loxP/loxP} [symbol] and Sirt6^{-/-} [symbol] , respectively. Sirt6^{-/-} [symbol] mice had higher trajectories compared to control mice for each outcome: mixed (P<0.001), photopic (P=0.048), and scotopic (P=0.004). (For Sirt6^{loxP/loxP} [symbol] ; at 4 weeks, n=7; at 5 weeks, n=6; at 6 weeks, n=7; at 7 weeks, n=9; at 8 weeks, n=5; at 5 weeks, n=6; at 6 weeks, n=7; at 7 weeks, n=9; at 8 weeks, n=5;at 10 weeks, n=3). FIG. 9B: comparison of ERG data under scotopic, photopic, and mixed conditions at 4, 6, and 8 weeks is provided. Gray and light red dots represent data from individual mice, whereas black dots and red triangles are means for Sirt6^{loxP/loxP} [symbol] and Sirt6^{-/-} [symbol] , respectively. There were statistically significant differences between the experimental and control mice at all time points and all light adaptation conditions except for photopic conditions at 4 weeks and scotopic at 8 weeks. A linear mixed model was fit to compare groups. Error bars reflect standard deviations. (At 4 weeks: scotopic b-wave P=0.004, mixed b-wave P=0.01, photopic b-wave P=0.3; at 6 weeks: scotopic b-wave P=0.007, mixed b-wave P<0.001, photopic b-wave P=0.03; at 8 weeks: scotopic b-wave P=0.2, mixed b-wave P<0.001, photopic b-wave P=0.048).

FIGS. 10A-E are images and graphs showing that Sirt6 deficiency improves both rod and cone survival and robustness. FIG. 10A: H&E staining of paraffin sections of retinae showed thicker ONL, and IS/OS layers and a greater concentration of photoreceptor nuclei in Sirt6^{-/-} [symbol] compared with Sirt6^{loxP/loxP} [symbol] mice at 3, 4, and 6 weeks. (Scale bar=50 μm. White vertical bar represents the ONL, while yellow vertical bar represents IS/OS.). FIG. 10B: TEM of the photoreceptors' mitochondria (yellow arrowheads) showed that they are healthier, with more cristae and longer cells bodies in the Sirt6^{-/-}mice compared with Sirt6^{loxP/loxP} [symbol] mice. An observable difference between the mitochondria of each group was detected until 8 weeks, at which point both group's mitochondria appeared diseased. (Scale bar=1 μm). FIG. 10C: TEM of OS (red arrowheads) of photoreceptors showed that their discs are more plentiful and the length of each OS is much greater in the Sirt6^{-/-} [symbol] compared with Sirt6^{loxP/loxP} [symbol] mice. There is a decline over time in both groups, although by 8 weeks, the knockout group still retained delectable OS while the control group appeared completely deficient of them. (Green arrowhead points to the boundary between the RPE cells and the biopolar cells' nuclei. Scale bar=1 μm). FIG. 10D-E: Statistical analysis of the ONL density (nuclei/0.0025 mm²) (FIG. 10D) and IS/OS thickness (μm) (FIG. 10E) corroborated histological findings, confirming that Sirt6-deficient mice had significantly higher density and thickness of the ONL and IS/OS layers, respectively. Two-tailed t tests were used to analyze the data. (ONL cell density 3, 4, and 6 weeks P<0.001; IS/OS thickness at 3and 4 weeks, P<0.001;at 6 weeks P=0.002;at 8 weeks, P=0.6 for both ONL density and IS/OS thickness. For Sirt6^{loxP/loxP} [symbol] : n=4 at 3 and 6 weeks, n=5 at 4 weeks, n=3 at 8 weeks; for Sirt6^{-/-} [symbol] : n=5 at 3 and 4 weeks; n=4 at 6 and 8 weeks).

FIGS. 11A-F are images, blots and graphs showing that Sirt6 deficiency promotes photoreceptor survival and preserves cellular OS. FIGS. 11A-B: Rhodopsin antibody staining of retinal OS layers was used to compare morphological differences between control and treated mice. The expression of rhodopsin was greater in the Sirt6^{-/-} [symbol] mice compared with controls at every time point. The width of rhodoposin was subsequently quantified in ImageJ (FIG. 11B). Gray dots represent data from individual Sirt6^{loxP/loxP} [symbol] mice, and a black trend light was projected to connect the means at each time point. Similarly, light red triangles represent individual data points from Sirt6^{-/-} [symbol] mice, and the red dashed line represents a projected trendline to connect the means of the Sirt6^{-/-} [symbol] mice at each time point. Two-tailed t tests were used to analyze the data. (3 weeks: P=0.012. 4 weeks: P=0.002. 6 weeks: P<0.001. n=5 at all time points. Scale bar=20 μm) FIGS. 11C-D: Short-wavelength cone opsin antibody (blue) staining of retinal OS layers was used to compare morphological differences between control and treated mice. The OS of cones are elongated in treated mice, with a greater density of cell nuclei compared with controls at every time point. Quantification by ImageJ confirmed observations (FIG. 11D). Graphical representations and statistical analyses are identical as indicated in FIGS. 11A-B. (3 weeks: P<0.001. 4 weeks; P=0.001, 6 weeks: P<0.001. n=5 at all time points. Scale bar=20 μm). FIGS. 11E-F: DAPI (blue) and anti-cone arrestin (green) staining allowed visualization and quantification of retina nuclei and cone cell markers, respectively. Results were merged into composite images. There is a greater density of cones at all time points in the experimental mice compared to controls. (Scale bar=20 μm). Cone density (nuclei/0.0025 mm²) was greater in the Sirt6 knockout mice at every time point compared with controls (FIG. 11F) (P<0.001 at 4, 6, 8, and 10 weeks, P=0.0016 at 12 weeks). Graphical representations and statistical analyses are identical as indicated in FIGS. 11A-B. (For Sirt6^{loxP/loxP} [symbol] n=3 at all time point, for Sirt6^{-/-} [symbol] ^{: n=}4 at all time points).

FIGS. 12A-F are traces and images showing that Sirt6 deficiency in wild type background produces no phenotypic changes in functionality or morphology of photoreceptors. FIG 12A-C: ERG mixed, scotopic, and photopic b-wave values showed no statistically significant difference between Sirt6^{-/-} [symbol] and Sir6^{loxP/loxP} [symbol] mice. Recordings were taken 3 months post tamoxifen injection. Gray dots represent values from individual Sirt6^{loxP/loxP} [symbol] mice, while the black dot represents the mean Similarly, light red triangles represent values from individual Sir6^{-/-} [symbol] mice, and the red dot, the mean. (Error bars show standard deviation. n=5 for both groups.). FIG. 12D-F: H&E-stained retinal sections were collected from both groups at 4 months post tamoxifen injection. No observable differences in ONL or OS thickness were observed. Layer widths were quantified, and results confirmed observed histological findings. (Graphical representations are identical as those described in A-C. n=5 for both groups. White vertical bar represents ONL; yellow bar represents IS/OS. Scalebar=50 μm.).

FIGS. 13A-H. Gene therapy improves function and survival of photoreceptors in a preclinical RP model. FIG. 13A: Schematic representation of the AAV2/8(Y733F)-Sirt6_ShRNA pZac:2.1 vector with the U6 promoter driving expression of the Sirt6_shRNA. Arrows indicate the direction of transcription. 5′- and 3′-ITR=inverted terminal repeats of AAV; AmpR ampicillin resistance gene; F1 ori=origin of replication. FIGS. 13B-D: Mixed, photopic, and scotopic ERG b-wave recordings (μV) showed a statistically significant improvement in the AAV2/8(Y733F) Sirt6_shRNA-injected eye compared with the PBS-injected eye at four weeks post injection. Gray dots represent PBS-injected eyes, while red triangles represent Sirt6_shRNA vector-injected eyes. Gray lines connect eyes from the same mouse: black lines indicate differences in means between PBS-Injected vs. vector-injected eyes. Paired/tests were used to compare vector-injected and PBS-injected eyes at each time point. (Mixed: P=0.02. Photopic: P=0.02. Scotopic; P=0.02. n=4 for all groups). FIG. 13E-H: H & E stained retinal sections were taken from a 4-week-old [symbol] mouse that was injected dorsally with Sirt6_shRNA in the right eye (FIG. 13E, left). The ventral side of the right eye was not injected but was used as a control (E, right). In the left eye, PBS was injected dorsally (FIG. 13F, left) while the ventral side served as a control (FIG. 13F, right). The dorsal side of the AAV-injected right eye shows retinal rescue (FIG. 13G), while control (PBS-injected) and untreated sites on the ventral side of each eye show continued degeneration (FIG. 13H). Gray dots represent values from individual PBS-injected retinae, while the black dot represents the mean thickness. Similarly, light red triangles represent values from individual Sirt6 shRNA injected retinae, and the red dot, the mean. (Error bars represent standard deviation, ONL:P=0.03. IS/OS: P=0.004, n=4 for all groups. White vertical bar represents ONL; yellow bar represents IS/OS. Scale bar 50 μm).

FIGS. 14A-B. Sirt6 deficiency op-regulates levels of glycolytic metabolism intermediates. FIG. 14A: Immunoblotting for regulators of glycolytic metabolism in the retinae of treated and untreated mice at P21 revealed increased levels of hypoxia-inducible factors 1A (HIF1A) and 2A (HIF2A); glucose transporters 1 (GLUT1) and 2 (GLUT2); and MYC in Sirt6^{-/-} [symbol] mice. There was a corresponding decrease in von Hippel-Lindau (VHL)) protein and p-lactate dehydrogenase A (p-LDHA). No difference was detected for LDHA between the groups. β-actin (ACTIN) was used as a loading control. FIG. 14B: Protein expression was quantified to assess between Sirt6^{-/-} [symbol] and Sirt6^{loxP/loxP} [symbol] , and a paired t-test was used to determine statistical significance. HIF1a, HIF2a, GLUT1, GLUT2,and MYC were found to be increased in the Sirt6^{-/-} [symbol] mice compared to controls. SIRT6, VHL, and p-LDHA were found to be decreased in the Sirt6^{-/-} [symbol] group. No difference between groups was found for LDHA, (SIRT6 P<0.001, HIF1a P=0.02, HIF2a P=0.01, GLUT1 P=0.04, GLUT2 P=0.048,LDHA P=0.68, p-LDHA P=0.04, VHL P=0.02, MYC P<0.001. n=4 for both groups.)

FIGS. 15A-E. Lack of the nun of Sirt6 increases the flow of carbons from glucose to downstream metabolites. FIG. 15A: A schematic representation of glycosis and the TCA cycle, highlighting key metabolic intermediates that were compared in control and experimental groups in (FIG. 15B). ¹³C-labeled glucose injected intraperitoneally (i.p.) is converted into pyruvate, which is reduced into lactate or oxidized in the mitochondria through the TCA cycle. Red font indicates incorporation of the ¹³C isotope into the downstream metabolites evaluated by GC-MS. FIG. 15B: Sirt6^{-/-} [symbol] mice have consistently higher levels¹³C-labeled metabolites as compared with the Sirt6^{loxP/loxP} [symbol] control mice. The graphs show the ratio of ¹³C enrichment of each metabolite 45 minutes after injection of ¹³C-labeled glucose at 3 and 4weeks. At 3 weeks, there was a statistically significant difference in all metabolite levels in the control versus experimental mice. At 4 weeks, the statistical significance diminished but remained present for some of the metabolites: pyruvate, citrate, glutamine, lactate. Gray dots represent values from individual Sirt6^{loxP/loxP} [symbol] mice, while blade dots represent the mean. Similarly, light red triangles represent values from individual Sirt6^{-/-} [symbol] mice, and the red dot, the mean. (−) represents Sirt6^{-/-} [symbol] mice, while (+) represents Sir6^{-/-} [symbol] mice. Two-tailed t tests used to analyze the data. Error bars represent standard deviation. (P<0.001 for all metabolism at 3 weeks. P<0.05 or pyruvate, citrate, glutamine, and lactate at 4 weeks. P not significant for other metabolites. n=4 for Sirt6^{-/-} [symbol] and Sirt6^{loxP/loxP} [symbol] mice at 3 and 4 weeks.). FIGS. 15C-D: Statistical analysis to evaluate the relative metabolic abundance fold change of glycolytic and TCA cycle intermediates at three weeks of age in the Sirt6-deficient versus control mice showed that all metabolites were upregulated, although the glycolytic metabolites, namely lactate and pyruvate were significantly more upregulated compared with the TCA cycle intermediates. At four weeks, a similar trend was observed, but the significance was diminished, although the lactate and pyruvate still maintained a significantly greater abundance compared with the TCA cycle intermediates. (At three weeks, all metabolites: P<0.001, n=4 for both groups. At four weeks, lactate: P=0.009; pyruvate: P=0.005; glutamine: P=0.02; malate: P=0.03; fumarate: P=0.04; aspartate: P=0.03. All others, P>0.05. n=4 for both groups.). FIG. 15E: LC-MS (without ¹³C labeling) revealed eleven significantly increased downstream metabolites of glycolysis and the TCA cycle in the Sirt(6^{-/-} [symbol] retinae compared with the Sirt6^{loxP/loxP} [symbol] retinae at three weeks of age. Two-tailed t tests were used to analyze the data. (P<0.05 for all metabolites, n=6 per group.

FIGS. 16A-F. PFK shRNA viral knockdown exacerbates retinal degeneration in [symbol] mice. FIGS. 16A-C: ERG mixed, scotopic, and photopic b-wave recordings were obtained from [symbol] mice injected with a lentivirus expressing GFP, PFK−M, PFK−L, PFK−P, or PFK−P+M+L shRNA. A statistically significant decrease in b-wave values under scotopic conditions was detected when PFK−P+M+L shRNA was introduced in the mice, and a notable but non-significant difference was detected under mixed and photopic conditions. Gray dots represent values from individual mice, While the black dot represents the mean. Error bars show standard deviation. (Pairwise comparisons against GFP were conducted for each viral injection: Scotopic. PFK−M: P=0.12. P=0.57. PFK−L: P=0.38. PFK−P+M+L; 0:01. Mixed, PFK−M: P=0.74 PFK−L: P=0.37. PFK−P: P=0.66, P=0.06, Photopic. PFK−M: P=0.42. PFK−L: P=0.53. PFK−P: P=0.44. PFK−P+M+L: P=0.11. n=5 for all groups except PFK−L, where n=4.). FIG. 16D-F: Mice were injected dorsally with the lentivirus while the ventral side was left untreated. An observable difference was detected between the injected and non-injected sides in mice exposed to the lentivirus expressing PFK−P+M+L shRNA. The difference in the ONL and OS thickness was quantified and found to be statistically significant for the PEK−P+M+L injection group compared to GFP controls. Graphical representations were rendered as described above and showed that only the combination of PFK−P+M+L led to a statistically significant decrease in each layer. (Scale bar=20 μm. Pairwise comparisons against GFP were conducted for each viral infection: ONL. PFK−M: P=m. P=0.98. PFK−L; P=0.58. PFK−P: P=0.92. PFK−P+M+L: P=0.04. OS. PFK−M: P=0.24 PFK−L; P=PFK−P: P=0.41, PFK−P−M+L: P=0.01. r=5 for all groups except PFK−L, Where n=4.).

FIG. 17 is a immunoblot show that Vitexin (from English hawthorn berries) suppressed SIRT6 immunoreactivity and upgraded HIF2 glycolysis enhancer endothelial PAS domain protein 1 (HIF2A, hypoxia inducible transcription factor 2alpha.). Anti-beta- actin immunoreactivity was used as loading controls.

### DETAILED DESCRIPTION

Retinitis pigmentosa (RP) is an incurable neurodegenerative condition featuring photoreceptor death that leads to blindness. Currently, there is no approved therapeutic for photoreceptor degenerative conditions like RP and atrophic age-related macular degeneration (AMD). Although there are promising results in human gene therapy, RP is a genetically diverse disorder, such that gene-specific therapies would be applicable to only a small fraction of patients with RP. In RP, mutations in genes encoding phototransduction enzymes such as rhodopsin and phosphodiesterase 6 (PDE6) impair photoexcitation, creating imbalance between anabolic and catabolic processes that leads to shortening of the outer segments (OS) of photoreceptors and eventually triggering cell death (2,9-12). The OS is shed and regenerated daily, but in diseased photoreceptors, there are aberrations in the renewal cycle that lead to significantly shorter OS and subsequent dysgenesis (2,9). Augmenting anabolism could theoretically fuel protein and lipid synthesis, thus encouraging OS regenesis. This strategy could potentially serve as a treatment for RP by reprogramming rods towards anabolism, preserving their ability to maintain the OS, and increasing their chance of survival.

The mechanistic target of rapamycin (mTOR) pathway has been identified as a key regulator of anabolism, including such pathways as cellular metabolism and growth based on cues such as stress, hypoxia, growth factors and glucose concentration (13-15). Binding of growth factors like insulin activates the mTOR complex (mTORC), promoting anabolic processes such as ribosome biogenesis, protein synthesis and transcription (16,17), Simultaneously, catabolic processes like autophagy and apoptosis are suppressed (18,19). A similar effect is achieved in the presence of high levels of amino acids and nutrients. Previously, we collected preliminary data on the effects of mTOR upregulation by shRNA-mediated silencing of the tuberous sclerosis complex 2 (TSC 2) in a mouse model of RP and found increased photoreceptor numbers and preservation of function (20). Similar results were obtained by Punzo et al. when the mTOR pathway was upregulated in cones in four different mouse models of RP (21).

As described herein we determine the effects of a specific ablation of tuberous sclerosis complex 1 (Tsc1) in rods, which has not been explored before, using a tamoxifen-inducible, rod-specific Tsc1 knockout P [symbol] RP model. The [symbol] mutation prevents normal activation of the phototransduction cascade and features rod OS dysgenesis (20, 22). While heterozygous mice are phenotypically normal, homozygotes experience relatively rapid photoreceptor degeneration (20, 22). The present data support the hypothesis that upregulation of mTOR in rods by Tsc1 knockout can facilitate both rod and cone morphological and functional preservation and enhance survival of photoreceptor cells. Similarly, it is expected that these effects will also extend to neuronal survival and preservation in glaucoma as well as in neurodegenerative conditions including Alzheimer's disease, Parkinson's disease, Huntington's disease, amyotrophic lateral schlerosis (ALS), Lewy Body dementia, and any condition that will benefit in increased glycolysis leading to improved cell viability and longevity.

In addition to inhibition of TSC1, a second strategy we have explored involves knockdown of Sirtuin-6 (SIRT6). The historic deacetylase SIRT6 is a transcriptional repressor of glycolytic ewymes that has been extensively studied in the context of metabolism and cancer biology (39). Normally, SIRT6 directs glucose metabolism to proceed through an aerobic fashion by maintaining histone H3 (H3K9) in a deacetylated form and by repressing hypoxia inducible factor 1a (HIF1a), a transcription factor (40). When nutrients are scarce or when SIRT6 is systemically suppressed experimentally, glucose is preferentially processed through glycolytic pathways. We hypothesized that knockdown of SIRT6 in rod photoreceptors could theoretically remodel cellular metabolism to favor glycolysis over respiration (4143). This should increase the concentrations of intermediates in glycolysis and the pentose phosphate pathway, thereby enhancing the production of fuels for OS lipid synthesis and promoting survival. Previous studies in a conventional systemic knockout, Sirt6^{tm1Fwa}, were challenging to interpret because of negative effects on synaptic transmission (44). We therefore altered our approach to limit ablation of Sirt6 to rod photoreceptors with an inducible gene disruption strategy. Using this model, we provided evidence that up-regulation of glycolytic flux through Sirt6 knockout can preserve both rod as well as cone photoreceptors in a preclinical, Ped6-associated RP model.

In certain embodiments, a non-gene-specific strategy is utilized that entails reprogramming photoreceptors towards anabolism by upregulating the mTOR pathway. We conditionally ablated Tsc1, an mTOR inhibitor, in the rods of the [symbol] preclinical RP mouse model and observed, functionally and morphologically, an improvement in the survival of rods and cones at early and late disease stages. Similar findings were achieved with conditional ablation of Sirt6. These results elucidate the ability of reprogramming the metabolome to slow photoreceptor degeneration. This strategy may also be applicable to a wider range of neurodegenerative diseases, as enhancement of nutrient uptake is not gene-specific and is, implicated in multiple pathologies, Enhancing anabolism promoted neuronal survival and function and could potentially benefit a number of photoreceptor and other degenerative conditions.

It is noted that as used herein Tsc1 and Sirt6 can refer to the gene or the protein encoded for by the gene, as appropriate in the specific context utilized. Additionally, in certain contexts, the reference will be to the mouse gene or protein, and in others the human gene or protein as appropriate in the specific context.

While not wishing to be bound by theory, aspects of the present invention relate to methods for increasing anabolism and decreasing catabolism in desired cells, in particular, in desired neuronal cells. Embodiments of the present invention relate to increasing glycolysis in neuronal cells, leading to improved neuronal cell survival. Additional embodiments of the present invention relate to methods of increasing photoreceptor cell survival in desired patient populations, including in patients with retinal degenerative diseases such as RP, AMD, and glaucoma. Indeed, we hypothesized that enhancing anabolic processes can confer beneficial effects on cells undergoing neurodegeneration, and we have validated this theory in two different gene targets, namely Tsc1 and Sirt6. However, these genes and their transcribed proteins are among many others that are involved in a complex pathway which regulates cellular metabolism, any of which may also be potential targets for treating neurodegenerative conditions. Thus, the inhibition or upregulation of metabolic effectors, TSC1 and SIRT6 being two such examples. may also prove fruitful in the treatment of such conditions. This strategy may also be used in combination with gene therapies and neurotrophic factor administration for heightened treatment efficacy.

In accordance with the present invention, there may be numerous tools and techniques within the skill of the art, such as those commonly used in molecular immunology, cellular immunology, pharmacology, and microbiology. See, e,g., Sambrook et at. (2001) Molecular Cloning: A Laboratory Manual. 3rd ed. Cold Spring. Harbor Laboratory Press; Cold Spring Harbor, N.Y. Ausubel et al, eds, (2005) Current Protocols in Molecular Biology, John Wiley and Sons, Inc,: Hoboken, N.J.: Bonifacino et al, eds. (2005) Current Protocols in Cell Biology. John Wiley and Sons, Inc.: Hoboken, N.J.; Coligan et al. eds. (2005) Current

Protocols in Immunology, John Wiley and Sons, Inc,: Hoboken, N.J.; Coico et al, eds. (2005) Current Protocols in Microbiology, John Wiley and Sons, Inc.: Hoboken, N.J. Coligan et al. eds. (2005) Current Protocols in Protein Science, John Wiley and Sons, Inc.: Hoboken, N.J.; and Enna et al. eds. (2005) Current Protocols in Pharmacology, John Wiley and Sons, Inc.: Hoboken, N.J.

By “TSC1,” “Tsc1,” “Tsc1” is meant to inclue the DNA, RNA, mRNA, cDNA, recombinant DNA or RNA, or the protein arising from the tuberous sclerosis complex 1 gene. The human nucleotide sequence can be found at Gene ID: ^{7248.} The mouse nucleotide sequence can be found at Gene ID: 64930.

By “SIRT6,” “SIRT6,” “Sirt6,” “Sirt6” is meant to inclue the DNA, mRNA, ⁻m⁻RNA, cDNA, recombinant DNA or RNA, or the protein arising from the Sirtuin-6 gene. The human nucleotide sequence can be found at Gene ID: 51548. The mouse nucleotide sequence can be found at Gene ID: 50721.

By “neuronal” is meant to refer to and include any cells which compose the central or peripheral nervous system. See for details: Dowling JE, The retina an approachable part of the brain. Rev. ed. Cambridge, Mass.: Belknap Press of Harvard University Press; 2012.)

By “retinal” is meant to refer to and include any light-sensitive cells in the eye as well as the supporting cells that enable, facilitate, or are related to the phototransduction cascade.

By “nucleic acid” or “nucleic acid molecule” is meant to include a DNA, RNA, mRNA, cDNA, or recombinant DNA or RNA.

By “animal” is meant any member of the animal kingdom including vertebrates (e.g, frogs, salamanders, chickens, or horses) and invertebrates (e.g., worms, etc.). Preferred animals are mammals. Preferred mammalian animals include livestock animals (e.g., ungulates, such as bovines, buffalo, equines, ovines, porcines and caprines), as well as rodents (e.g., mice, hamsters, rats and guinea pigs), canines, felines and primates, By “non-human” is meant to include all animals, especially mammals and including primates other than human primates.

By “medium” or “media” is meant the nutrient solution in which cells and tissues are grown.

The term “pharmaceutically acceptable carrier”, as used herein means a pharmaceutically-acceptable material, composition or vehicle, such as a liquid or solid filler, diluent, excipient, solvent or encapsulating material, involved in carrying, or transporting a chemical agent. The diluent or carrier ingredients should, not be such as to diminish the therapeutic effects of the active cornpound(s).

The term “composition” as used herein means a product which results from the mixing or combining of more than one element or ingredient.

“Treating” or “treatment” of a state, disorder or condition includes:

(1) preventing or delaying the appearance of clinical symptoms of the state, disorder, or condition developing in a person who may be afflicted with or predisposed to the state, disorder or condition but does not yet experience or display clinical symptoms of the state, disorder or condition; or

(2) inhibiting the state, disorder or condition, i.e., arresting, reducing or delaying the development of the disease or a relapse thereof (in case of maintenance treatment) or at least one clinical symptom, sign, or test, thereof; or

(3) relieving the disease, i.e., causing regression of the state, disorder or condition or at least one of its clinical or sub-clinical symptoms or signs.

The benefit to a subject to be treated is either statistically significant or at least perceptible to the patient or to the physician.

A “therapeutically effective amount” means the amount of a compound that, when administered to an animal for treating a state, disorder or condition, is sufficient to effect such treatment. The “therapeutically effective amount” will vary depending on the compound, the disease and its severity and the age, weight, physical condition and responsiveness of the animal to be treated.

Acceptable excipients, diluents, and carriers for therapeutic use are well known in the pharmaceutical art, and are described, for example, in Remington: The Science and Practice of Pharmacy. Lippincott Williams & Wilkins (A. R. Ciennaro edit. 2005). The Choice of pharmaceutical excipient, diluent, and carrier can be selected with regard to the intended route of administration and standard pharmaceutical practice.

As used herein, the phrase “pharmaceutically acceptable” refers to molecular entities and compositions that are “generally regarded as safe” , e.g., that are physiologically tolerable and do not typically produce an allergic or similar untoward reaction, such as gastric upset, dizziness and the like, when administered to a human. Preferably, as used herein, the term “pharmaceutically acceptable” means approved by a regulatory agency of the Federal or a state government or listed in the U.S. Pharmacopoeia or other generally recognized pharmacopeias for use in animals, and more particularly in humans.

It is noted that Phorbol Myristate Acetate (PMA) acts as an inhibitor of Tsc1 and is described on the world wide web sabiosciences.com/iapp/mtor.html. Additional Tsc1 inhibitors are expected to be useful in aspects of the present invention.

Exemplary SIRT6 inhibitors have been identified and discussed by Yasuda et al. (Anal Chem. 2011 Oct. 1:,83(19):7400-71, Schlicker et al, (Aging, 2011 September; 3(9): 852-872), Singh et al, (J Chromatogr B Analyt Technol Biomed Life Sci. 2014 Oct 1; 0: 105-111), and Parenti et al. (J Med Chem. 2014 Jun 12;57(11):4796-804). Examples of specific SIRT6 inhibitors include fenugreek seed extract, quercetin, naringenin, vitexin. SYN17739303, BAS13555470, SYN10366754, and BAS00417531. Another example of a SIRT6 inhibitor is Vitexin (isolated from Hawthorn tree berries), which in certain instances can be given by PO or formulated in a sustained-release form, biodegradable implant in the human vitreous. Additionally, any suitable mode of delivery can be utilized for administering one or more of the SIRT6 inhibitors. These and additional exemplary SIRT6 inhibitors are expected to be useful alone, or in combination in aspects of the present invention.

In certain embodiments, the methods of the present disclosure can be used for arresting progression of or ameliorating vision loss associated with photoreceptor degeneration including retinitis pigmentosa (RP) and age-related macular degeneration (AMO) in the subject. Vision loss linked to retinitis pigmentosa may include decrease in peripheral vision, central (reading) vision, night vision, day vision, loss of color perception, loss of contrast sensitivity, or reduction in visual acuity. The methods of the present disclosure can also be used to prevent, or arrest photoreceptor function loss, or increase photoreceptor function in the subject.

RP is diagnosed in part, through an examination of the retina and genetic testing. The eye exam usually reveals abnormal, intraretinal pigment migration. Additional tests for diagnosing RP include electroretinogram (ERG) and visual field testing.

Methods for measuring or assessing visual function, retinal function (such as responsiveness to light stimulation), or retinal structure in a subject are well known to one of skill in the art. See. e.g. Kanski's Clinical Ophthalmology: A Systematic Approach. Edition 8, Elsevier Health Sciences, 2015. Methods for measuring or assessing retinal response to light include may include detecting an electrical response of the retina to a light stimulus. This response can be detected by measuring an electroretinogram (ERG; for example full-field ERG, muitifocal ERG, or ERG photostress test), visual evoked potential, or optokinetic nystagmus (see, e.g., Wester et al., Invest, Ophthalmol. mot Vis. Sci. 48:4542-4548, 2007). Furthermore, retinal response to light may be measured by directly detecting retinal response (for example by use of a microelectrode at the retinal surface). ERG has been extensively described by Vincent et al. Retina 2013 January;33(1)15-12. Thus, methods of the present disclosure can be used to improve visual retinal function (such as responsiveness to light stimulation), retinal structure, or any other clinical symptoms or phenotypic changes associated with ocular diseases in subjects afflicted with ocular disease.

“Patient” or “subject” refers to mammals and includes human and veterinary subjects.

The dosage of the therapeutic formulation will vary widely, depending upon the nature of the disease, the patient's medical history, the frequency of administration, the manner of administration, the clearance of the agent from the host, and the like. The initial dose may be larger, followed by smaller maintenance doses. The dose may be administered as infrequently as weekly or biweekly, or fractionated into smaller doses and administered daily, semi-weekly, etc., to maintain an effective dosage level. In some cases, oral administration will require a higher dose than if administered intravenously. In some cases, topical administration will include application several times a day, as needed, for a number of days or weeks in order to provide an effective topical dose.

The term “carrier” refers to a diluent, adjuvant, excipient, or vehicle with which the compound is administered. Such pharmaceutical carriers can be sterile liquids, such as water m and oils, including those of petroleum, animal, vegetable or synthetic origin, such as peanut oil, soybean oil, mineral oil, olive oil, sesame oil and the like. Water or aqueous solution, saline solutions and aqueous dextrose and glycerol solutions are preferably employed as carriers, particularly for injectable solutions. Alternatively, the carrier can be a solid dosage form carrier, including but not limited to one or more of a binder (for compressed pills), a glidant, an encapsulating agent, a flavorant, and a colorant. Suitable pharmaceutical carriers are described in “Remington's Pharmaceutical Sciences” by E. W. Martin.

As used herein, the term “adjuvant” refers to a compound or mixture that enhances the immune response to an antigen. An adjuvant can serve as a tissue depot that slowly releases the antigen and also as a lymphoid system activator that non-specifically enhances the immune response (Hood et al. Immunology, Second Ed., 1984, Benjamin/Cummings: Menlo Park, Calif, p. 384). Often, a primary challenge with an antigen alone, in the absence of an adjuvant, will fail to elicit a humoral or cellular immune response, Adjuvants include, but are not limited to, complete Freund's adjuvant, incomplete Freund's adjuvant, saponin, mineral gels such as aluminum hydroxide, surface active substances such as lysolecithin, plutonic polyols, polyanions, peptides, oil or hydrocarbon emulsions, keyhole limpet hemocyanins, and potentially useful human adjuvants such as N-acetyl-muramyl-L-threonyl-D-isoglutamine (thr-MDP), N-acetyl-nor-muramyl-L-alanyl-D-isoglutamine, N-acetyl uramylmuramyl-L-alanyl-D-isoglutaminyl-L-alanine-2)1′-2′-dipalmitoyl-sn-glycero-3-hydroxyphosphoryloxy)-ethylamine, and BCG (bacille Calmette-Guerin). Preferably, the adjuvant is pharmaceutically acceptable.

Vectors of the present disclosure can comprise any of a number of promoters known to the art, wherein the promoter is constituitive, regulatable or inducible, cell type specific, tissue-specific, or species specific. In addition to the sequence sufficient to direct transcription, a promoter sequence of the invention can also include sequences of other regulatory elements that are nvolved its modulating transcription (e.g.: enhancers, kozak sequences and introns). Many promoter/regulatory sequences useful for driving constitutive expression of a gene are available in the art and include, but are not limited to, for example, CMV (cytomegalovirus promoter),EF1a (human beta-actin promoter), SV40 (simian vacuolating virus 40 promoter), PGK (mammalian phosphoglycerate kinase promoter), Ubc (human ubiquitin: C promoter), human beta-actin promoter, rodent beta-actin promoter, CBh (chicken beta-actin promoter), CAG (hybrid promoter contains CMV enhancer, chicken beta actin promoter, and (rabbit beta-globin splice acceptor), TRE (Tetracycline response element promoter), HI (human polymerase HI RNA promoter), U6 (human U6 small nuclear promoter), and the like. Moreover, inducible and tissue specific expression of an RNA, transmembrane proteins, or other proteins can be accomplished by placing the nucleic acid encoding such a molecule under the control of an inducible or tissue specific promoter/regulatory sequence. Examples of tissue, specific or inducible promotor/regulatory sequences which are useful for this purpose include, but are not limited to, the rhodopsin promoter, MMTV LTR inducible promoter, the SV40 late enhancer/promoter, synapsin 1 promoter, ET hepatocyte promoter, GS glutamine synthase promoter and many others. Various commercially available ubiquitous as well as tissue-specific promoters can be found at http://www.invivogen.com/prom-a-list and https://www.addgene.org/ In addition, promoters which are well known in the art can be induced in response to inducing agents such as metals, glucocorticoids, tetracycline, hormones, and the like, are also contemplated for use with the invention. Thus, it will be appreciated that the present disclosure includes the use of any promoter/regulatory sequence known in the art that is capable of driving expression of the desired protein operably linked thereto,

Vectors according to the present disclosure can be transformed, transfected or, otherwise introduced into a wide variety of host cells. Transfection refers to the taking up of a vector by a host cell whether or not any coding sequences are in fact expressed. Numerous methods of transfection are known to the ordinarily skilled artisan, for example, lipofectamine calcium phosphate co-precipitation, electroporation, DEAE-dextran treatment, microinjection, viral transduction, and other methods known in the art. Transduction refers to entry of a virus into the cell and expression (e.g., transcription and/or translation) of sequences delivered by the viral vector genome. In the case of a recombinant vector, “transduction” generally refers to entry of the recombinant viral vector into the cell and expression of a nucleic acid of interest delivered by the vector genome.

In certain embodiments, the methods described herein can be utilized to treat ocular disease, neuronal disease, or improve photoreceptor function in a patient and can comprise administering to the patient an effective concentration of a composition comprising any of the recombinant AAV's described herein and a pharmaceutically acceptable carrier. In one embodiment, an effective concentration of virus is 1×10⁶−11×10¹³ GC/ml. The range of viral concentration effective for the treatment can vary depending on factors including, but not limited to specific mutation, patient's age, and other clinical parameters.

Production of recombinant AAV vectors and their use in in vitro and in vivo administration has been discussed in detail by Gray et al. (Curr Protoc Neurosci. 2011 October, Chapter: Unit 4.17).

The recombinant AAV containing the desired recombinant DNA can be formulated into a. pharmaceutical composition intended for subretinal or intravitreal injection. Such formulation involves the use of a pharmaceutically and/or physiologically acceptable vehicle or carrier, particularly one suitable for administration to the eye, e.g., by subretinal injection, such buffered saline or other buffers, e.g,, HEPES, to maintain at appropriate physiological levels, and, optionally, other medicinal agents, pharmaceutical agents, stabilizing agents, buffers, carriers, adjuvants, diluents, etc. For injection, the carrier will typically be a liquid. Exemplary physiologically acceptable carriers include sterile, pyrogen-free water and sterile, pyrogen-free, phosphate buffered saline.

In one embodiment, the carrier is an isotonic sodium chloride solution. In another embodiment, the carrier is balanced salt solution. In one embodiment, the carrier includes tween. If the virus is to be stored long-term, it may be frozen in the presence of glycerol or Tween-20. In another embodiment, the pharmaceutically acceptable carrier comprises a surfactant, such as, perfluorooctane (Perfluoron liquid). In certain embodiments, the pharmaceutical composition described above is administered to the subject by subretinal injection. In other embodiments, the pharmaceutical composition is administered by intravitreal injection. Other forms of administration that may be useful in the methods described herein include, but are not limited to, direct delivery to a desired organ (e.g., the eye), oral, inhalation, intranasal, intratracheal, intravenous, intramuscular, subcutaneous, intradermal, and other parental routes of administration. Additionally, routes of administration may be combined, if desired.

In preferred embodiments, route of administration is subretinal injection or intravitreal injection.

“Treat” or “treating” refers to administering a therapeutic agent, such as a composition containing any of the tissue-specific, e.g., neuronal or ocular targeted viral vectors, RNAi, shRNA or other Tsc1 or Sirt6 inhibitors, combinations thereof, or similar compositions described herein, internally or externally to a subject or patient having one or more disease symptoms, or being suspected of having a disease or being at elevated at risk of acquiring a disease, for which the agent has therapeutic activity. Gene editing technology such as CRISPR/cas9 methods may also be utilized to carry out tissue-specific reduction of Tsc1, Sirt6 or a combination thereof. Typically, the agent is administered in an amount effective to alleviate one or more disease symptoms in the treated subject or population, whether by inducing the regression of or inhibiting, the progression of such symptom(s) by any clinically measurable degree. The amount of a therapeutic agent that is effective to alleviate any particular disease symptom (also referred to as the “therapeutically effective amount”) may vary according to factors such as the disease state, age, and weight of the patient, and the ability of the drug to elicit a desired response in subject. Whether a disease symptom has been alleviated can be assessed by any clinical measurement typically used by physicians or other skilled healthcare providers to assess the severity or progression status of that symptom. While an embodiment of the present invention (e.g., a treatment method or article of manufacture) may not be effective in alleviating the target disease symptom(s) in every subject, it should alleviate the target disease symptom(s) in a statistically significant number of subjects as determined by any statistical test known in the art such as the Student's t-test, the chi²-test, the U-test according to Mann and Whitney, the Kruskal-Wallis test (H-test), Jonckheere-Teapstra-test and the Wilcoxon-test.

“Treatment,” as it applies to a human, veterinary, or research subject, refers to therapeutic treatment, prophylactic or preventative measures, to research and diagnostic applications. “Treatment” as it applies to human, veterinary, or research subject, or cell, tissue, or organ, encompasses transfection of any of the tissue-targeted viral vectors, delivery of RNAi, shRNA or other TSC1 or SIRT6 inhibitors, combinations thereof, or similar compositions, including gene editing technology such as CRISPR/cas9 methods, which may be utilized to carry out tissue specific reduction of TSC1 or SIRT6, combinations thereof or related methods described herein as applied to a human or animal subject, a cell, tissue, physiological compartment, or physiological fluid.

### Inhibitory Nucleic Acids that Hybridize to Tsc1 or Sirt6

It is noted that in addition to Tsc1, the transcriptional repressor of glycolysis known as Sirtuin 6 (Sirt6 ) induced degeneration and is another process which could potentially be blocked by inhibitory compounds in a similar manner as described herein for Tsc1 . Any number of means for inhibiting Tsc1 and/or Sirt6 activity or gene expression can be used in the methods of the invention. For example, a nucleic acid molecule complementary to at least portion of a human Tsc1 and/or Sirt6 encoding nucleic acid can be used to inhibit Tsc1 and/or Sirt6 gene expression. Means for inhibiting gene expression using short RNA molecules, for example, are known. Among these are short interfering RNA (sIRNA), small temporal RNAs (stRNAs), and micro-RNAs (miRNAs) Short interfering RNAs silence gene, through a mRNA ,gradation pathway, while stRNAs and miRNAs are approximately 21 or 22 in RNAs that in e processed from endogenously encoded hairpin-structured precursors, and function to silence genes via translational repression See, e.g., McManus et al., RNA, 8(6);842-50(2002)Morris et al., Science, 305(5681);1289-92(2004); He and Hannon, Nat ReV Genet. 5(7):522-31 (2004).

“RNA. interference, or RNAi” a form of post-transcriptional gene silencing (“PTGS”), describes effects that result from the introduction of double-stranded RNA into cells (reviewed in Fire, A. Trends Genet 15:358-363 (1999); Sharp, P. Genes Dev 13;139-141 (1999); Hunter, C. Curr Biol 9:R440-R442 (1999); Balcombe. D. Curr Biol. 9:R599-R601 (1999); Vaucheret et al. Plant J 16: 651-659 (1998)). RNA interference, commonly referred to as RNAi, offers a way of specifically inactivating a cloned gene, and is a powerful tool for investigating gene function.

The active anent in RNAi is a long double-stranded (antiparallel duplex) RNA, with one of the strands corresponding or complementary to the RNA which is to he inhibited. The inhibited RNA, is the target RNA. The long double stranded RNA is chopped into smaller duplexes of approximately 20 to 25 nucleotide pairs, after which the mechanism by which the smaller RNAs inhibit expression of the target is largely unknown at this time. While RNAi was shown initially to work well in lower eukaryotes, for mammalian cells, it was thought that RNAi might be suitable only for studies on the oocyte and the preimplantation embryo.

More recently, it was shown that RNAi would work in human cells if the RNA strands were provided as pre-sized duplexes of about 19 nucleotide pairs, and RNAi worked particularly well with small unpaired 3′ extensions on the end of each strand (Elbashir et al. Nature 411: 494-498 (2001)). In this report, “short interfering RNA” (siRNA, also referred to as small interfering RNA) were applied to cultured cells by transfection in oligofectamine micelles. These RNA duplexes were too short to elicit sequence-nonspecific responses like apoptosis, yet they efficiently initiated RNAi. Many laboratories then tested the use of siRNA to knock out target genes in mammalian cells. The results demonstrated that siRNA works quite well in most instances.

Software programs for predicting siRNA sequences to inhibit the expression of a target protein are commercially available and find use. One program, siDESIGN from Dharmacon, Inc, (Lafayette, Colo.), permits predicting siRNAs for any nucleic acid sequence, and is available on the interact at dharmacan.com. Programs for designing siRNAs are also available from others, including Genscript (available on the interact tat genscript,com/ssl-bin/app/rnai) and, to academic and non-profit researchers, from the Whitehead Institute for Biomedical Research found on the worldwide web at “jura.wi.mit.edu/pubint/http://iona.wi.mit.edu/siRNAext/.”

Any suitable viral lockdown system could be utilized for decreasing Tsc1 and/or Sirt6 mRNA levels including AAV, lentiviral vectors, or other suitable vectors that are capable of being targeted specifically to the liver: (See Zuckerman and Davis (2015),

Additionally, specifically targeted delivery of shTsc1 or Sirt6 mRNA or other Tsc1 or Sirt6 blocking molecule (nucleic acid, peptide, or small molecule) could be delivered by targeted liposome, nanoparticle or other suitable means.

As described herein we provide methods as well as one or more agents/compounds that silence or inhibit Tsc1, Sirt6, or combinations thereof for the treatment, prophylaxis or alleviation of degenerative eye conditions including RP AMD, glaucoma, and related conditions, as well as neurodeuenerative conditions described herein, or predisposition to such conditions.

RNA interference (RNAi) is a method of post transcriptional gene silencing (PTGS) induced by the direct introduction of double-stranded RNA (dsRNA) and has emerged as a useful tool to knock out expression of specific genes in a variety of organisms. RNAi is described by Fire et al., Nature 391:806-811 (1998), Other methods of PTGS are known and include, for example, introduction of a transgene or virus. Generally, in PTGS, the transcript of the silenced gene is synthesised but does not accumulate because it is rapidly degraded. Methods for PTGS, including RNAi are described, for example, in the Ambion.com world wide web site, in the directory “/hottopics/”, in the “mai” file.

Suitable methods for RNAi in vitro are described herein. One such method involves the introduction of siRNA (small interfering RNA). Current models indicate that these 21-23 nucleotide dsRNAs can induce PTGS. Methods for designing effective siRNAs are described, for example, in the Ambion web site described above. RNA precursors such as Short Hairpin RNAs (shRNAs) can also be encoded by all, or a part of the Tsc1 or Sirt6 nucleic acid sequence.

Alternatively, double-stranded (ds) RNA is a powerful way of interfering with gene expression in a range of organisms that has recently been shown to be successful in mammals (Wianny and Zernicka-Goetz, 2000, Nat Cell Biol 2:70-75). Double stranded RNA corresponding to the sequence of a Tsc1 or Sirt6 polynucleotide can be introduced into or expressed in oocytes and cells of a candidate organism to interfere with Tsc1 and/or Sirt6 activity.

Tsc1 and/or Sirt6 gene expression may also be modulated by introducing peptides or small molecules which inhibit gene expression or functional activity. Thus, compounds identified by the assays described herein as binding to or modulating, such, as down-regulating, the amount, activity or expression of TSC1 and/or SIRT6 polypeptide may be administered to target cells to prevent the function of TSC1 and/or SIRT6 polypeptide. Such a compound may be administered along with a pharmaceutically acceptable carrier in an amount effective to down-regulate expression or activity TSC1 and/or SIRT6, or by activating or down-regulating a second signal which controls STC1 and/or SIRT6 expression, activity or amount, and thereby alleviating the abnormal condition.

Alternatively, gene therapy may be employed to control the endogenous production of Tsc1 and/or Sirt6 by the relevant cells such as neuronal cells or photoreceptor cells, ie., rod and cone cells in the subject. For example, a polynucleotide encoding a Tsc1 or Sirt6 siRNA or a portion, of this may be engineered for expression in a replication defective retroviral vector, as discussed below. The retroviral expression construct may then be isolated and introduced into a packaging cell transduced with a retroviral plasmid vector containing RNA encoding an anti-Tsc1 or Sirt6 siRNA such that the packaging cell now produces infectious viral particles containing the sequence of interest. These producer cells may be administered to a subject for engineering cells in vivo and regulating expression of the TSC1 or SIRT6 polypeptide in vivo. For overview of gene therapy, see Chapter 20, Gene Therapy and other Molecular Genetic-based Therapeutic Approaches, (and references cited therein) in Human Molecular Genetics, T Strachan and A P Read, BIOS Scientific Publishers Ltd (1996).

In some embodiments, the level of Tsc1, Sirt6, or combinations thereof, is decreased in a desired target cell such as a neuronal cell or the vitreous. Furthermore, in such embodiments, treatment may be targeted to, or specific to. desired target cell such as a neuronal cell or the vitreous. The expression of Tsc1 or Sirt6 may be specifically decreased only in the desired target cell such as a neuronal cell or the vitreous (i.e. those cells which are predisposed to the condition, or exhibiting the disease already), and not substantially in other non-diseased cells. In these methods, expression of TSC1 and/or SIRT6 may not be substantially reduced in other cells, i.e., cells which are not desired target cells. Thus, in such, embodiments, the level of TSC1 or combinations thereof, remains substantially the same or similar in non-target cells in the course of or following treatment.

Alternately, one may administer the viral vectors, RNAi, shRNA or other Tsc1 or Sirt6 inhibitors, or related compounds in a local rather than systemic manner, for example, via injection of directly into the desired target site, often, in a depot or sustained release formulation. Furthermore, one may administer the composition in a targeted drug delivery system, for example, in a liposome coated with a tissue-specific antibody, targeting, for example, specific neurons, or the virtreous, and more specifically hepatocytes. The liposomes will be targeted to and taken up selectively by the desired tissue. Also included in a targeted drug delivery system is nanoparticle specific delivery of the viral vectors, RNAi, shRNA or other Tsc1 Sirt6 inhibitors, alone or in combination. A summary of various delivery methods and techniques of siRNA administration in ongoing clinical trials is provided in Zuckerman and Davis 2015; Nature Rev. Drug Discovery, Vol. 14: 843-856, December, 2015.

The administration regimen depends on several factors, including the serum or tissue turnover rate of the therapeutic composition, the level of symptoms, and the accessibility of the target cells in the biological matrix. Preferably, the administration regimen delivers sufficient therapeutic composition to effect improvement in the target disease state, while simultaneously minimizing undesired side effects. Accordingly, the amount of biologic delivered depends in part on the particular therapeutic composition and the severity of the condition being treated.

In preferred embodiments, route of administration is subretinal injection or intravitreal injection.

Methods for modification of genomic DNA are well known in the art. For example, methods may use a DNA digesting agent to modify the DNA by either the non-homologous end joining DNA repair pathway (NHEJ) or the homology directed repair (HDR) pathway. The term “DNA digesting agent” refers to an agent that is capable of cleaving bonds (i.e. phosphodiester bonds) between the nucleotide subunits of nucleic acids.

in one embodiment, the DNA digesting agent is a nuclease. Nucleases are enzymes that hydrolyze nucleic acids. Nucleases may be classified as endonucleases or exonucleases. An endonuclease is any of a group of enzymes that catalyze the hydrolysis of bonds between nucleic acids in the interior of a DNA or RNA molecule. An exonuclease is any of a group of enzymes that catalyze the hydrolysis of single nucleotides from the end of a DNA or RNA chain. Nucleases may also be classified based on whether they specifically digest DNA or RNA. A nuclease that specifically catalyzes the hydrolysis of DNA may be referred to as a deoxyribonuclease or DNase, whereas a nuclease that specifically catalyses the hydrolysis of RNA may be referred to as a ribonuclase or an RNase. Some nucleases are specific to either single-stranded or double-stranded nucleic acid sequences. Same enzymes have both exonuclease and endonuclease properties. In addition, some enzymes are able to digest both DNA and RNA sequences.

Non-limiting examples of the endonucleases include a zinc finger nuclease (ZFN), a ZFN dimer, to ZFNickase, a transcription activator-like nuclease (TALEN), or a RNA-guided DNA endonuclease (e.g., CRISPR/Cas9). Meganucleases are endonucleases characterized by their capacity to recognize and cut large DNA sequences (12 base pairs or greater) Any suitable meganuclease may be used in the present methods to create double-strand breaks in the host genome, including endonucleases is the LAGLIDADG (SEQ ID NO:1) and PI-Sce family.

One example of a sequence-specific nuclease system that can be used with the methods and compositions described herein includes the CRISPR system (Wiedenheft, B. et al. Nature 482, 331-338 (2012) Jinek, M. et al. Science 337, 816-821 (2012); Mali, P. et al. Science 339, 823-826 (2013); Cong, L. et al. Science 339, 819823 (2013)). The CRISPR (Clustered Regularly interspaced Short Palindromic Repeats) system exploits RNA-guided DNA-binding and sequence-specific of target DNA. The guide RNA/Cas combination confers site specificity to the nuclease. A single guide RNA (sgRNA) contains about 20 nucleotides that are complementary a target genomic DNA sequence upstream of a genomic PAM (protospacer adjacent motifs) site (NCG) and a constant RNA scaffold region. The Cas (CRISPR-associated) protein binds to the sgRNA and the target DNA to which the sgRNA binds and introduces a double-strand break in a defined location upstream of the PAM site. Cas9 harbors two independent domains homologous to HNH and RuvC endonucleases, and by mutating either of the two domains, the Cas9 protein can be convened to a nickase that introduces single strand breaks (Cong, L. et al. Science 339, 819-823 (2013). It is specifically contemplated that the methods and compositions of the present disclosure can be used with the single- or double-strand-inducing version of Cas9, as well as with other RNA-guided DNA nucleases, such as other bacterial Cas9-like systems. The sequence-specific nuclease of the present methods and compositions described herein can be engineered, chimeric, or isolated from an organism. The nuclease can be introduced into the cell in form of a DNA, mRNA and protein. The applications of the CRISPR/Cas system to inhibiting or downregulating Tsc1 or Sirt6 are easily adapted, and the following guide RNAs have been developed for use in acquiring preliminary a on this subject: Tsc1—GAGGATGTCGGTGAATTACG (SEQ ID No:2); Sirt6—GAGGATGTCGGTGAATTACG (SEQ ID NO:2).

In one embodiment, the methods of the present disclosure comprise using one or more sgRNAs to “Chop,” remove, or suppress glycosis regulators Tsc1 or Sirt6. In another embodiment, one sgRNA(s) is used to “Chop”, remove, or suppress glycosis regulators, Tsc1 or Sirt6. disease-related gene. In yet further embodiment, two or more sgRNA(s) are used to “Chop”, remove, or suppress an autosomal dominant disease-related gene.

In one embodiment, the DNA digesting agent can be a site-specific nuclease. In another embodiment, the site-specific nuclease may be a Cas-family nuclease. In a more specific embodiment, the Cas nuclease may be a Cas9 nuclease.

In one embodiment, Cas protein may be a functional derivative of a naturally occurring Cas protein.

In addition to well characterized CRISPR-Cas system, a new CRISPR enzyme, called Cpf1 protein 1 of PreFran subtype) has recently been described (Zetsche et al. Cell. pii: S0092-8674(15)01200-3. doi: 10.1016/j.cell. 2015.09 038 (2015)). Cpf1 single RNA-guided endonuclease that lacks tracrRNA, and utilizes a T-rich protospacer-adjacent motif. The authors demonstrated that Cpf1 mediates strong DNA interference with characteristics distinct from those of Cas9. Thus, in one embodiment of the present invention, CRISPR-Cpf1 system can be used to cleave a desired region within the targeted gene.

In further embodiment, the DNA digesting agent is a transcription activator-like effector nuclease (TALEN). TALENs are composed of a TAL effector domain that binds to a specific nucleotide sequence and an endonuclease domain that catalyzes a double strand break at the target site (PCT Patent Publication No. WO2011072246; Miller et al., Nat. Biotechnol, 29, 143-148 (2011); Cermak et al. Nucleic Acid Res. 39, e82 (2011). Sequence-specific endonucleases may be modular in nature, and DNA binding specificity is obtained by arranging one or more modules. Bibikova et al., Mol. Cell. Biol. 21, 289-297 (2001), Boch et al., Science 326, 1509-1512 (2009).

ZFNs can be composed of two or more (e.g., 2-8, 3-6, 6-8, or more) sequence-specific DNA binding domains (e.g., zinc finger domains) fused to an effector endonuclease domain (e.g., the FokI endonuclease). Porteus et al., Nat. Biotechnol. 23, 967-973 (2005). Kim et al. (2007) Hybrid enzymes: Zinc finger fusions to Fok I cleavage domain, Proceedings of the National Academy of Sciences of USA, 93: 1156-1160, U.S. Pat. No. 6,824,978, PCT Publication Nos. WO1995/09233 and WO01994018313.

In one embodiment, the DNA digesting agent is a site-specific nuclease of the group or selected from the group consisting of omega, zinc finger, TALE, and CRISPR/Cas.

The sequence-specific endonuclease of the methods and compositions described here can be engineered, chimeric, or isolated from organism. Endonucleases can be engineered to recognize a specific DNA sequence, by, e.g., mutagenesis. Seligman et al., (2002) Mutations altering the cleavage specificity of homing endonuclease, Nucleic Acids Research 30: 3870-3879. Combinatorial assembly is a method where protein subunits form different enzymes can he associated or fused. Amould et al. (2006) Engineering of large numbers of highly specific homing endonucleases that induce recombination to novel DNA targets. Journal of Molecular Biology 355: 443-458. In certain embodiments, these two approaches, mutagenesis and combinatorial assembly, can be combined to produce an engineered endonuclease with desired DNA recognition sequence.

The sequence-specific nuclease can be introduced into the cell in the form of a protein or in the form of nucleic acid encoding the sequence-specific nuclease, such as an mRNA or a cDNA. Nucleic acids can be delivered as part of a larger construct, such as a plasmid or viral vector, or directly, e.g., by electroporation, lipid vesicles, viral transporters, microinjection, and biolistics. Similarly, the construct containing one or more transgenes can be delivered by any method appropriate for introducing nucleic acids into a cell.

Single guide RNA(s) used in the methods of the present disclosure can be designed so that they direct binding of the Cas-sgRNA complexes to pre-determined cleavage sites in a genome. In one embodiment, the cleavage sites may be chosen so as to release a fragment or sequence that contains a region of autosomal dominant disease-related gene. In further embodiment, the cleavage sites may be chosen so as to release a fragment or sequence that contains a region of genes encoding glycosis regulators, Tsc1 or Sirt6.

For Cas family enzyme (such as Cas9) to successfully bind to DNA, the target sequence in the genomic DNA should be complementary to the sgRNA sequence and must be immediately followed by the correct protospacer adjacent motif or “PAM” sequence. “Complementarity” refers to the ability of a nucleic acid to form hydrogen bond(s) with another nucleic acid sequence by either traditional Watson-Crick or other traditional types. A percent complementarity indicates the percentage of residues in a nucleic acid molecule, which can form hydrogen bonds (e.g., Watson-Crick base pairing) with a second nucleic acid sequence. Full complementarity is necessarily required, provided there is sufficient complementarity to cause hybridization and promote formation of a CRISPR complex. A target sequence may comprise any polynucleotide, such as DNA or RNA polynucleotides. The Cas9 protein can, tolerate mismatches distal from the PAM, however, mismatches within the 12 base pairs (bps) of sequence next to the PAM sequence can dramatically decrease the targeting efficiency. The PAM sequence is present in the DNA target sequence but not in the sgRNA sequence. Any DNA sequence with the correct target sequence followed by the PAM sequence will be bound by Cas9. The PAM sequence varies by the species of the bacteria from which Cas9 was derived. The most widely used CRISPR system is derived from S. pyogenes and the PAM sequence is NGG located on the immediate 3′ end of the sgRNA recognition sequence. The PAM sequences of CRISPR systems from exemplary bacterial species include: Streptococcus pyogenes (NGG), Neisseria meningitidis (NNNNGATT), Streptococcus thermophilus (NNAGAA) and Treponemo denticolo (NAAAAC).

sgRNA(s) used in the present disclosure can be between about 5 and 100 nucleotides long, or longer (e.g., 5, 6, 7, 8, 9, 10, 11, 12. 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 63, 64, 65, 66, 67, 68, 69, 70, 71, 72, 73, 74, 75, 76, 77, 78, 79, 80, 81, 82, 83, 84, 85, 86, 87, 88, 89, 90, 91 92, 93, 94, 95, 96, 97, 98, 99, or 100 nucleotides in length, or longer). In one embodiment, sgRNA(s) can be between about 15 and about 30 nucleotides in length (e.g., about 15-26, 15-26, 15-25; 16-30, 16-29, 16-26, 16-25; or about 18-30, 18-29, 18-26, or 18-25 nucleotides in length).

To facilitate sgRNA design, many computational tools have been developed (See Plykhozhij et al. (PLoS ONE, 10(3): (2015)), Zhu et al. (PLoS ONE 9(9) (2014)); Xiao et. al., (Bioinformatics. January 21 (2014)); Heigwer et al., (Nat Methods, 11(2): 122-123 (2014)). Methods and tools for guide RNA design are discussed by Zhu (Frontiers in Biology, 10 (4) pp 289-296 (2015)), which is incorporated by reference herein. Additionally, there is a publically available software tool that can be used to facilitate the design of sgRNA(s) (http://www.genscript.com/gRNA-design-tool.html).

### Kits

In another aspect, the present invention provides any of the compositions described herein in kits, optionally including instructions for use of the compositions (e.g., for improving neuronal survival and/or inhibiting SIRT6 and or TSC1), That is, the kit can include a description of use of a composition in any method described herein. A “kit,” as used herein, typically defines a package, assembly, or container (such as an insulated container) including one or more of the components or embodiments of the invention, and/or other components associated with the invention, for example, as previously described. Each of the components of the kit may be provided in liquid form (e.g., in solution), or in solid form (e.g., a dried powder, frozen, etc.).

In some cases, the kit includes one or more components, which may be within the same or in two or more receptacles, and/or in any combination thereof. The receptacle is able to contain a liquid, and non-limiting examples include bottles, vials, jars, tubes, flasks, beakers, or the like. In some cases, the receptacle is spill-proof (when closed, liquid cannot exit the receptacle, regardless of orientation of the receptacle).

Examples of other compositions or components associated with the invention include, but are not limited to, diluents, salts, buffers, chelating agents, preservatives, drying agents, antimicrobials, needles, syringes, packaging materials, tubes, bottles, flasks, beakers, and the like, for example, for using, modifying, assembling, storing, packaging, preparing, mixing, diluting, and/or preserving the components for a particular use. In embodiments where liquid forms of any of the components are used, the liquid form may be concentrated or ready to use.

A kit of the invention generally will include instructions or instructions to a website or other source in any form that are provided for using the kit in connection with the components and/or methods of the invention. For instance, the instructions may include instructions for the use, modification, mixing, diluting, preserving, assembly, storage, packaging, and/or preparation of the components and/or other components associated with the kit. In some cases, the instructions may also include instructions for the delivery of the components, for example, for shipping at room temperature, sub-zero temperatures, cryogenic temperatures, etc. The instructions may be provided in any form that is useful to the user of the kit, such as written or oral (e,g, telephonic), digital, optical, visual (e.g., videotape, DVD, etc.) and/or electronic communications (including Internet or web-based communications), provided in any manner.

As used herein, instructions can include protocols, directions, guides, warnings, labels, notes, and/or “frequently asked questions” (FAQs), and typically involve written instructions on or associated with the invention and/or with the packaging of the invention. Instructions can also include instructional communications in any form (e.g., oral, electronic, digital, optical, visual, etc.), provided in any manner (e.g., within or separate from a kit) such that a user will clearly recognize that the instructions are to be used with the kit.

### EXAMPLES

### Example 1

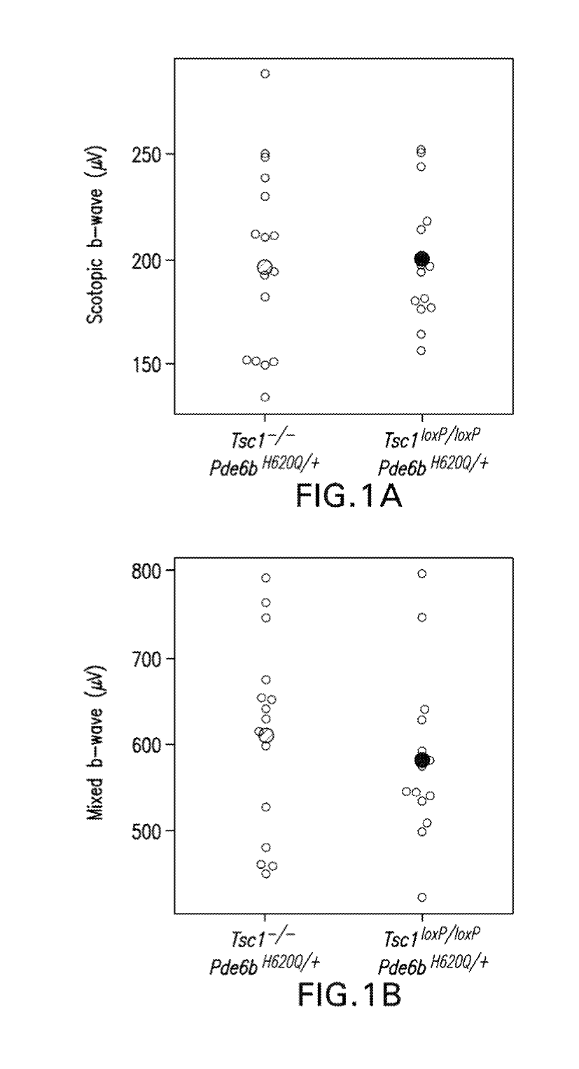

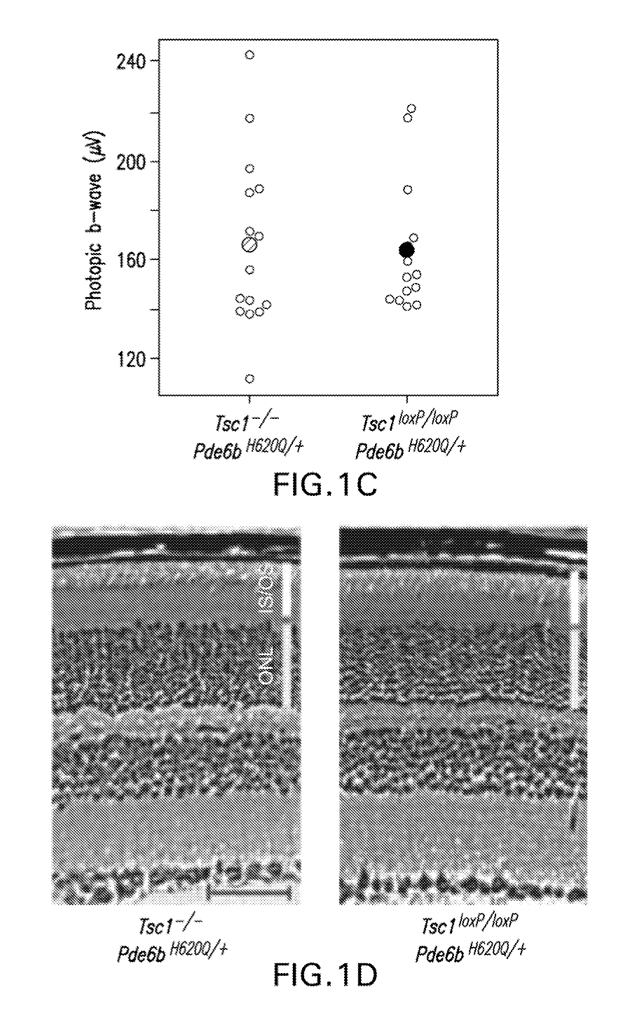

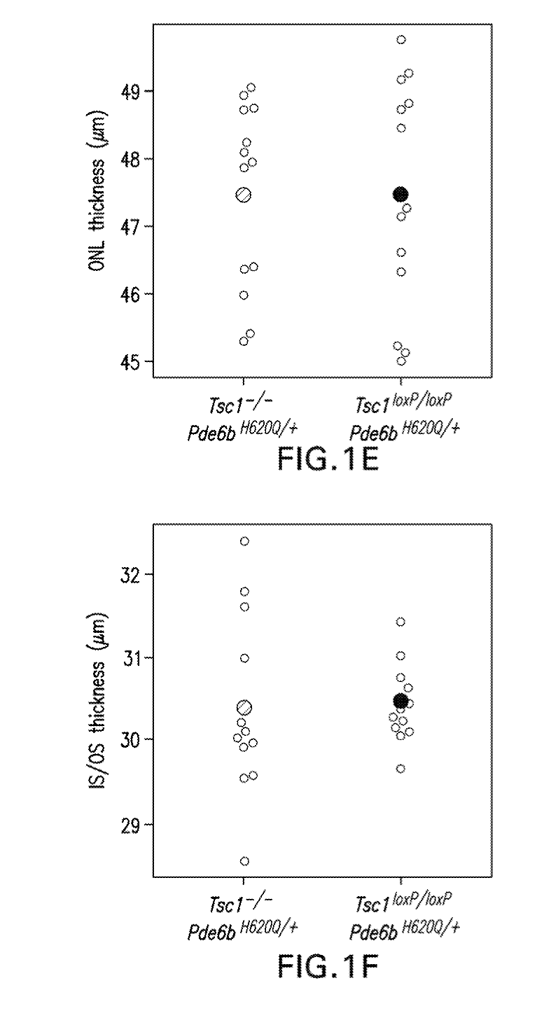

Effects TSC1 deficiency in [symbol] heterozygotes. To determine whether Tsc1 knockout in phenotypically wild type affects retia morphology or function, we generated a tamoxifen-inducible, rod-specific Cre recombinase mouse line by crossing Tsc1^{tm1Djk/J}, [symbol] , and Pde6g mice. Following intraperitoneal (IP) injection of tamoxifen, exons 17 and 18 of the Tsc1 gene was excised, leaving the mice with a nonfunctional Tsc1 protein, one functional copy of Pde6b, and the genotype, Tsc1^{-/-} [symbol] Pde6^{CreERT2}. Control mice were injected with oil instead of tamoxifen and thus had the genotype, Tsc1^{loxP/loxP} [symbol] Pde6g^{CreERT2}. Five weeks follow injection, electroretinogram (ERG) recordings of scotopic, photopic,and mixed rod-cone magnitudes were acquired and revealed no statistically significant difference between the groups (FIG. 1A-C). Retinae were harvested and exposed to H&E staining, revealing comparable morphology of the outer nuclear layer (ONL), inner segment(IS), and OS in the control and experimental groups' retinae (FIG. 1A). These findings suggest that Cre induction and Tsc1 knockout do not affect the function or morphology of wild type mouse retinae.

B. Anatomical rescue in [symbol] homozygotes lacking TSC1 at early and late stages. Subsequently second line of tamoxifen-inducible Tsc1 knockout mice was generated that was homozygous at the Pdeb6locus for the H620Q point mutation. Following tamoxifen injection, the experimental group, hereafter referred to as treated, experimental, knockout, or Tsc1^{-/-} [symbol] had complete ablation of Tsc1 rods, while the control group, denoted control or Tsc1^{loxP/loxP} [symbol] , had normal Tsc1expression. Histology was performed weekly and revealed a more robust and healthy ONL in Tsc1^{-/-} [symbol] at early time points (weeks 1-2 post compared to Tsc1^{loxP/loxP} [symbol] mice as measured by ONL nuclei density and width (FIG. 2). ONL cell density and thickness were statistically greater in he experimental group at one and weeks after injection compared to controls, although in both groups, a decrease was observed over time. Conversely, the IS/OS layer width increased over time, as would be expected according to physiological photoreceptor development (22, 23) Notably, the Tsc^{-/-} [symbol] mice IS/OS layer width was thicker at each timepoint compared to that of the control mice (FIG. 2B). The predicted trend line for the ONL nuclei density and width was negative for both groups but steeper for the control group. Conversely, the predicted trend line was positive and steeper for the IS/OS layer thickness in the Tsc2^{-/-} [symbol] experimental mice compared to controls. However, this diminished over time (FIG. 3). Histology revealed decreasing thickness both groups' ONL and IS/OS layers at later time points, although the width of the layers in the knockout mice remained noticeably thicker even at final time point (FIG. 3 ). Unexpectedly, trend lines mapping ONL nuclei density and width were steeper in the treated group compared to the control mice, which opposes findings obtained in the early stages following IP injection (FIG. 3B). Another distinguishing characteristic of the late stage (4-10 weeks) compared with the early stage is that the IS/OS layer thickness diminished over time in both groups, while in the early stage it had been increased with time. The predicted trend line between the groups was parallel, although the Tsc1^{-/-} [symbol] group had an overall higher mean thickness at each time point.

C. Functional rescue of photoreceptors in [symbol] homozygotes lacking TSC1. Functional assay of photoreceptor vitality was determined by scotopic, photopic, and mixed ERG recordings at biweekly intervals from 4-12 weeks (FIG. 4A). A decline in the recording amplitudes over time was evident even in the experimental group, but the average magnitude of scotopic, photopic, and mixed ERG recordings was statistically higher at every time point in Tsc^{-/-} [symbol] compared to Tsc1^{loxP/loxP} [symbol] mice. By 12 weeks, the ERG was no longer detectable in the control mice, while the experimental group still retained a low but measurable b-wave magnitude. Scotopic and mixed b-wave data were acquired under dark-adapted conditions to measure rod and maximal rod-cone responses, respectively, while photopic data were acquired under light-adapted conditions to trigger the cone response (FIG. 4B). All conditions exhibited a statistically greater b-wave amplitude in the Tsc1^{-/-} [symbol] group, compared to the control group at all time points. There was a decrease in statistical significance over time that may have been due to the loss of rods early on, leading to diminished differences between the control and experimental ERG recordings at later time points.

D. Robust immunoreactivity of rhodopsin in rods and cone arrestin in the cones of Tsc1^{-/-} [symbol] mice. Rhodopsin (RHO) is a rod-specific protein essential to the phototransduction pathway, and expression levels of this protein are correlated with functional capability phototrausduction. Retinal sections were immunostained for RHO at multiple time points (FIG. 5A), revealing stronger expression in the Tsc1^{-/-} [symbol] mice than in the Tsc1^{loxP/loxP} [symbol] mice at every time point. In both groups, there was a gradual decline in protein expression over time, although by 12 weeks, RHO staining was scarcely observed in the Tsc1^{loxP/loxP} [symbol] mouse while it persisted in the Tsc1^{-/-} [symbol] group. RHO expression was quantified by measuring the width of cells expressing the protein and was found to corroborate the imaging data (FIG. 5B). Sections were also stained for cone arrestin, which is a cone-specific G protein-coupled receptor (GPCR) modulator (FIG. 6A). While Tsc1^{-/-} [symbol] mice showed more robust expression than Tsc1^{loxP/loxP} [symbol] mice at every time point, there was a gradual decline in cone arrestin expression in both groups over time. By 20 weeks, cone arrestin was no longer detectable in the control group, while some indications of its presence were observable in the Tsc1^{-/-} [symbol] mice. Cone density was quantified by counting cell nuclei and was found to be greater in the Tsc1^{-/-} [symbol] group at all time points compared to controls (FIG. 6B).

E. Downstream targets of mTOR were upregulated in [symbol] homozygotes lacking TSC1 . Phosphorylation of the mTOR proteins was assessed by immunofluorescence staining at P21and revealed heightened expression in the experimental group compared to the control group (FIG. 7A). Individual segments of the IS/OS tips were clearly delineated in Tsc1^{-/-} [symbol] when stained for p-mTOR, while they were indistinguishable in the Tsc1^{loxP/loxP} [symbol] controls. A downstream target of this pathway, phospho-S6 (pS6), was also stained and found to be greater in the Tsc1^{-/-} [symbol] mice. Immunoblot revealed a significantly less intense TSC1 band in the knock out mice compared to the controls, which likely arose from Tsc1 expression in cones, bipolar cells, and other retinal cells, as the whole retina was harvested for analysis (FIG. 7B). Conversely, mTOR, phosphorylated mTOR (p-mTOR), and their downstream targets were upregulated in the knock out mice, including (pS6, glucose transporter 1 (GLUT1), and phosphorylated 4E binding protein-1 (p4EBP1), which promotes protein synthesis(24, 25). Conversely, no difference in expression was found in unphosphatated S6 and 4EBP1 in the Tsc^{loxP/loxP} [symbol] versus the Tsc1^{-/-} [symbol] mice. Sterol regulatory element binding protein-1 (SREBP1) is another downstream target that stimulates lipogenesis(26). Phosphorylation of this protein leads to the reduction of lipid synthesis; pSREBP1 was found to be downregulated in Tsc1^{-/-} [symbol] mice, suggesting enhanced lipogenesis. Autophagy protein 5 (Atg5) was also analyzed because inhibiting was shown to cause retinal degeneration in wild type mice ═Zhou, 2015 #441|, although no difference between groups was detected. Changes in protein fold were quantified showed that the greatest difference between groups was in mTOR, p-mTOR, and p4EBP1 levels, all of which were increased in the knock out mice.

F. Safety data for Tsc1^{-/-} [symbol] mice: no tumor formation. Tsc1^{-/-} [symbol] mice were observed for sixteen months (FIG. 8), and then eight organs were dissected for H&E staining. Sections from heart, brain, lungs and other major organs did not show any signs of tumor formation.

### Discussion

Through these data, we demonstrated that both rod and cone degeneration can be rescued in the early stages by ablating the Tsc1 gene specifically in rods. We used morphological assays to determine these results and, to our knowledge, are the first to provide electrophysiology data to demonstrate that restoring balance towards anabolism can be therapeutic. This finding builds off the work of groups like Punzo et al., who found metabolic aberrations in the insulin/mTOR signaling pathway in the cones of four different mouse models of RP. Systemic treatment with insulin encouraged cone survival, suggesting that there may be a metabolic basis to retinal degeneration in these models (20,27). Our paper advances these findings by corroborating their cone results in rods and identifying a potential mechanism to explain how mTOR upregulation promotes anabolism. Notably, we demonstrate that enhancing the mTOR pathway in rods can also promote cone cell survival. This may be due to rod-derived nutritional factors that are required to sustain cones, and thus, promoting rod survival may enhance the release of such factors and concurrently increase preservation of cones (28-30). In a study of red-cone dystrophy, heightened rod-derived cone viability factor (RdCVF) expression led to cone preservation for up to five weeks (31). In comparison, our study achieved cone rescue for up to twenty weeks and rod rescue up to 20 twelve weeks. For therapeutic purposes, either early treatment of rods or simultaneous treatment of rods and cones together may afford the greatest possibility of achieving retinal survival.

Several notable results can be garnered from our data. First, the early stages of degeneration, TSC1 deficiency is effective at retarding degeneration in the ONL (FIG. 2). While both the experimental and control groups experienced decreases in the ONL over time, the rate of degeneration was slower for the experimental group. Surprisingly, the reverse was true in the stages of degeneration, where the rate of degeneration was faster in the experimental group than in the control group. This may indicate that at later time points, rescue is harder to achieve, and perhaps upregulation of mTOR is not sufficient to stave off culminated Ca²⁺/cGMP toxicity caused by the [symbol] mutation (21). An alternative explanation for the slowed photoreceptor dysgenesis could be that TSC1 deficiency in rods improved retinal development as opposed to retarding degeneration. Further studies must be undertaken to distinguish which of these most accurately reflects the physiology of the rescue.

Another notable finding is that the width of the IS/OS layers actually increased during the early stages of degeneration in both groups, although the slope of increase was higher in the Tsc1^{-/-} [symbol] mice than in the controls. Unexpectedly, maintenance of IS/OS genesis occurred despite the ongoing ONL, degeneration. At later time points, however, the IS/OS layer width began to decrease over time, and although the slopes of the groups were comparable, the Tsc^{-/-} [symbol] mouse still maintained a greater width at all time points compared to Tsc1^{-/-} [symbol] mice. These data suggest that perhaps TSC1 deficiency enhances IS/OS biogenesis during early periods of development, or it may slow the rate of IS/OS degeneration. Distinguishing between these hypotheses is beyond the scope of this report, and deeper exploration into this topic should be undertaken.

Our data also demonstrated that TSC1 ablation upregulated the downstream targets of the mTOR pathway, namely pS6,p4EBP, and GLUT1. While the former effectors—pS6 and p4EBP1—stimulate protein synthesis, the lattermost—GLUT1—regulates glucose transport (32). Specifically, GLUT1 increases glucose uptake into cells and its upregulation may be indicative of increased metabolism. Thus, upregulation of anabolic processes such as protein and lipid synthesis in OS may have retarded the cell death signals. Notably, ATG5 expression did not vary between control and experimental mice. Recently, studies showed that deletion of Atg5 in rods and/or cones of wild type mice caused retinal degeneration [Zhou, 2015 #444], although our findings seem to suggest that the deleterious effects may operate through another mechanism. Our data instead align more closely with the theory that upregulation of anabolic processes could be a potential non-gene-specific therapy for patients with retinal degeneration. While enhancing the mTOR pathway could be a potential risk for tumor formation, safety data acquired from knock out mice over sixteen months indicates that TSC1 ablation does not lead to tumor formation, likely because the Cre driver is highly specific to rods and does not target other organs. Our study also shows that early treatment is more effective at slowing degeneration. From our previous studies on the mTOR pathway, we found that similar results can be achieved by silencing Tsc2, and perhaps the combination of knocking out Tsc1 and Tsc2 simultaneously may hold the most potential for abrogating the degeneration that is characteristic of this disease. Additional future studies exploring this strategy would be beneficial to elucidating the mechanistic connection between the upregulated effectors of ananabolism and the slowed neurodegeneration observed.

The genetic heterogeneity of RP poses limitations to monotherapy, i.e. gene-specific treatment. Currently, four gene therapy trials for an early-onset form of RP are ongoing (33-36). Although these trials initially demonstrated some functional improvement, such gene-specific trials are costly. With more than 60 genes implicated in development of RP, this is an impractical strategy that is not amenable to large-scale production or treatment. We have demonstrated here that promoting anabolic pathways in rods can improve their resistance to degeneration in a non-gene-specific manner. Recent research from other groups supports the important role of metabolism, not only in retinal degeneration but also in neural degenerative conditions in general (37-40). Reprogramming a terminally differentiated neuron to remain in an anabolic state is applicable to a diverse range of genetic deficits and could potentially be used to treat multiple neurodegenerative conditions. The mTOR pathway is highly druggable, and appears to be a ubiquitous self-defense mechanism in many types of, cell pathways. Targeting the mTOR pathway is, at the least, a starting point for the development of non-gene-specific therapeutics.

### Materials and Methods

### Animal

The Columbia University Institutional Animal Care and Use Committee (IACUC) approved all experiments prior to study initiation, and mice were used in accordance with the Statement for the Use of Animals in Ophthalmic and Vision Research of the Association for Research in Vision and Ophthalmology and the Policy for the Use of Animals in Neuroscience Research of the Society for Neuroscience.

Three lines of mice were crossed generate the control and experimental mice. [symbol] / [symbol] mice were rederived via oviduct transfer using European Mouse Mutant Archive (EMMA) morulae (21, 41) Pde6g^{CreErO2} mice were generated in the Barbara & Donald Jonas Stem Cell & Regenerative Medicine Laboratory (42-48); and Tsc1^{tm1Djk}/J were purchased from the Jackson Laboratory (Stock No. 005680) (49). All mice were housed in the Columbia University Pathogen-free Eye Institute Annex Animal Care Services Facility and maintained with a 12-h light/12-h light/12-h dark cycle.

[symbol] / [symbol] and [symbol] mice were crossed; their offspring were then bred with Tsc1^{tm1Djk}/J mice. Four generations of backcrosses yielded heterozygous Tsc1^{-/-} [symbol] Pde6g^{CreErT}2 mice (FIG. 1), and six generations of backcrosses yielded homozygous Tsc1^{-/-} [symbol] Pde6g^{CrERT2} mice (all other figures).

At P7, half of the mice from each group injected at P7, 9, and 11 with 100 μg/g body weight of tamoxifen (100 mg/mL in ethanol; catalog T5648Sigma-Aldrich), which was diluted with corn oil to a concentration of 10 mg/mL and mixed thoroughly at 42° C. The rest of the mice were injected in an identical fashion with ethanol (10% in corn oil) instead of tamoxifen. The former served as the experimental group and the latter, the control group. No discrimination based on the sex of the mice was made in regards to group ip assignment.

### Immumunoblotting

Using a previously published method (43, 45), retinae were collected from 3-week-old mice and subsequently homogenized in M-PER Mammalian Protein Extraction Reagent (Prod #78501 Thermo Scientific), which was supplemented with phosphatase inhibitor cocktail 1 (catalog P2850-5ML; Sigma) and protease inhibitor cocktail (catalog P8340-1ML; Sigma). The bicinchonic acid (BCA) protein assay (Thermo Scientific) was used to measure protein concentrations, and sodium dodecyl sulfate-polyacrylamide gel electrophoresis (SDS-PAGE; 4%-15%; Bio-Rad), was used to separate proteins. Samples were then transferred to nitrocellulose (Bio-Rad), blocked in 5% skim milk (902887 MP Biomedicals, LLC), then incubated overnight at 4° C. in the following antibodies; Hamartin/TSC1 Antibody (Cell Signalling Technology, 4906 Anti-mTOR Antibody (Cell Signaling Technology,2972) Anti-mTOR (phospho S2448 antibody (Abcam,ab109268), Anti-S6 Ribosomal Protein (Cell Signaling Technology,2217) Antibody, Phospho-S6 Ribosomal Protein (Ser240/244) (Cell Signaling Technology,5364S) Antibody, Anti-4E- BP1

(Cell Signaling, Technology,5311I1) Antibody, Phosph0-4E-BP1 (Ser65) (174A9) inAb (Cell Signaling Technology,9456), SREBP1 (2A4) (ThormoFisher,MA5-16124) Antibody, Anti-APG5L/ATG5 (Abcam,ab109490) Antibody, Anti-Glucose Transporter GLUT1 (Abcam,40084) Antibody, α-Tubulin Antibody (Cell Signaling Technology,2144S) Antibody, and Anti-beta Actin (Abcam 125248) Antibody. After washing three times in 0.5% PBST (500 μ1 Tween-20 in 1,000 ml PBS), they were then incubated for 1 h at room temperature goat anti-rabbit IgG-HRP secondary antibody (1:2,000, catalog sc-2004, Santa Cruz Biotechnology, Inc.) or rabbit anti-mouse) IgG-HRP sc-358914 HRP conjugated antibody (1:2,000; sc-358914; Santa Cruz Biotechnology Inc.). Chemiluminescent detection (EMD Millipore) using Biomax film (Kodak) revealed membrane proteins. We then measured the level of a particular protein in Tsc1^{-/-} [symbol] and Tsc1^{loxP/loxp} [symbol] mice (n=4 for each group). Image J analysis was used to analyze bands. Protein levels were normalized respective to tubulin levels. Levels from one of the Tsc1^{loxP/loxP} [symbol] mice were normalized to 1, and all other experimental conditions were compared to this. A t-test was used to compare the statistical difference between the two groups.

### ONL Density and Inner/Outer Segment Length Measurement

The cornea and lens were dissected and the vitreous removed from enucleated eyes to isolate the eyecup following mouse euthanasia, performed following established IACUC guidelines and previously described procedures (45) (43). H&E and retinal paraffin sections were prepared at 5 μm in size using Excalibur Pathology, and then they were divided into four regions: peripheral temporal, central temporal; central nasal; and peripheral nasal regions. Photoreceptor nuclei in each region were quantified using Image J (NIH, Bethesda) The ONL thickness was also measured in each of the four quadrants and averaged, the IS/OS length was ascertained in the same manner.

### Immunohistochemistry

Eyes were enucleated and submerged in 4% paraformaldehyde for 1 h at room temperature. Retinae were then dissected from cryoprotected using 30% glucose overnight at 4° C., then sectioned vertically at 10 μm- with a cryostat (Leica). After washing 3 times with phosphate-butlered saline (PBS, pH 7.4), sections were incubated overnight at 4° C. with the following primary antibodies: rabbit anti-cone arrestin (1:5000, Millipore), mouse anti-rhodopsin (1:500, Santa Cruz Biotechnology), Anti-mTOR (Phospho S2448) antibody (Abcam,ab109268), Phospho-S6 Rihosomaal Protein (Ser235/236) Antibody(Cell Signalling Technology,94561 diluted in 5% Chemiblocker (Life Technologies) and 0.3% Triton X-100 in PBS. The sections were subsequently washed in PBS, then incubated with secondary antibodies conjugated to either Alexa 555 or Alexa 488 (1:500, Molecular Probes, Life Teehaologies) for 1 h at room temperature. Sections were washed again with PBS, then for 5 min with 5 μg/ml Hoechst 33342 (Molecular Probes). Sections were analyzed by microscopy (Leica), and only those contain the optic nerve were included for analysis. Five sections were analyzed and averaged for each mouse. The RHO's width was measured at 300 μm from the optic nerve of both sides by Image J(NIH,Bethesda). The average of 5 slices of each mice were analyzed to represent each mouse. Using the same method cone nuclei density was quantified manually.

### ERG

Mice were dark-adapted overnight. Mice were then anesthetized by way of intraperitoneal injection using 1 mL of 100 mg/mL ketamine and 0.1 mL of 20 mg/mL xylazine in 8.9 mL PBS at a concentration of 0.1 mL/10 g BW and placed on heating pads to maintain a 37° C. body temperature. One drop of Tropacamide Ophthalmic Solution (1%, Akorn) was delivered to dilate each, and ten minutes later, electrodes were placed on the conreas and Gonisol of Hypromellose Ophthalmic Demulcent Solution (2.5%, Akron) was administered. ERG recordings were subsequently recorded under dim red light illumination.

Both eyes were simultaneously recorded using the Electrophysiological system (Diagnosys). Pulses of 0.00130 cd/m² and 3 cd/m² (White-6500K) were used to collect rod and maximal rod and cone ERG responses, with 40 to 60 trials per result. To assess the cone response, mice were light-adapted in the Ganzfeld dome for 10 min, then exposed to white flashes while ERGs were recorded. A background of 30 cd/m² (White-6500K) light was used throughout the trial to suppress rod responses. ERGs were recorded at 4, 6, 8, 10, and 12 weeks.

### Statistics

For Tsc1^{-/-} [symbol] and Tsc1^{loxP/loxP} [symbol] mice, to compare differences in mean ERG or histology outcome levels between the two groups, linear mixed models were fit with random intercepts for mouse, since we have two observations per mouse. For the H&E staining histological analysis in the retinal degeneration background, we only have one observation per mouse. To compare histology outcomes between groups at a fixed time point, t-tests were used to compare means. To compare the change in histology outcomes over time between groups, linear regression models were fit, where the predictors were group, time, and a group by time interaction.

For the ERG analysis in the retinal degeneration background, we have two observations per mouse at each time point. To compare ERG outcomes between groups at a fixed time point, linear mixed models with random intercepts for mouse were fit. To compare the trajectory of ERG outcomes over time between groups, linear mixed models with random intercepts for mouse were fit, where the predictors were group, time, time-squared, and interactions between group and time variables. To test whether mean trajectories were different between groups, likelihood ratio tests were used.

We also compared mean differences between groups for the following outcomes: rhodopsin, anti-cone arrestin, and mTOR pathway protein expression levels. For these, we have one observation per mouse, and so, means were compared using t-tests.

### Study Approval

The IACUC of Columbia University approved animal experiments before initiation of the project, and mice were used in accordance with the Statement for the Use of Animals in Ophthalmic and Vision Research of the Association for Research in Vision and Ophthalmology, and, the Policy for the Use of Animals in Neuroscience Research of the Society for Neuroscience.

### References 1. Hartong, D.T., Berson. E. L. and Dryja, T.P. (2006) Retinitis pigmentosa. Lancet, 368, 1795-1809.

2. Hamel, C. (2006) Refunds pigmentosa. Otphanet J. Rare Dis., 1, 40.

3. Fahim. A.T., Daiger, S.P. and Welcher, R.G. (1993) Pagon, R.A., Adam, M.P., Ardiniger, H. H. Wallace, S.E., Amemiya, A., 45 Bean, L.,J.,H., Bird, T.D., Fong, C.T., Mefford, H.C., Smith, R.J. H. and Stephens, K. (eds.), In GeneReviews(R), Seattle (WA), in press.

4. Lin, M.K., Tsai, Y.T. and Tsang, S.H. (2015) Emerging treatments for retinitis pigmentosa: Genes and stem cells, as well as new electronic and medical therapies, are gaining ground. Retin Physician, 12, 51-70.

5. Daiger, S.P., Bowne, S.J. and Sullivan. L.S. (2007) Perspective on genes and mutations causing retinitis pigmentosa. Arch. Ophthalmol, 125, 151-158.

6. Daiger, S.P., Sullivan L.S., Gire, A. I., Birch, D.G., Heckenlively, 55 J.R. and Bowne, S. J. (2008) Mutations in known genes account for 58% of autosomal dominant retinitis pigmentosa (adRP). Adv. Exp. Med.-Biol., 613, 203-209.

7. Sullivan, L.S., Bowie, S.J., Birch, D.G., Hughbanks-Wheaton, D., Heckenlively, J.R., Lewis, R.A., Garcia, C.A., Ruiz, R.S., 60 Blanton, S.H., Northrup, H., et al. (2006) Prevalence of disease-causing mutations in families with autosomal dominant retinitis pigmentosa screen of known genes in 200 families. Invest. Ophthalmol, Vis. Sci., 47, 3052-3064.

8. Friedman, D.S., O'Colmain, B.J., Munoz, B., Tomany, S.C., 65 McCarty, C., de Jong, P.T. Nemesure, B., Mitchell, P., Kempen, J. and Eye Diseases Prevalence Research, G. (2004) Prevalence of age-related macular degeneration in the United States. Arch. Ophthalmol., 122, 564-572.

9. Molday, R.S. and Moritz, O.L. (2015) Photoreceptors at a 70 glance. J. Cell Sci., 128, 4039-4045.

10. Punzo, C., Xiong, W., and Cepko, C.L. (2012) Loss of daylight vision in retinal degeneration: are oxidative stress and metabolic dysregulation to blame? J. Biol. Chem., 287. 1642-1648.

11. Kurihara, T., Westenskow, P.D., Gantner, M.L., Usni, Y., 75 Schultz, A., Bravo, S., Aguilar, E., Wittgrove, C., Friedlander, M., Paris, L.P., et al. (2016) Hypoxia-induced metabolic stress in retinal pigment epithelial cells is sufficient to induce photoreceptor degeneration. Elife, 5, 5, pii: e14319.

12. Veleri, S., Lazar, C.H., Chang, B., Sieving, P.A., Banin, E. and 80 Swaroop, A. (2015) Biology and therapy of inherited retinal degenerative disease: insights from mouse models. Dis, Model Mech., 8, 109-129.

13. Like, D. and Hall, M.N. (2015) mTOR in health and in sick-ness. (Berl), 93, 1061-1073, 85.

14. Martinez de Morentin, P.B., Martinez-Sanchez, N. Roa, J., Ferno, J., Nogueiras, R., Tena-Sempere, M., Dieguez, C. and Lopez, M. (2014) Hypothalamic mTOR: the rookie energy sensor, Curr. Mol. Med., 14. 3-21.

15. Howell, J.J., Ricoult, S.J., Ben-Sahara, I. and Manning, B.D., 90 (2013) A growing role for mTOR in promoting anabolic metabolism. Biochem. Soc. Trans., 41, 906-912.

16. Proud, C.G. (2006) Regulation of protein synthesis by insulin, Biochem. Soc. Trans., 34, 213-216.

17. Astrinidis, A. and Henske, E.P. (2005) Tuberous sclerosis 95 complex: linking growth and energy signaling pathways with human disease. Oncogene, 24, 7475-7481.

18. Xue, H., Ji, V., Wei. S., Yu. Y., Yan, X., Liu, S., Zhang, M., Yao, F., Lan, X. and Chen, L. (2016) HGSD attenuates neuronal ap- optosis through enhancing neuronal autophagy in the brain 100 of diabetic mice: the role of AMP-activated protein kinase. Life Sci., 153:23-34.

19. Evangelisti, C., Cenni, V. and Lattinzi, G. (2016) Potential therapeutic effects of the mTOR inhibitors for preventing ageing and progeria-related disorders, Br. J. Clin. Pharmacol., 105 doi: 10.1111/bcp,12928.

20. Tsang, S.H., Chan. L., Tsai, Y.T., Wu, W.H., Hsu, C.W., Yang, J., Tosi, J., Wert, K.J., Davis, R.J. and Mahajan, V. B. (2014) Silencing of tuberin enhances photoreceptor survival and function in a preclinical model of retinitis pigmentosa (an 110 american ophthalmological society thesis). Trans. Am. Ophthalmol. Soc., 112, 103-115.

21. Punzo, C., Kornacker, K. and Cepko, C.L. (2009) Stimulation of the insulin/mTOR pathway delays cone death in a mouse model ot retinitis pigmentosa, Nat. Neurosci., 12, 44-52, 115

22. Davis, R.J., Tosi, J. Janisch. Kasanuki, J. M., Wang, N.K., Kong, Tsui, I., Cilluffo, M., Woodruff, M.L. Fain, G.L., et al. (2008) Functional rescue of degenerating photoreceptors Malice homozygous for a hypomorphic cGMP phosphodiesterase 6 b allele (Pde6bH620Q). Invest. Ophthalmol. Vis. Sci., 49, 5067-5076.

23. Kurumaji, Y. and Miyazaki, K. (1900) Tiopronin-induced 5 lichenoid eruption in a patient with liver disease and positive patch test reaction to drugs with sulthydryl group. J. Dermatol, 17, 176-181.

24. Olney, J.W. (1968) An electron microscopic study of synapse formation, receptor outer segment development, and other 10 aspects of developing mouse retina. Invest. Ophthalmol., 7, 250-268.

25. Dickinson, J. (1900) Sharing the pain of motherhood. Nurs. Times, 86, 36-38.

26. Gardner, T.W., Abcouwer, S.F., Losiewicz, M.K. and Fort, P.E. 15 (2015) Phosphatase control of 4E-BPI phosphorylation state is central for glycolytic regulation of retinal protein synthesis. Am. J. Physiol Endocrinol Metab., 309, E546-E556.

27. Griffiths, B. Lewis, C.A., Bensaad, K., .Ros, S., Zhang, Q., Ferber, E.C., Konisti, S., Peck, B., Miess, H., East, P., et al. (2013) 20 Sterol regulatory element binding protein-dependent regulation of lipid synthesis supports cell survival and tumor growth. Cancer Metab., 1, 3.

28. Caron, A., Richard, D. and Laplante, M. (2015) The roles of mTOR complexes in lipid metabolism. Annu. Rev., Nutr., 35, 25 321-348.

29. Venkatesh A., Ma, S., Le, Y.Z. Hall, M.N., Ruegg, M.A. and Punzo, C. (2015) Activated mTORC1 promotes long-term cone survival in retinitis pigmentosa mice. J. Clin. Invest., 125, 1446-1458, 30

30. Fridlich, R., Millet-Puel, G., Clerin, E., Delalande, F., Jaillard, C., Blond, F., Perrocheau, L., Reichman, S., Byrne, LC., et al., (2015) Rod-derived cone viability factor promotes cone survival by stimulating aerobic glycolysis. Cell, 161, 817-832.

31. Sahel, J.A., Mohand-Said, S., Leveillard, T., Hicks, D., Picaud, 35 S, and Dreyfus, H. (2001) Rod-cone interdependence: implications for therapy of photoreceptor cell diseases. Prog. Brain Res., 131, 649-661.

32. Mohand-Said, S., Hicks, D., Dreyfus, H. and Sahel, J.A. (2000) Selective transplantation of rods delays cone loss in a retini-40 tis pigmentosa model. Arch. Ophthalmol., 118, 807-811.

33. Byrne, L.C., Dalkara, D., Luna, G. Fisher, S,K., Clerin E., Sahel, J.A., Leveillard, T. and Flannery, J.G. (2015) Viral-mediated RdCVF and RdCVFL expression protects cone and rod photoreceptors in retinal degeneration. J. Clin, Invest., 125, 45 105-116.

34. Zhou, Z., Doggett, T.A., Sene, A., Apte, R.S. and Ferguson, T.A. (2015) Autophagy supports survival and phototrausduction protein levels in rod photoreceptors. Cell Death Differ., 22, 488-498, 50

35. Zhou, Z., Vinberg, F., Schottler, F., Doggett, TA., Kefalov, V.J. and Ferguson, T.A. (2015) Autophagy supports color vision. Autophagy, 11, 1821-1832.

36. Maguire, A.M., High, K.A., Auricchio, A., Wright, S.F. Pierce, E.A., Testa, F., Mingozzi, F., Bennicelli, J.L. Ying, G.S., Rossi, 55 S., et al. (2009) Age-dependent effects of RPE65 gene therapy for Leber's congenital amaurosis: a phase 1 dose-escalation trial. Lancet, 374, 1597-1605.

37. Lopes, V.S. and Williams, D.S. (2015) Gene therapy for the retinal degeneration of usher syndrome caused by muta-60 tions iat MYO07A, Cold Spring Harp, Perspect, Med. 5,

38. Ghazi, N.G. Abboud, E.B., Nowilaty, S.R., Alkuraya, H., Alhommadi, A., Cai, H., Hou, R., Deng W.T. Boye, S.L. Almaghamsi, A., et al. (2016) Treatment of retinitis pigmentosa due to MERTK mutations by ocular subretinal injection of adeno-associated virus gene vector: results of a phase I 65 trial. Hum. Genet., 135, 327-343.

39. Kousal, B., Dudakova, L., filavata, L. and Liskova, P. (2016) Clinical tests testing new therapies for stargardt disease. Cesk. Slov. Oftalmol., 72, 293 -297.

40. Du, J., Rountree, A., Cleghorn, W.M. Contreras, L., Lindsay, 70 K.J., Sadilek, M., Gu, H., Djukovic, D., Raftery, Satrustegui, J., et al., (2016) Phototransduction influences metabolic flux and nucleotide metabolism in mouse retina J. Biol. Chem., 291, 4698-4710.

41. Cepko, C. and Punzo, C. (2015) Cell metabolism: Sugar for 75 sight Nature, 522, 428-429.

42. Demetrius, L.A. and Driver, 3. (2013) Aliheimer's as a metabolic disease. Biogerontology, 14, 641-649.

43. Demetrius, L.A. and Simon, D.K. (2012) An inverse-Warburg effect and the origin of Alzheimer's disease. Biogerontology, 80 13, 583-594.

44. Hart, A.W., McKie, L., Morgan, J.E., Gautier, P., West, K., Jackson, I.J. and Cross, S.H., (2005) Genotype-phenotype correlation of mouse pde6b mutations. Invest Ophthalmol, Vis. Sci., 46, 3443-3450. 85

45. Koch, S.F., Tsai, Y.T., Duong, J.K., Wu, W.H., Hsu, C.W., Wu, W.P., Bonet-Ponce, L., Lin, C.S. and Tsang, S.H. (2015) Halting progressive neurodegeneration in advanced retinitis pigmentosa. J. Clin, Invest., 125, 3704-3713.

46. Tsang, S.H. Gouras, P., Yamashita, C.K., Kjeldbye, H., Fisher, 90 J., Farber, D.B. and Goff, S.P. (1996) Retinal degeneration in mice lacking the gamma subunit of the rod cGMP phosphodiesterase. Science, 272, 1026-1029.

47. Tsang, S.H., Chen, J., Kjeldbye, H., Li, W.S., Simon, M.I., Gouras, P. and Goff, S.P. (1997) Retarding photoreceptor de-95 generation in Pdegtm1/Pdegtm1 mice by an apoptosis suppressor gene. Invest. Ophthalmol. Vis. Sci., 38, 943-950.

48. Tsang, S.H. Burns, M.E., Calvert, P. Gouras P., Baylor, D.A., Goff, S.P. and Arshaysky, V.Y. (1998) Role for the target en-zyme in deactivation of photoreceptor G protein in vivo, 100 Science, 282, 117-121.

49. Tanabe, T. Tsang. Kjeldbye, H. Berns, K., Goff, S. and Gouras, P. (1998) Adeno-associated virus mediated gene transfer into PDE g knockout mouse. Invest. Ophthalmol. Vis. Sci., S5153. in press., 105

50. Tosi, J., Sancho-Pelluz, J., Davis, R.J., Hsu, C.W., Wolpert, K.V., Sengilla, J.D., Lin, C.S.S. and Tsang, S.H. (2011) Lentivirusmediated expression of cDNA and ShRNA slows degeneration in retinitis pigmentosa. Exp. Biol. Med. (Maywood, N.J.), 236, 1211-1217. 110

51. Tsang, S.H., Tsui, I., Chou C. L., Zernant, J., Hsamer, E., Iranmanesh, R., Tosi, J. and Allikmets, R. (2008) A novel mutation and phenotypes in phosphodiesterase 6 deficiency. Am. J. Ophthalmol., 146, 780-788.

52. Kwiatkowski, D.J., Zhang, H., Bandura, J.L. Heiberger, K.M., 115 Glogauer, M., el-Hashemite, N. and Onda, H. (2002) A mouse model of TSC1 reveals sex-dependent lethality from liver hemangiomas, and up-regulation of p70S6 kinase activity in Tsc1 null cells. Hum. Mol. Genet., 11, 525-534.

### Enhancing Glycolysis with Gene Therapy Attenuates Neurodegeneration

### Example 2

A. Generation of experimental and control groups. The third most common cause of autosomal recessive RP is deficiency in the PDE6 enzyme, which controls the depolarization state of rods by regulating cGMP levels (10, 45-47). An established preclinical model for RP involves a homozygous point mutation (H620Q) in the Pde6b gene, which causes delayed-onset RP. Photoreceptor degeneration begins when the mice are two-three weeks old, and by eight weeks of age, nearly all photoreceptors have degenerated and minimal functionality remains (11, 48). For gene therapy in this preclinical mouse model, we generated a tamoxifen-inducible rod photoreceptar-specific Cre recombinase to conditionally inactivate expression of the loxP-modified Sirt6^{tm1.1Cxd} gene. Experimental mice were bred from three homozygous strains: [symbol] , Pde6g^{CreERT2}, and Sirt6 mice. Thus, the experimental mice were homozygous for [symbol] and Sirt6^{tm1.1Cxd}, and heterozygous for Pde6g^{CreERT2}. Half of the experimental offspring were injected with tamoxifen and served as the experimental group, henceforth referred to as Sirt6 deficient (Sirt6^{-/-} [symbol] Pde6g^{CreERT2}) and denoted, Sirt6^{-/-} [symbol] , whereas the other half were injected with oil and served as controls (Sirt6^{loxP/loxP} [symbol] Pde6g^{CreERT2}), denoted Sirt6^{loxP/loxP} [symbol] , Upon tamoxifen induction, the Pfrh6g^{CreERT2} recombinase excised exons 2 and 3 of the Sirt6 gene in the retinae of Sirt6^{-/-} [symbol] mice. A 524-bp band in DNA amplified from the outer nuclear layer (ONL) of retinae collected from tamoxifen-treated mice, but not in DNA amplified from the ONL of control mice. A 2,048-bp nonrecombined product was also noted in other organs of both Sirt6^{-/-} [symbol] and Sirt6^{loxP/loxP} [symbol] control mice, including the liver lungs, kidneys, and pancreas. This demonstrates the specificity of the Pde6g^{CreERT2} recombinase for the retina and suggests that off-target effects from the tamoxifen injection were limited.

B. Functional rescue of retinal ellectrophysiological properties in Sirt6^{-/-} [symbol] mice. To assess the extent retinal function rescue, we performed electroretinography (ERG) analysis at weekly intervals (FIG. 9). The trend lines projected for the Sirt6^{-/-} [symbol] mice compared to control Sirt6^{loxP/loxP} [symbol] mice show a statistically significant difference in scotopic, photopic, and mixed ERG responses. Under dark-adapted conditions, the mixed and scotopic b-wave responses measuring the electrophysiological function of rod cells (FIG. 9A top and bottom were significantly higher in Sirt6^{-/-} [symbol] mice compared with Sirt6^{loxP/loxP} [symbol] mice. Light-adapted conditions were used to measure the cone response and produced higher b-wave responses (FIG. 9A, middle) in the Sirt6^{-/-} [symbol] mice compared with controls. At 20 weeks, the scotopic, photopic, and mixed b-wave responses were undetectable in the Sirt6^{loxP/loxP} [symbol] controls, whereas the Sirt6^{-/-} [symbol] mice retained measurable functionality. We found that the greatest difference between the b-wave responses of the treated and control groups occurred at 4 weeks in rod cells under scotopic conditions FIG. 9B, bottom). This difference diminished over time, no statistically significant difference by 8 weeks. A similar pattern was observed for the mixed rod-cone response (FIG. 9B, top), although a statistical difference between the groups remained even at 8 weeks. Notably, for the cone response, the b-wave amplitude showed no statistically significant difference between Sirt6^{-/-} [symbol] and Sirt6^{loxP/loxP} [symbol] mice at 4 weeks, but became statistically significant at the and 6- 8-week time points (FIG. 9B, middle), as would be expected in RP. In general, rods are the primary targets of RP-related degeneration, whereas cones are unaffected until later in the disease, after most of the rods have died. Consistent with the later onset of cone dysfunction in RP, a measureable difference in the ERG data acquired from cones in Sirt6^{-/-} [symbol] and control Sirt6^{loxP/loxP} [symbol] appeared later (FIG. 9B, middle), when the rods had deteriorated. These data suggest that Sirt6 knockout preserves the electrophysiological function of rods and cones and slows the rate of functional loss.

C. Morphological rescue of retina layers in Sirt6^{-/-} [symbol] mice. To observe retinal morphology at 3, 4, 6, and 8 weeks, retinae from experimental and control mice were harvested. Some were stained with hematoxylin and eosin (H & E) (FIG. 10A), while others were observed under transmission electron microscopy (TEM) FIGS. 10B-C). For the histology sections, the thickness of the IS/OS layers and the number of nuclei in the ONL were quantified (FIGS. 10D-E). The IS/OS of Sirt6^{-/-} [symbol] mice were more robust in thickness and the ONL number of nuclei was denser compared with Sirt6^{loxP/loxP} [symbol] mice at all but the last time point (FIGS. 10A, D-E). Specifically, both the IS/OS layer and ONL were viable until week 6 in the treated group, whereas they were indiscernible by that time in the untreated group. However, at eight weeks, there was no statistical difference remaining, and only a few nuclei in the ONL were detected in each group. Furthermore, TEM (FIGS. 10B-C) allowed for a minute inspection of the mitochondria and OS for comparison between groups. It was found that the treated group had significantly healthier mitochondria, as indicated by greater cristae folds as well as longer OS, with more concentrated disc numbers. While these differences decreased over time, the Sirt^{-/-} [symbol] mice still appeared to have overall mitochondria and OS morphologies, even at 8 weeks.

D. Sirt6 deficiency promotes photoreceptor survival and preserves cellular OS. The OS layer is composed of both rod and cone outer segments. To distinguish between each cell type, retinal sections obtained at 3, 4, and 6 weeks were immunostained with antibodies directed against rhodopsin, to identify rods (red), and opsin, to identify short-wavelength cones (blue) (FIGS. 11A-D). We found a thicker width of both rods' and cones' OS in the Sirt6-deficient mice compared with the control group at every time point. Cone cells were still detectable at week 6 in the Sirt6^{-/-} [symbol] mice, whereas only a few rods and cones with abnormal morphology: remained in the Sirt6^{loxP/loxP} [symbol] mice OS layer by that time. Additionally, the OS in the Sirt6^{-/-} [symbol] mice were longer than those in the control mice at every time point. This suggests not only that photorecoptor death is slowed in the treated mice, but also that the cell carphology is able to resist deteriation for a longer time. Although the gene responsible RP in the Pde6b model is expressed exclusively in rods, cone cell death charteterizes the late stages of the disease and causes blindness. Anti-cone arrestin staining (green) identified cone cells 2-(4-amidinophenyl)-1H-indole-6-carboxamidine (DAPI) staining (blue) identified retinal nuclei (FIGS. 11E-F). Cone cell density was higher at every time point in treated (Sirt6^{-/-} [symbol] ) compared with untreated mice (FIG. 11F, P<0.001). In untreated mice, almost no cones could be detected at 12 weeks, whereas a thin layer of cones remained visible in the Sirt6^{-/-} [symbol] mice. In both groups, the greatest decrease in cone cell density occurred between 4 and 6 weeks, which is congruent with the rate of degeneration typical of this mouse model.

E. Sirt6-deficiency in wild type background produces no phenotypic changes in functionality or morphology of photoreceptors. Sirt6^{-/-} [symbol] mice were compared to Sirt6^{loxP/loxP} [symbol] at 3 months following tamoxifen injection. ERG recordings of mixed, scotopic, and photopic b-waves were obtained (FIGS. 12A-C). No statistical difference between the groups was observed for any of the light adaptation conditions. Retinal sections to from each group were harvested at 4 months post tamoxifen injection and were subjected to H&E staining. No difference in OS and ONL thickness were observed (FIGS. 12D-F). These results suggest that the Sirt6 deficiency has neither a beneficial nor deleterious effect in the wild type background.

F. Gene therapy-mediated functional and morphological photoreceptor rescue in a preclinical RP model. To translate tamoxifen-induced Sirt6 ablation into the format of a potential interventional trial, we employed a Sirt6_shRNA somatic gene therapy strategy. An AAV2/8 Sirt6_shRNA vector was transduced into the dorsal retina of the right eye in [symbol] mice (FIG. 13A). The dorsal retina of the left eye was injected with phosphate buffered saline (PBS), whereas the ventral side of all eyes was left untreated. The mixed, photopic, and scotopic ERG b-wave values were recorded and averaged at 4 weeks of age (FIGS. 13B-D). The eyes injected with the Sirt6_shRNA vector showed significantly higher b-wave values. H & E staining of retinal sections from each eye four weeks post injection revealed a measurable increase in the photoreceptor density of vector-injected eyes compared with PBS-injected or untreated eyes (FIGS. 13E-H). Notably, even within the same eye, only the dorsal side of right eyes showed increased photoreceptor density, whereas the untreated ventral side of the same eye showed degeneration.

G. Up-regulation of proteins involved in glycolysis in Birth-deficient retinae. Because of the critical role of Sirt/6 in regulating metabolism, we expected there to be a metabolic basis to the observed photoreceptor rescue (FIGS. 9-13). Thus, we assessed changes in the flux of the downstream targets of Sirt6 through glycolysis and anabolic pathways. Immunoblots retinal extracts from Sirt6-deficient retinae were collected, and levels of the proteins involved in glycolysis were measured by immunoblotting (FIG. 14). These included HIF1a and HIF2a, glucose transporters GLUt1, GLUT2, and MYC, all of which were increased compared with the levels observed in extracts from control retinae (FIG. 14). Up-regulation of these downstream factors suggests increased levels of anabolism in Sirt6^{-/-} [symbol] retinae. Phosphorylated lactate dehydrogenase A (p-LDHA) and von Hippel-Lindau (VHL) protein were down-regulated in the knockout mice, while LDHA levels showed no difference between groups. LDHA is an enzyme that converts pyruvate into lactate, and some studies have suggested that phosphorylation of LDHA increases its activity and induces the Warburg effect (49, 50), although this does not appear to be the case in the Sirt6-deficient retina. VHL regulates protein degradation and down-regulates HIF2A, which is reflected in our data by the decreased levels of VHL and the increased levels of HIF2A.

H. Analysis of downstream metabolites upregulated by Sirt6 deficiency. To directly determine the effects of Sirt6 deficiency on the rate of glycolysis and the TCA cycle, we used gas chromatography-mass spectrometry (GC-MS) analysis to determine flux through key metabolic intermediates (FIG. 15A) at the onset of degeneration, as measured by fractional abundance of each intermediate. Forty-five minutes after intraperitoneal (IP) injection of ¹³C-labeled glucose, retinae were harvested and the distribution of metabolite isotope isomers isotopomers) was analyzed to measure the enrichment of each intermediate by determining the ratio of labeled ions to total ion intensity at 3 and 4 weeks of age (FIG. 15B). ¹³C-labeled glucose is transformed into pyruvate and lactate through glycolysis, and the pyruvate enters into the mitochondria and is incorporated into the TCA cycle as metabolic intermediates and amino acids. Retinae in Sirt6^{-/-} [symbol] mice showed much higher ¹³C-incorporated intermediates in both glycolysis and the TCA cycle as compared with controls at both time points. However, at four weeks the statistical difference of the metabolites was diminished, and some even lost statistical significance. This suggests that the retinal degeneration may have advanced beyond what Sirt6 deficiency could compensate for. The relative abundance of metabolic intermediates in the control and experimental mice, as measured by the total number of carbons labeled in each metabolite, was significantly increased for glycolytic intermediates more so than for TCA cycle intermediates, indicating that Sirt6 deficiency, while increasing overall metabolism, exerts its greatest effects on glycolysis (FIG. 15C-D). As shown in FIG. 15D, this shift may be beneficial for enhancing anabolic processes in the Sirt6^{-/-} [symbol] retinae. To determine whether the absolute abundance of the metabolites was also increased, we performed liquid chromatography-mass spectrometry (LC-MS) at three weeks of age to analyze intermediates involved in the major glucose metabolism pathways, detecting 126 in total. More than 100 of the metabolites were upregulated in the Sirt6-deficient mice compared with controls, although the difference was statistically significant only for some of them. All metabolites with a statistically significant difference (P<0.05) are shown (FIG. 15E) and were upregulated in the Sirt6^{-/-} [symbol] mice compared with controls at three weeks. These experiments were repeated at four weeks, and the majority of the metabolites were still increased in the Sirt6deficient retinae, although the statistical difference diminished between weeks three and four. Of the eleven metabolites identified at week three as having the most statistically significant difference between the experimental and control groups, only one maintained significance at week four: oxidize glutathione. Overall, these results indicate an upregulation of glycolytic metabolism in the Sirt6-deficient retina, althouch this trend decreases over time.

I. PFK shRNA viral knockdown exacerbates retinal degeneration in [symbol] mice, Phosphofructokinase (PFK) is a key regulator of glycolysis and serves as the rate-limitinci glycolytic enzyme in metabolism (51). This enzyme has 1 isoforms (52), including PFK−M, −L, and −P. To isolate the effect of glycolysis on retinal degeneration from the effects of Sirt6 deficiency, a lentivirus expressing shRNA for one of the three isoforms was injected into [symbol] mice. Additionally, a lentivirus carrying shRNA for all three isoforms was injected into a fourth group of mice. ERG mixed, scotopic, and photopic b-wave values acquired at 4 weeks post injection revealed a statistically significant deerease in scotopic b-wave amplitudes in the mice injected with the tripartite shRNA combination of PFK−M+L+P (FIGS. 16A-C). A considerable but non-statistically significant decrease was observed in the mixed and photopic b-wave values. H & E-stained retinal sections confirmed these findings (FIGS. 16D-F). A statistically significant decrease in ONL and OS layer thicknesses was detected in the PFK−M+L+P shRNA group. As PFK is directly responsible for activating glycolysis, these findings, part particularly the histological results, support our hypothesis that a metabolic imbalance may underlie or, at the least, exacerbate retinal degeneration.

### Discussion

Photorecptor degenerative conditions are pervasive, affecting over 9 million Americans, and are devastating, often leading to loss of the ability to conduct activities of daily living (1, 2). RP, one of the most devastating retinal degenerative disorders, is associated with at least 64 genes encoding mostly rod cell-specific proteins that lead to cell death when improperly formed (2, 53). There is currently no cure available, and although gene therapy interventions recently reached clinical trials, the heterogeneity of gene deficits that cause RP is a fundamental limitation of these studies (54, 55), because the strategy involves a monotherapy, which cannot be used to treat RP caused by mutations in more than one gene. Thus, a non-gene-specific rescue strategy (12, 13) of increase biosynthetic fuel and survival by reprogramming of glycolysis is highly desirable.

Normally, photoreceptors are among the most metabolically active cells in the body (16-20), converting 80-96% of incoming glucose into lactic acid via aerabic glycolysis (56); (21). To function properly, rods regenerate the phagocytosed OS layer daily. RP-induced rod cell death arises from an inability to perform this protein and lipid renewal process (2, 23), and the IS/OS biogenesis is suspected to be insufficient. We therefore hypothesized that degeneration can be rescued by tipping the cell's metabolic balance toward anabolism through inhibition of Sirt6, a transcriptional repressor of glycolytic flux. This is based on the theory that aerobic respiration typically leads to catabolic processes, while anaerobic metabolism caters to anabolism (57, 58).

SIRT6 is known as a regulator of glycolysis in the context of cancer (39), but it has yet to be studied as a therapeutic target for reprogramming metabolism in neurodegeneration. Studies show that SIRT6 causes glucose to be preferentially shuttled into aerobic respiration, and in its absence, anaerobic metabolic pathways, glycolysis in particular, are prioritized instead. Here, we ablated Sirt6 in a preclinical model of RP to assess the role metabolism plays in retinal degeneration. We thus hypothesized that cells could be preserved by upregulating anabolism, which could be achieved by prioritization of glycolysis caused by Sirt6 deficiency.

In the present study, electrophysiological and a anatomic rescue of the [symbol] preclinical model were achieved with both germline and AAV2/8 Sirt6_shRNA somatic gene therapy. ERG recordings from Sirt6^{-/-} [symbol] mice showed higher b-wave amplitudes, thicker OS/ONL, and greater rod and cone nuclear density compared with controls. Because the [symbol] mutation is hypomorphic, the PDE6 protein remains partially active, providing an explanation for the measurable ERG recordings obtained in the control mice before degeneration overwhelmed the functionality of the photoreceptors (11). Loss of Sirt6 increased levels of HIF1A, HIF2A. GLUT1, GLUT2, and MYC, which are key drivers of biosynthesis. Increased levels of HIF1A, GLUT1 have previously been associated with photoreceptor survival (59);(12). In one study, injection of insulin in four unique RP mouse models was shown to delay degeneration of cones, and increased levels of GLUT1 and HIF1a were detected (12). In another study involving tuberous sclerosis complex 2 (TSC2) ablation, the mTOR pathway was enhanced, leading to upregulation of GLUT1, which was hypothesized to protect cones from degeneration(13). Additionally, GLUT1 has been shown to play an important role in protecting rods via the activity of rod-derived cone viability factor (60) deficiency allowed maxmimal activity of enzymes involved in both glutaminolysis, driven by MYC (61), and enhanced retinal glycolytic flux. Increasing levels of HIF1A and MYC have been shown to up-regulate LDHA in cancer cells (50), which was not observed in the Sirt6-deficient retina. The discrepancy between our data and our expectations on the basis of prior studies could be explained by a negative feedback mechanism in which Sirt6 knockout increases anabolic and glycolytic metabolites, including lactate (FIG. 15), and the resultant high levels of lactate turn off LDHA activity and phosphorylation. Furthermore, the ¹³C-labeled isotopomer data reliably confirmed that glycolytic flux was preferentially up-regulated (62, 63).

Overall, the metabolome shifted to accumulate biosynthetic intermediates in the OS of rods lacking Sirt6, including intermediates in multiple metabolic pathways including glycolysis, the TCA cycle, and glutaminol, (FIGS. 7A-D). These changes are consistent with up-regulated anabolism, which, in turn, would be expected to improve retinal function and counter cell death. Additionally, the expression of the metabolites was enhanced in Sirt6 [symbol] mice, as assessed by LC-MS). After statistical analysis, we confirmed that glycolytic metabolism was enhanced as indicated by increased flux from glucose into pyruvate and lactate. The intermediates of the TCA cycle were also upregulated, but to a lesser extent (FIGS. 15C-D). For example, the flux from glucose to aKG was dramatically increased in the Sirt6-deficicient mice compared to controls. Plausibly, Sirt6-deficiency enhanced glycolysis, and the glycolysis-derived pyruvate entered the mitochondria to increase [symbol] KG and other intermediates. Overall, the fold chancy its lactate and pyruvate, both endpoints of glycolysis, was significantly higher compared to metabolites of the TCA cycle, suggesting a preference for glycolytic metabolism in the Sirt6-deficient mice.

To confirm that glucose metabolism influences retinal degeneration, we knocked down all PFK isoforms in the [symbol] background, as this enzyme regulates the rate-limiting step in glycolysis, and its intermediates feed into the TCA cycle, Knockdown significantly decreased ERG scotopic b-wave amplitudes and cell layer thickness (FIG. 16), suggesting that glycolytic aberrations do a#affect retinal degeneration. However, there are multiple downstream enzymes and alternative pathways that could be responsible for these results, and further experiments be needed to explore other possibilities.

Despite the reduced rate of disease progression, a limitation of the approach described here is that Sirt6 ablation counteracted, but could not completely halt, cell death. The primary cell death drivers, Ca²⁺ and cGMP toxicity arising from PDE6 dysfunction, were not reversed by Sirt6 inhibition. Continued cone cell death may be due to loss of rod-derived cone viability after loss of rods (64-68) or because or a reduction in the levels of the antioxidant transcription factor, NRF2(69, 70). Why Sirt6 deficiency confers early-stage but not long-term disease amelioration remains unclear. Two hypotheses have been put forth: the first suggests that Sirt6 deficiency causes metabolic changes that slow retinal degeneration, while the second suggests that the metabolic changes lead to enhancement of photoreceptor biosynthesis during mouse retinal develeopment. Achieving long-term efficacy is a universal limitation for most gene therapy interventions. In recent clinical trials of retinal gene therapy in Leber's Congenital Amaurosis, some research subjects showed functional improvements (26-33). However, several follow-up studies clearly showed that photoreceptor loss had not been halted, or even slowed (54, 71-73). Another limitation of our data is that it may only be applicable to RP associated with the Pde6 mutation. Future studies should be undertaken to explore whether metabolism plays a similar role in other RP disease origins.

Sirt6_gene therapy achieved similar efficiency as the Opsin::Pde6b gene therapy, where a viral-transgene expressing wild type Pde6b was able to induce partial functional and localized morphological rescue of photoreceptors (11). A future strategy would be to apply a bipartite gene therapy vector to simultaneously treat a patient's specific mutation(s) while also reprogramming anabolism. The “one-two punch” that would be provided by this combination therapy could potentially prevent future damage. Another alterative may be to combine down-regulation of Sirt6 with up-regulation of the mTORC1 pathway, as described by Venkatesh et al. (13). This would involve inhibition of Tsc1 in the cones of Sirt6^{-/-} [symbol] mice. Up-regulation of anabolism in rods combined with simultaneous up-regulation of mTORC1 in cones could, perhaps, have additive effects on photoreceptor survival compared with the use of each strategy in isolation. Perhaps such a strategy would not merely slow retinal degeneration, but terminate it. Targeting other key regulators to reprogram metabolism may also prove fruitful in developing treatments for RP. In general, the metabolic underpinning of RP is not understood, although mouse models have been used to explore the role metabolism plays in degeneration. In our previous study, we showed that the Rd1mouse, which harbors a Pde6 mutation, had decreased metabolites compared to wild type mice (74). In this study, we used the [symbol] mouse to determine the effects of metabolism in retinal degeneration.

The present results also support recent evidence whic showed that up-regulation of glycolysis in cone photoreceptors drives their survival and is crucial to photoreceptor health (60). They also align with reports that increased levels of HIF1a, HIF2a, GLUT1, GLUT2. MYC, and the other glycolytic enzymes are associated with photoreceptor survival (59). Together, the data and results described herein provide a foundation that supports a role for reprogramming metabolism to treat neurodegenerations. Neurodegenerative conditions such as Alzheimer's disease. Parkinson's disease, and glaucoma have also been suggested to arise from metabolic aberrations (54, 75, 76). As photoreceptors are, at their most basic level, specialized neurons, the present strategy illustrated for reprogramming metabolism by targeting Sirt6 signaling may thus be translatable to halting other degenerative disorders of the central nervous system as well (77, 78).

### Testing SIRT6 Inhibitors in a PDR (Diabetic Retinopathy Disease) Model

Additionally, it is noted that the present strategy: illustrated for reprogramming metabolism by targeting Sirt6 signaling will be applicable to methods using any SIRT6 inhibitors, including, but not limited to small molecules or mimetics based on any of the following: of fenugreek seed extract, Vitexin (isolated from Hawthorn tree berries), quercetin, naringenin, vitexin. SYN17739303, BAS13555470, SYN 10366754, and BAS00417531. Another example of a SIRT6 inhibitor is Vitexin (isolated from Hawthorn tree berries), which in certain instances can be given by PO or formulated in a sustained-release form, biodegradable implant in the human vitreous. Additionally, any suitable mode of delivery can be utilized for administering one or more of the ^{SIRT}6 inhibitors. Additional exemplary SIRT6 inhibitors have been identified and discussed by Yasuda et al. (Anal Chem. 2011 Oct 1;83(19):7400-7), Schlicker et al. (Aging, 2011 September; 3(9): 852-872), Singh et al. (J Chromatogr B Analyt Technol Biomed Life Sci. 2014 Oct 1; 0; 105-111), and Parenti at al. (J Med Chem 2014 Jun 12;57(11)4796-804). These and additional exemplary SIRT6 inhibitors are expected to be useful alone, or in combination in aspects of the present invention.

A diabetic retinopathy murine model, described in Wert et al. Signal Transduction and Targeted Therapy (2016) 1:16005; will be utilized to test the following SIRT6 inhibitors: including, but not limited to small molecules or mimetics based on any of the following: of fenugreek seed extract. Vitexin (isolated from Hawthorn tree berries), quercetin, naringenin, vitexin, SYN17739303, BAS13555470, SYN10366754, and BAS00417531. These Chx 10-cre;Vhl^{flox/flox} mice exhibit features including vasculature defects which make it useful as a preclinical model for diabetic retinopathy and ischemic retinopathies. It is expected that one or more of the following features will be analyzed upon administration of each SIRT6 inhibitor: vitreous hemorrhage, neovascularization, intraocular pressure, cataract formation, anterior synechia, neovascular glaucoma. It is anticipated that ocular treatment with the tested SIRT6 inhibitors will result in improvement in one or more of the above features, and may also include improvements such as slowing retinal degeneration, and/or neovascularization and improving conditions such as diabetic retinopathy and/or ischemic retinopathies.

### Materials and Methods

Animals

The Columbia University Institutional Animal Care and Use Committee (IACUC) approved all experiments prior to initiation. Mice were used in accordance with the Statement for the Use of Animals in Ophthalmic and Vision Research of the Association for Research in Vision and Ophthalmology and the Policy for the Use of Animals in Neuroscience Research of the Society for Neuroscience.

Three lines of mice were crossed to develop the breeding strains, Sirt6^{tm1.1Cxd}/J mice (79) were purchased from the Jackson Laboratory; [symbol] mice were rederived via oviduct transfer using European Mouse Mutant Archive (EMMA) morulae 11, 80); and Pde6g^{CreERT2} mice were generated in the Barbara & Donald Jonas Stem Cell & Regenerative Medicine Laboratory (5, 6, 81-86). All mice were housed in the Columbia University Pathogen-free Eye Institute Annex Animal Care Services Facility and maintained with a 12-h light/12-h dark cycle.

[symbol] mice were crossed with Pde6g^{CreERT2} mice, and their offspring were bred with Sirt6^{tm1.1Cxd/J} mice. Six generations of backcrosses were required to generate breeding mice. The resulting progeny were homozygous for all alleles of interest (Pde6b, Sirt6, and Pde6g), but some were wild type at Pde6g, whereas others possessed the Pde6g^{CreERT}2 mutation. We isolated these two lines for use as breeding strains. Crossing the breeding strains produced the experimental mice, which are homozygous at the Pde6b and Sirt6 loci, and heterozygous at the Pde6g locus.

At P7, half of the experimental mice were given a 100 μg/g body weight (BW) injection of tamoxifen (100 mg/ml in ethanol; catalog T5648; Sigma-Aldrich), which was diluted with corn oil to a concentration of 10 mg/ml and thoroughly mixed at 42° C. One injection was administered on P7, P8, and P9. The other half of the experimental mice were injected with ethanol (10% in corn oil) following the same dosage as tamoxifen and served as the control group. There was no discrimination based on the sex of the mice.

### Genotyping

Recombination of the Sirt6 allele was accomplished through tamoxifen-induced Sirt6 LoxP removal at P7. To verify Sirt6 recombination in rods, 10-μm frozen sections of the retina collected, and DNA was extracted from the ONL using a 30-gauge needle and surgical microscope (m690; Leica). PCR was completed as previously outlined (81). All other organs were subsequently collected. Three primers were used to target the Sirt6 DNA sequence: forward 5′ GCTAATGGGAACGACGAGACCAA 3′ (SEQ ID NO:3); internal 5′ ACCCACCTCTCTCCCCTAAA 3′ (SEQ ID NO:4); and reverse 5′ GCGTCCACTTCTCTTTCCTG 3′(SEQ ID NO:5). The recombination allele of Sirt6 was amplified using the forward and reverse primers to produce the 524-bp fragment.

DNA for genotyping was extracted from mice tails. Sirt6 mice genotypes were confirmed using the forward and internal primers. This primer set amplifies 390-bp fragments for wild type Sirt6 mice and 444-bp fragments for mice with conditional alleles that contain the LoxP insertion site in intron 1. Genotyping [symbol] required the following primers: forward 5′ TGCCACGACATCGACCACCCG3′ (SEQ ID NO:6) and reverse 5′GCCATCCCTGCCTTCCCTTGG3′ (SEQ ID NO:7). This set amplifies a 598-bp fragment, which was sequenced to confirm the presence of the H620Q point mutation. Pde6g^{CrdERT2} mice require the following primers: forward 5′ GGTCAGATTCCAGTGTGTGGG 3′ (SEQ ID NO:8) and reverse GTTTAGCTGCTGGCCCAAATGTTG 3′ (SEQ ID NO:9) ID NO:9). This primer set produces 514-bp fragments for wild type mice and 715-bp fragments for mice with conditional alleles.

### Immunoblotting

Retinae were harvested from 3-week-old mice, homogenized in M-PER Mammalian Protein Extraction Reagent (Prod #78501 Thermo Scientific) supplemented with phosphatase inhibitor cocktail 1 (catalog P2850-5ML; Sigma) and protease inhibitor cocktail (catalog P8340-IML; Sigma), using a previously published method (5, 6), and protein concentrations were measured using the bicinchoninic acid (BCA) protein assay (Thermo Scientific). Sodium dodecyl sulfate-polyacrylamide gel electrophoresis (SDS-PAGE; 4%—15% Bio-Rad) was used to separate proteins, which were subsquently transferred to nitrocellulose (Bio-Rad). After blocking in 5% skim milk (903887 MP Biomedicals, LLC), membranes were incubated overnight at 4° C. in the 0 antibodies: rabbit polyclonal anti-Sirt6 -ChIP Grade (1:500; catalog ab105391.Abcam) rabbit polyclonal anti-von Hippel-Lindau-N-terminal (anti-VHL; catalog ab135576; Abcam); mouse monoclonal anti-glucose transporter GLUT1 (1;2000; catalogue ab40084; Abcam); mouse monoclonal anti-glucose transporter GLUT2 (1:2000; catalog ab104622; Abcam); mouse monoclonal HIF-1α(1:500; catalog MBS143031; Novas Biologicals); rabbit polyclonal HIF-2 α/EPAS1antibody (1:1,000; catalog NB100122; Novus Biologicals); mouse monoclonal [9E10]to c-Myc (HRP) (1:1,000: catalog ab62928; Ahcam); rabbit polyclonal LDHA antibody (1:500; #2012; Cell Signaling); phospho-LDHA (Tyr10; 1:1,000; catalog 8176S; Cell Signaling Technology); and mouse anti-β-actin (1:1,000; catalog ab125248; Abcam). They were then washed three times in 0.5% PBST (500 μl Tween-20 in 1,000 ml PBS) and incubated for 1 h at room temperature in goat anti-rabbit IgG-HRP secondary antibody (1:2,000; catalog sc-2004; Santa Cruz Biotechnology, Inc.) or rabbit anti-mouse IgG-HRP sc-358914 HRP conjugated antibody (1:2,000; sc-358914; Santa Cruz Biotechnology Inc.). Membrane proteins were revealed by chemiluminescent detection (EMD Millipore) using Biomax film (Kodak).

### ONL Density and Inner/Outer Segment Length Measurement

Mice were euthanized and eyes enucleated according to established IACUC guidelines following previously described procedures (5, 6), The cornea and lens were dissected and the ous removed, isolating the eyecup. Excalibur Pathology prepared H & E and retinal paraffin sections (5 μm). To quantify cell numbers and thickness, each section was divided into four regions: peripheral temporal; central temporal; central nasal; and peripheral nasal quadrants. ONL density was measured by counting the number of photoreceptor nuclei in each quadrant. This value was then divided by the length of the ONL measured in the four quadrants and the thickness of the section. The IS/OS length was determined by measuring the average thickness of the IS/OS layer in the four quadrants using Image J.

### Transmission Electron Microscopy

Retinae were sectioned, fixed in half-strength Karnovsky fixative, stained with uranyl acetate and lead citrate, embedded in Spurrs medium, cut at 90 nm, collected on grids, and examined by transmission electron microscopy using a Zeiss 190. Images were digitzied and viewed in Adobe Photoshop, and slight adjustments were made to the brightness to distinguish mitochondria and outer segment layers more clearly.

### Immunohistochemistry

For frozen sections, eyes were enucleated and placed in 4% paraformaldehyde for 1 h at room temperature. After fixation, retinae were dissected from the eyecup cryoprotected in 30% glucose overnight at 4° C. and sectioned vertically at 10 μm with a cryostat (Leica). Sections evere washed 3 times with phosphate-buffered saline (PBS, pH 7.4) and incubated overnight at 4° C. with the following primary antibodies: rabbit anti-cone arrestin (1:5000, Millipore), mouse anti-rhodopsin )1:500, Santa Cruz Biotechnology), and rabbit anti-blue opsin (1:200, Millipore) diluted in 5% Chemiblocker (Life Technologies) and 0.3% Triton X-100 in PBS. After washing in PBS, the sections were incubated with secondary antibodies conjugated to either Alexa 555 (1:500, Molecular Probes, Life Technologies) for 1 h at room temperature. Sections were then washed with PBS and incubated for 5 min with 5 μg/ml Hoechst 33342 (Molecular Probes) and analyzed by confocal microscopy (Nikon A1). Only sections containing the optic nerve were included for analysis. Cone nuclei density was quantified manually on each section. Five sections through the optic nerve were collected and averaged for each mouse.

### ERG

After mice were dark-adapted overnight, recordings were obtained under dim red light illumination. Mice were anesthetized with an anesthetic solution (1 mg/ml ketamine and 0.1 mL 20 mg/mL xylazine in 8.9 ml PBS) at a concentration of 0.1 mL/10 g BW injected in the intraperitoneal region. Heating pads were used to maintain body temperature at 37° C. One drop of Tropicamide Ophthalmic Solution (1%-Akorn) was administered in each eye for dilation. Ten minutes later, electrodes were placed on the corneas and Goniosol Hypromellose Ophthalmic Demulcent Solution (2.5%, Akron) was applied.

Both eyes were recorded simultaneously. Electrophysiological system (Diagnosys) was used to record ERG responses concurrently from both eyes. For rod and maximal rod and cone ERG responses, pulses of 0.00130 cd/m² and 3 cd/m² (White-6500K) were used. Each result represents the average of 40 to 60 trials. For cone responses, mice were light-adapted in the Ganzfeld dome for 10 min. A background of 30 cd/m² (White-6500K) was present throughout the trials to suppress rod function. ERGS were recorded using white flashes. ERGs were recorded at 4-8 and 10 weeks.

### Subretinal AAV Injections

The AAV2/8(Y733F)-Sirt6_shrNA virus (FIG. 13A) was injected into the subretinal area at P4 in [symbol] mice. The eyelids of one eye of the newborn mouse were opened artificially using microsurgery scissors. AAV was transduced into the dorsal retinae by subretinal injection. Dorsal of the right eyes were injected with AAV2/8(Y733F)-Siri6_shRNA vector, whereas the left eyes were injected with same dose or PBS. The ventral side of all eyes was left untreated. Mice were examined by direct ophthalmoscopy to rule out retinal detachment at P21. ERG and histology data were collected at 4 weeks of age.

Three isoforms of PFK shRNA lentiviral particles were purchased from Sigma-Aldrich (ID number: GFP: MFCD07785395, PFK−M:TRCN0000319795, PFK−L.:TRCN0000360434, PFK−P:TRCN0000274701). Lentivirus was injected into the subretinal area at P4 -P6 in [symbol] mice. The same subretinal injection method was used as in the Sirt6 AAV injection, described above. ERG data were collected at 3 weeks of age, while histology samples were collected at 4 weeks of age at 0.3 mm distal from the optic nerve head.

### ¹³C-Labeled Isotope Tracing and Mass Spectrometry

¹³C-labeled D-Glucose (U-¹³C₆, 99%) was purchased from Cambridge Isotope Laboratory, Inc. and was injected intraperitoneally (500 mg/Kg) in four groups: Sirt6^{loxP/loxP} [symbol] and Sirt6^{-/-} [symbol] mice. After 45 minutes retinae were promptly isolated from the mice, rinsed in PBS, and flash-frozen in liquid nitrogen. The retinae were subsequently homogenized a mixture of methanol, chloroform, and water (700:200;50). The metabolites were dried, derivatized, and analyzed by GC-MS (Agilent 7890/5975C) as previously reported (87, 88). The chromatograms were analyzed using Agilent Chemstation software. The measured distribution of mass isotopologues was corrected for based on the natural abundance of isotopes using the software IsoCor. The fractional abundance of labeled ions to total ion intensity was determined. Data was collected for Sirt6^{loxP/loxP} [symbol] and Sirt6^{-/-} [symbol] mice at P21 and P28.

Steady state metabolites were measured by LC-MS as previously reported by Du et al. 2015 (87). Three-week old control and experimental mice were sacrificed and retinae were collected, rinsed in PBS, and flails frozen in liquid nitrogen. Retinae were harvested at each time point and metabolites extracted in cold 80% methanol and quantified by Agilent 1260 LC Agilent Technologies, Santa Clara, CA)-AB Sciex QTrap 5500 mass spectrometer (AB Sciex, Toronto, ON, Canada) system.

### Statistics

All data were analyzed using Excel, State 12.1, and R 3.1.1. Mice were divided into two groups: control (Sirt6^{loxP/loxP} [symbol] ) and Sirt6-deficient (Sirt6^{-/-} [symbol] ). ERG outcomes were measured over time for between-group comparison at specific time points (e.g., at 4 weeks). For analyses comparing groups at a fixed time point, linear mixed models with random intercepts were fit to the data because each mouse contributes two data points (one per eye). ERG measurements were used as outcome measurements and the assigned group was the predictor.

The trajectory of ERG outcomes was also compared between groups using data from all time points. Linear mixed models were fit as before, but the predictors in this model were group, time, time squared, and interaction terms for group, three and group, and time squared. The quadratic component was included after graphically examining the individual mouse trajectories. This model was compared to a model without interaction terms using a likelihood ratio test to determine whether the groups had different trajectories.

For analyses of IS/OS thickness and ONL nuclei density H & E sections, and for rhodopsin OS width and S-cone opsin OS width, sections from mouse retinae were taken at each time point, and multiple images of each section were collected and averaged. Each mouse only contributes one observation, so we can consider the observations as independent. Two-tailed t tests were used to compare controls to Sirt6-deficient mice, and P values<0.05 were considered significant.

Image J analysis software was used to analyze bands in Western Blots. Protein levels were normalized respective to Actin levels. Levels from one of the Sirt6^{oxP/loxP} [symbol] mice were normalized to 1, and all other experimental conditions were compared to this. A t-test was used to compare the statistical difference between the two groups.

Ten C¹³-labeled metabolites were measured to ascertain the ratio of labeled metabolite abundance to innate abundance, and these values were plotted. We also compared the abundance of glucose metabolites by liquid chromatography mass-spectrometry. The levels of abundance were log10-transformed and normalized to actin levels. Levels of the most statistically significantly changed metabolites involved in the glycolysis pathway were measured and plotted based on abundance. To compare groups for the previously mentioned outcomes, we used a two-sample t test. To compare injection of AAV2/8(Y733F) Sirt6_(shRNA vector vs. PBS, and the PFK virus injection vs. GFP injection, we used a paired design, where the right eye of a mouse was injected with vector and the left with PBS. Then mixed ERG b-wave values were recorded and compared using a paired t test.

### Study Approval

The IACUC of Columbia University approved all experiments prior to study start. Use of mice wasaccordance with the Statement for the Use of Animals in Ophthalmic and a Research of the Association for Research in Vision and Ophthalmology and the Policy for the Use of Animals in Neuroscience Research of the Society for Neuroscience.

### References

1. Hamel C. Retinitis pigmentosa. Orphanet journal of rare diseases. 2006; 1(40.
2. Hartong DT, Berson EL, and Dryja TP. Retinitis ligmentosa. Lancet 2006; 368 (9549):1795-809,
3. Shen B, Zhang W, Zhang J. Zhou J, Wang J, Chen L, Wang L, Hodgkins A, lyer V, Huang X, et tal. Efficient genome modification by CRISPR-Cas9 nickase with minimal off-target effects, Nature methods. 2014; 11(4)399-402.
4.Wu, WH, Tsai YT, Justus S. Lee T, Zhang L, Lin CS, Bassuk AG, Mahajan VB, and Tsang SH. CRISPR repair reveals causative mutation in a preclinical model of retinitis pigmentosa. Mol Ther. 2016.
5. Tsang SH, Gouras P, Burns ME, Calvert PD, Gouras P, Baylor DA, Goff SP, and Arshavsky VY. Role for the target enzyme in deactivation of photoreceptor G protein in vivo. Science. 1998; 282(5386):117-21
6. Tsang SH, Gouras P, Yamashita CK. Kjeldbye H, Fisher J, Farber DB, and Goff SP, Retinal degeneration in mice lacking the gamma subunit of the rod cGOMP phosphodiesterase. Science. 1996; 272(5264):1026-9.
7. Wert RJ, Davis Sancho-Pelluz J, Nishina PM, and Tsang SH, Gene therapy provides long-term visual function in a pre-clinical model of retinitis pigmentosa. Human molecular genetics. 2013; 22(3):558-67.
8. Aherne A, Kennan A, Kenna PF, McNally N, Lloyd DG, Alberrs IL, Kiang AS, Humphries MM, Ayuso C, Engel PC, et al. On the molecular pathology of neurodegeneration in IMPDH1-based retinitis pigmentosa. Human molecular genetics. 2004:13(6):641-50.
9. Wert KJ, Lin JH, and Tsang SH. General pathophysiology in retinal degeneration. Developments in ophthamology. 2014; 53(33-43.
10. Dryja TP, Rucinski DE, Chen SH, and Berson EL. Frequency of mutations in the gene encoding the alpha subunit of rod cGMP-phosphodiesterase in autosomal recessive retinitis pigmentosa. Investigative ophthalmology & visual science. 1999:40(8)1859-65.
11. Davis RJ, Tosi J, Janisch KM, Kasanuki JM, Wang NK, Kong J, Tsui I, Cilluffo M, Woodruff ML, Fain GL, et al. Functional rescue of degenerating photoreceptors in mice homozygous for a hypomorphic cGMP phosphodiesterase 6 b allele (Pde6bH620Q). Investigative ophthalmology visual science. 2008; 49(11):5067-76.
12. Punzo C, Komacker K, and Cepko CL. Stimulation of the insulin/mTOR pathway delays cone death in a mouse model of retinitis pigmentosa. Nature neuroscience. 2009; 12(1.):44-52.
13. Venkatesh A. Ma S, Le YZ Hall MN, Ruegg MA, and Punzo C. Activated mTORC1 promotes long-term cone survival in retinitis pigmentosa mice. The Journal of clinical investigation. 2015; 125(41:1446-58.
14. Lehninger AL, Nelson DL, and Cox MM. Lehninger principles of biochemistry. New York: W.H. Freeman; 2013.
15. Lin MK, Tsai Y-TT, and Tsang SH. Emerging Treatments for Retinitis Pigmentosa: Genes and stem cells, as well as new electronic and medical therapies, are gaining ground. Retinal physician. 2015; 12(52-70.
16. Okawa H, Sampath AP, Laughlin SB, and Fain GL ATP consumption by mammalian rod photoreceptors in darkness and in light. Current biology: 2008; 18(24):1917-21.
17. Nakanishi S. Second-order neurones and receptor mechanisms in visual- and olfactory- information processing. Trends in neurosciences. 1995; 18(8)359-64.
18. Linsenmeier RA. Effects of light and darkness on oxygen distribution and consumption in the cat retina. The Journal of general physiology. 1986; 88(4):521-42.
19. Ahmed J, Braun RD, Dunn R, Jr., and Linsemneier RA. Oxygen distribution in the macaque retina. Investigative ophthalmology & visual science. 1993; 34(3):516-21.
20. Birol G, Wang S, Budzynski E, Wangsa-Wirawan ND, and Linsenmeier RA. Oxygen distribution and consumption in the macaque retina. American journal of physiology Heart and circulatory physiology. 2007; 293(3):H1696-704.
21. Du J, Rountree A, Cleghorn WM, Contreras L, Lindsay KJ, Sadilek M, Gu H, Djukovic D, Raftery D, Satrustegui J, et al. Phototransduction influences metabolic flux and nucleotide metabolism in mouse retina. The journal of biological chemistry. 2015.
22. Fariss RN, Li ZY, and Milam AH. Abnormalities in rod photoreceptors, amacrine cells, and horizontal cells in human retinas with retinitis pigmentosa, American journal of ophthamology. 2000; 129(2)215-23.
23. Curico CA, Sloan KR, Kalina RE, and Hendrickson AE. Human photoreceptor topography. The Journal of comparative neurology. 1990; 292(4):497-523.
24. Maguire AM, Simonelli F, Pierce EA, Pugh EN, Jr., Mingozzi F, Bennicelli J, Banfi S, Marshall KA, Testa F, Surace EM, et al, Safety and efficacy of gene transfer for Leber's congenital amaurosis. The New England journal of medicine, 2008; 358(21):2240-8.
25. Ashtari M, Zhang H, Cook PA, Cyckowski LL, Shindler KS, Marshall KA, Aravand P, Vossough A, Gee JC, Maguire AM, et al. Plasticity of the human visual system after retinal gene therapy in patients with Leber's congenital amaurosis. Science translational medicine. 2015; 7(296):296ra110.
26. Bainbridge JW, Smith M. Barker SS, Robbie S. Henderson R, Balaggan K, Viswanathan A, Holder GE, Stockman A, Tyler N, et al, Effect of gene therapy on visual function in Leber's congenital amaurosis. The New England journal of medicine. 2008:358(21):2231-9.
27. CideCiyan AV, Hauswirth WW, Aleman TS, Kaushal S, Schwartz SB, Boye SL, Windsor EA, Conlon TJ, Sumaroka A, Pang JJ, et al. Human RPE65 gene therapy for Leber congenital amaurosis: persistence of early visual improvements and safety at 1 year. Human gene therapy. 2009; 20(9):999- 1004.
28. Maguire AM, High KA, Auricchio A, Wright JF, Pierce EA, Testa F., Mingozzi F. Bennicelli JL, Ying GS, Rossi S, et al. Age-dependent effects of RPE65 gene therapy for Leber's congenital amaurosis: a phase 1 dose-escalation trial. Lancet. 2009:374(9701):1597-605.
29. Simonelli F, Maguire AM, Testa F, Pierce EA, Mingozzi F, Bennicelli JL, Rossi S. Marshall K, Banfi S. Surace EM, et al., Gene therapy for Leber's congenital amaurosis is safe and effective through 1.5 years after vector administration. Molecular therapy: the journal of the American Society of Gene Therapy. 2010; 18(3) 643-50.
30. Jacobson SG, Cideciyan AV, Ratnakararm R, Heon E, Schwartz SB, Roman AJ, Peden MC, Aleman TS, Boye SL, Sumaroka A, et al. Gene therapy for leber congenital amaurosis cased by RPE65 mutations: safety and efficacy in 15 children and adults followed up to 3 years. Archives of ophthalmology. 2012; 130(1):9-24.
31. Bennett J, Ashtari M, Wellman J, Marshall KA, Cyckowski LL, Chung DC, McCague S, Pierce EA, Chen Y, Bennicelli JL, et al. AAV2 gene therapy readministration in three adults with congenital blindness. Science translational medicine. 2012; 4(120):120ra15.
32. Ashtari M, Cyckowski LL, Monroe JF, Marshall KA, Chung DC, Auricchio A, Simonelli F, Leroy BP, Maguire AM, Shindler KS, et al, The human visual cortex responds to gene therapy-mediated recovery of retinal function. The Journal of clinical investigation. 2011; 121(6):2160-8.
33. Testa F, Maguire AM, Rossi S. Pierce EA, Melillo P, Marshall K, Banfi S, Surace EM, Sun J, Acerra C, et al. Three-year follow-up after unilateral subretinal delivery of adeno-associated virus in patients with Leber congenital Amaurosis type 2. Ophthalmology, 2013:120(6):1283-91.
34. Amado D, Mingozzi F, Hui D, Bennicelli JL, Wei Z, Chen Y, Bote E, Grant RL, Golden JA, Narfstrom K, et al. Safety and efficacy of subretinal readministration of a viral vector in large animals to treat congenital blindnoss. Science translational medicine. 2010; 2(21):21ra16.
35. Vandenberghe LH, Bell P, Maguire AM, Cearley CN, Xiao R, Calcedo R, Wang L, Castle MJ, Maguire AC, Grant R, et al. Dosage thresholds for AAV2 and AAV8 photoreceptor gene therapy in monkey. Science translational medicine. 2011:3(88):88ra54.
36. Daiger SP. Bowne SJ, and Sullivan LS. Perspective on genes and mutations causing retinitis pigmentosa. Archives of ophthalmology. 2007; 125(2):151-8.
37. Lin M, Kim SH, Zhang L, Tsai Y, and Tsang SH. Rod metabolic demand drives progression in retinopathies. Taiwan Journal of Ophthalmology. 2015:5(3):105-8.
38. Yau. KW. Phototransduction mechanism in retinal rods and cones. The Friedenwald Lecture. Investigative ophthalmology & visual science. 1994; 35(I): 9-32.
39. Martinez-Pastor B, and Mostoslaysky R. Sirtuins, metabolism, and cancer. Front Pharmacol. 2012; 3(22).
40. Zhong L. D'Urso A, Toiber D, Sebastian C, Henry RE Vadysirisack DD, Guimaraes A. Marinelli B, Wikstrom JD, Nir T, et, al. The histone deacetylase Sirt6 regulates glucose homeostasis via Hif1alpha. Cell. 2010; 140(2); 280-93.

Zwaans BM, and Lombard DB. Interplay bet een sirtuins, MYC and hypoxia-inducible factor in cancer-associated metabolic reprogramming. Dis Model Mech. 2014; 7(9):1023-32.
42. Kleszcz R, Paluszczak J, and Baer-Dubowska W. Targeting aberrant cancer metabolism—The role of sirtuins. Pharmacological reports: PR. 2015; 67(6): 1068-80.
43. Etchegaray JP, Zhong L, and Mostoslavsky R. The histone deacetylase SIRT6: at the crossroads between epigenetics, metabolism and disease. Current topics in medicinal chemistry. 2013; 13(23):2991, -3000.
44. Silberman DM, Ross K, Sande PH, Kubota S, Ramaswamy S, Apte RS, and Mostoslavsky R. SIRT6 is required for normal retinal function, PloS one. 2014; 9(6): e98831.
45. Hahn LB, Berson EL, and Dryja TP. Evaluation of the gene encoding the gamma subunit of rod phosphodiesterase retinitis pigmentos. Investigative ophthalmology & visual science. 1994; 35(3):1077-82.
46. Huang SH, Pittler SJ, Huang X, Oliveira Berson EL, and Dryja. TP Autosomal recessive retinitis pigmentosa caused by mutations in the alpha subunit of rod cGMP phosphodiesterase. Nature genetics. 1995; 11(4):468-71.
48. Danciger M, Blaney J, Gao YQ, Zhao DY, Heckenliyely JR, Jacobson SG, and Farber DB. Mutations in the PDE6B gene autosomal recessive retinitis pigmentosa. Genomics. 1995; 30(1):1-7.
48. Tsang SH, Chan L, Tsai YT, Wu WH, Hsu CW, Yang J, Tosi J, Wert KJ, Davis RJ and Mahajan VB. Silencing of tuberin enhances photoreceptor survival and function in a preclinical model of retinitis pigmentosa (an american ophthalmological society thesis). Transactions of the American Ophthalmological Society. 2014; 112(103-15.
49. Valvona CJ, Fillmore HL, Nunn PB, and Pilkington GJ. The Regulation and Function of Lactate Dehydrogenase A: Therapeutic Potential in Brain Tumor. Brain pathology. 2016; 26(1):3-17.
50. Fan J, Hitosugi T. Chung TW, Xie J, Ge Q, Gu Polakiewicz RD, Chen GZ, Boggon TJ, Lonial S, et al. Tyrosine phospharylation of lactate dehydrogenase A is important for NADH/NAD(+) redox homeostasis in cancer cells. Molecular and cellular biology. 2011:,31(24):4938-50.
51. Uyeda K. Phosphofructokinase. Adv Enzymol Relat Areas Mol Biol. 1979; 48(193-244.
52. Dimauro S, Akman O, and Hays AP. Disorders of carbohydrate metabolism. Handb Clin Neurol, 2007; 86(167-82.
53. Diager SP, Sullivan LS, Gire AI, Birch DG, Heckenlively JR, and Bowne SJ. Mutations in known genes account for 58% of autosomal dominant retinitis pigmentosa (adRP). Advances in experimental medicine and biology. 2008; 613(203-9.
54. Bainbridge JW, Mehat MS Sundaram V, Robbie SJ, Barker SE, Ripamonti C, Georgiadis A, Mowat FM, Beattie SG, Gardner PJ, et al. Long-term effect of gene therapy on Leber's congenital amaurosis. The New England journal of medicine. 2015; 372(20):1887-97.
55. Solonis MA, del Pozo-Rodriguez A, Apaolaza PS, and Rodriguez-Gascon A. Treatment of ocular disorders by gene therapy. Eur J Pharm Biopharm. 2015; 95(Pt B): 331-42.
56. Hurley JB, Lindsay KJ, and Du J. Glucose, lactate, and shuttling of metabolites in vertebrate retinas. Journal of neuroscience research. 2015; 93(7):1079-92.
57. Vander Heiden MG, Cantley LC, and Thompson CB. Understanding the Warburg effect: the metabolic requirements of cell proliferation. Science. 2009; 324(5930):1029-33.
58. Vazquez A, Liu J, Zhou Y, and Oltvai N. Catabolic efficiency of aerobic glycolysis: the Warburg effect revisited. BMC Syst Biol. 2010,4(58.
59. Chen B, and Cepko CL. HDAC4 regulates neuronal survival in normal and diseased retinas, Science. 2009; 323(5911):256-9.
60. Ait-Ali N, Fridlich R, Millet-Puel G, Clerin E, Delalande F, Jaillard C, Blond F, Perrocheau L, Reichman S, Byrne LC, et al. Rod-derived cone viability factor promotes cone survival by stimulating aerobic glycolysis. Cell. 2015,161(4):817-32.
61. Wise DR, DeBerardinis RJ, Mancuso A, Sayed N, Zhang XY, Pfeiffer HK, Nissim I., Daikhin E., Yudkoff M, McMahon SB, et al. Myc regulates a transcriptional program that stimulates mitochondrial glutaminolysis and leads to glutamine addiction. Proceedings of the National Academy of Sciences of the United States of America, 2008; 105(48); 18782-7.
62. Maher EA, Marin-Valencia Bachoo RM, Mashimo T, Raisanen J, Hatanpaa KJ, Jindal Jeffrey FM, Choi C, Madden C, et al. Metabolism of [U-13 C]glucose in human brain tumors in vivo. NMR in biomedicine. 2012; 25(11):1234-44.
3. Buescher JM, Antoniewicz MR, Boros LG, Burgess SC, Bnunengraber H, Clish CB, DeBerardinis RJ, Feron O, Frezza C, Ghesquiere B, et al. A roadmap for interpreting (13)C metabolite labeling patterns from cells, Current opinion in biotechnology. 2015; 34(189-201.
64. Yang Y. Moband-Said S, Danan A, Simmonutti M, Fontaine V, Clerin E, Picaud S. Leveillard T, and Sahel JA. Functional cone rescue by RdCVF protein in a dominant model of retinitis pigmentosa. Mol Ther. 2009; 17(5):787-95.
65. Leveillard T, and Sahel JA Rod-derived cone viability factor for treating diseases; from clinic to redox signaling. Science translational medicine. 2010; 2(26):26ps16.
66. Leveillard T, Mohand-Said S. Lorentz O, Hicks D, Fintz AC, Clerin E, Simonutti M, Forster V, Cavusoglu N, Chalmel F, et al. Identification and characterization of rod-derived cone viability factor; Nature genetics. 2004; 36(7):755-9.
67. Mohand-Said S, Deudon-Combe A, Hicks D, Simonutti M, Forster V, Fintz AC, Leveillard T, Dreyfus H. and Sahel JA. Normal retina releases a diffusible factor stimulating cone survival in the retinal degeneration mouse. Proceedings of the National Academy of Sciences of the United States of America. 1998; 95(14):8357-62.
68. Mohand-Said S, Hicks D, Dreyfus H, and Sahel JA. Selective transplantation of rods delays cone loss in a retinitis pigmentoso model. Archives of ophthalmology, 2000; 118(6):807-11.
69. Xiong W, MacColl Garfinkel AE, Li Benowitz LI, and Cepko CL. NRF2 promotes neuronal survival in neurodegeneration and acute nerve damage. The Journal of clinical investigation. 2015,125(4):1433-45.
70. Cepko C, and Punzo C. Cell metabolism: Sugar for sight. Nature. 2015; 522(7557):428-9.
71. Cideciyan AV, Jacobson SG, Beltran WA, Sumaroka A, Swider M, Iwabe S, Roman AJ, Olivares MB, Schwartz SB, Komaromy AM, et al. Human retinal gene therapy for Leber congenital amaurosis shows advancing retinal degeneration despite enduring visual improvement. Proceedings of the National Academy of Sciences of the United States of America. 2013; 110(6):E517-25.
72. Jacobson SG, Cideciyan AV, Roman AJ, Sumaroka A, Schwartz SB, Heon E, and Hauswirth WW. Improvement and decline in vision with gene therapy in childhood blindness. The New England journal of medicine. 2015; 372(20):1920-6.
73. Wright AF. Long-term effects of retinal gene therapy in childhood blindness. The New England journal of medicine. 2015; 372(20): 1954-5.
74. Du J, Rountree A. Cleghorn WM, Contreras L, Lindsay KJ. Sadilek M, Gu H, Djukovic D, Raftery D. Satrustegui J, et al. Phototransduction Influences Metabolic Flux and Nucleotide Metabolism in Mouse Retina. The Journal of biological chemistry. 2016:291(9):4698-710.
75. Demetrius LA, Magistretti PJ, and Pellerin L. Alzheimer's disease: the amyloid hypothesis and the Inverse Warburg effect. Front Physiol. 2014:5(522. Demetrius LA. and Simon DK. An inverse-Warburg effect and the origin of Alzheimer's disease, Biogerontology. 2012; 13(6):583-94.
77. Mariani AP. Neuronal and synaptic organization of the outer plexiform layer of the pigeon retina. The American journal of anatomy. 1987; 179(1):25-39.
78. Masland RH. The neuronal organization of the retina. Neuron. 2012;76(2):266-80.
79. Kim HS, Xiao C, Wang RH, Lahusen T, Xu X, Vassilopoulos A, Vazquez-Ortiz G, Jeong WI, Park O, Ki SH, et al. Hepatic-specific disruption of SIRT6 in mice results in fatty liver formation due to enhanced glycolysis and triglyceride synthesis. Cell metabolism. 2010,12(3):224-36.
80. Hart AW, McKie L, Morgan JE, Gautier P, West K, Jackson IJ, and Cross SH. Genotype-phenotype correlation of mouse pde6b mutations. Investigative ophthalmology & visual science. 2005; 46(9):3443-50.
81. Koch SF, Tsai YT, Duong JK, Wu WH, Hsu CW, Wu WP, Bonet-Ponce L, Lin CS, and Tsang SH. Halting progressive neurodegeneration in advanced retinitis pigmentosa. The Journal of clinical investigation. 2015; 125(9):37O4-13.
82. Tsang SH, Chen J, Kjeldbye H, Li WS, Simon MI, Gouras P, and Goff SP. Retarding photoreceptor degeneration in Pdegtm1/Pdegtm1 mice by an apoptosis suppressor gene. Investigative ophthalmology & visual science. 1997; 38(5):943-50.
83. Tanabe T. Tsang SH, Kjeldbye H, Berns K, Goff S, and Gouras P. Adeno-associated virus mediated gene transfer into pde g knockout mouse. Investigative ophthalmology & visual science. 1998:S5153.
84. Tosi J, Davis RJ, Wang NK, Naumann M, Lin CS, and Tsang SH, shRNA knockdown of guanylate cyclase 2e or cyclic nucleotide gated channel alpha 1 increases photoreceptor survival in a cGMP phosphodiesterase mouse model of retinitis pigmentosa. J Cell Mol Med. 2011; 15(8): 1778-87.
85. Tsang SH, Tsui I, Chou CL, Zemant J, Haamer E, Iranmanesh R, Tosi J, and Allikmets R. A novel mutation and phenotypes in phosphodiesterase 6 deficiency. American Journal of ophthalmology. 2008; 146(5):780-8.
86. Tsang S, Woodruff M, Lin C, Jacobson B. Naumann M, Hsu C, Davis R, Cilluffo MC, Chen J, and Fain G. Effect of the ILE86TER mutation in the γ subunit of cGMP phosphodiesterase (PDE6) on rod photoreceptor signalling. Cellular Signalling. 2012; 0(0.
87. Du J, Linton JD, and Hurley JB. Probing Metabolism in the Intact Retina Using Stable Isotope Tracers. Methods in enzymology. 2015; 561(149-70.
88. Du J, Cleghorn W. Contreras L, Linton JD. Chan GC, Chertov AO. Saheki T, Govindaraju V, Sadilek M, Satrustegui J, et al. Cytosolic reducing power preserves glutamate in retina. Proceedings of the National Academy of Sciences of the United States of America. 2013; 110(46):18501-6.

Many modifications and variations of this invention can be made without departing from its spirit and scope, as will be apparent to those skilled in the an. The invention is defined by the terms of the appended claims, along with the full scope of equivalents to which such claims are entitled. The specific embodiments described herein, including the following examples, are offered by way of example only, and do not by their details limit the scope of the invention.

All references cited herein are incorporated by reference to the same extent as if each individual publication, database entry (e.g. Genbank sequences or GeneID entries), patent application, or patent, was specifically and individually indicated to be incorporated by reference. This statement of incorporation by reference is intended by Applicants, pursuant to 37 CFR, § 1.57(b)(1), to relate to each and every individual publication, database entry (e.g. Genbank sequences or GeneID entries), patent application, or patent, each of which is clearly identified in compliance with 37 C.F.R. § 1.57(b)(2), even if such citation is not immediately adjacent to a dedicated statement of incorporation by reference. The inclusion of dedicated statements of incorporation by reference, if any, within the specification does not in any way weaken this general statement of incorporation by reference. Citation of the references herein is not intended as an admission that the reference is pertinent prior art, nor does it constitute any admission as to the contents or date of these publications or documents.

The present invention is not to be limited in scope by the specific embodiments described herein. Indeed, various modifications of the invention in addition to those described herein will become apparent to those skilled in the art from the foregoing description and the accompanying figures. Such modifications are intended to fall within the scope of the appended claims.

The foregoing written specification is considered to be sufficient to enable one skilled in the art to practice the invention. Various modifications of the invention in addition to those shown and described herein will become apparent to those skilled in the art from the thregoing description and fall within the scope of the appended claims.

What is claimed is:

1. A method of increasing glycolysis in a neuronal cell comprising decreasing a level and/or activity of TSC1, SIRT6, or a combination thereof, in the neuronal cell.

2. The method of claim 1, wherein the neuronal cell is a cone cell or a rod cell, or combination of cone cells, rod cells, and/or other retinal cells.

3. The method of claim 1, wherein increasing glycolysis by decreasing a level and/or activity of TSC1, SIRT6, or a combination thereof comprises administering an effective amount of an inhibitor selected from the group consisting of proteins, nucleic acids, chemicals and combinations thereof.

4. The method of claim 3, wherein the nucleic acid is selected from the group consisting of antisense oligonucleotide, siRNA, shRNA, gRNA and combinations thereof.

5. The method of claim 1, wherein the decreasing, comprises administering an effective amount of an inhibitor of TSC1, SIRT6 or a combination thereof.

6. A method of increasing neuronal survival patient(s) in need thereof; comprising altering glycolysis by decreasing a level and/or activity of TSC1, SIRT6, or a combination thereof, in the neuronal cell.

7. The method of claim 6, wherein the neuronal cell is a cone cell, a rod cell, or a retinal cell, or a combination of cone cells, rod cells, and/or retinal cells.

8. The method of claim 6, wherein the decreasing comprises administering an effective amount of an inhibitor of TSC1, SIRT6, or a combination thereof.

9. The method of claim 8, wherein the inhibitor is selected from the group consisting of proteins, nucleic acids, and combinations thereof.

10. The method of claim 9, wherein the nucleic acid is selected from the group consisting of antisense oligonucleotide, siRNA, shRNA, gRNA, and combinations thereof.

11. The method of claim 6, wherein the patient is suffering from one or more retinal degenerative diseases selected from the group consisting of retinitis pigmentosa (RP), age-related macular degeneration (AMD), or glaucoma, or one or more neurodegenerative diseases including Alzheimer's Parkinson's, Huntington's, Amyonotrophic lateral sclerosis (ALS), Lewy body dementia, and combinations thereof.

12. A method of increasing photoreceptor survival comprising altering glycolysis by decreasing a level and/or activity of TSC1, SIRT6, or a combination thereof, in a photoreceptor cell.

13. The method of claim 12, wherein the photoreceptor cell is a cone cell, a rod cell, or a retinal cell, or a combination of cone cells, rod cells, and/or retinal cells.

14. The method of claim 12, wherein the decreasing comprises administering an effective amount of an inhibitor of TSC1, SIRT6, or a combination thereof.

15. The method of claim 14, wherein the inhibitor is selected from the group consisting of proteins, nucleic acids, chemicals, and combinations thereof.

16. The method of claim 15, wherein the nucleic acid is selected from the group consisting of antisense oligonucleotide, siRNA, shRNA, gRNA, and combinations thereof.

17. A method of increasing photoreceptor survival in a patient in need thereof, comprising administering to the patient a therapeutically effective amount of; a recombinant adeno-associated viral (AAV) vector encoding an inhibitor of Tsc1, Sirt6, or other metabolic reprogramming agent, or an inhibitor or activator of anabolism.

18. The method of claim 17, wherein the recombinant AAV vector is an AAV2 vector.

19. The method of claim 17, wherein the AAV vector is an AAV8 vector.

20. The method of claim 17, wherein the AAV vector is administered by intravitreal injection.

21. The method of claim 17, wherein the AAV vector is administered by subretinal injection.

22. A method of increasing photoreceptor survival in a patient in need thereof, comprising administering to the patient a therapeutically effective amount of:
(a) a first recombinant adeno-associated viral (AAV) vector, wherein the first recombinant AAV vector comprises,(i) a first sequence(s) encoding at least one guide RNA that hybridizes to endogenous Tsc1 and/or Sirt6 gene in the patient, and,
(b) a second recombinant AAV vector comprising a nucleic acid sequence encoding a Cas nuclease; wherein the Cas nuclease cleaves the endogenous Tsc1 or Sirt6 gene creating a Tsc1 and/or Sirt6 knockout of the endogenous Tsc1 and or Sirt6 gene in the patient.

23. The method of claim 22, wherein the first AAV vector and/or the second AAV vector are an AAV2 vector.

24. The method of claim 22, wherein the first AAV vector and/or the second AAV vector are an AAV8 vector.

25. The method of claim 22, wherein the Cas nuclease is Cas9.

26. The method of claim 22, wherein the first AAV vector and/or the second AAV vector are administered by intravitreal injection.

27. The method of claim 22, wherein the first AAV vector and/or the second AAV vector are administered by subretinal injection.

28. A method of increasing neuronal survival in a patient in need thereof, comprising administering a therapeutically effective amount of:
a recombinant adeno-associated viral (AAV) vector encoding an inhibitor of Tsc1 , Sirt6, or other metabolic reprogramming agent, or an inhibitor or activator of anabolism, to at least one neuron in the patient.

29. The method of claim 28, wherein the AAV vector is an AAV2 vector.

30. The method of claim 28. wherein the AAV vector is an AAV8 vector.

31. The method of claim 28, wherein the AAV vector is administered by intravitreal injection.

32. The method of claim 28, wherein the AAV vector is administered by subretinal injection.

33. A method of increasing neuronal survival in a patient in need thereof, comprising administering to the patient a therapeutically effective amount of
(a) a first recombinant adeno-associated viral (AAV) vector, wherein the first recombinant AAV comprises, (i) a first sequence(s) encoding at least one guide RNA that hybridizes to endogenous Tsc1 and/or Sirt6 gene in the patient, and,
(b) a second recombinant AAV viral vector comprising a nucleic acid sequence encoding a Cas nuclease; wherein the Cas nuclease cleaves the endogenous Tsc1 and/or Sirt6 gene creating a Tsc1 and/or Sirt 6 knockout of the endogenous Tsc1 or Sirt6 gene in the patient.

34. The method of claim 33. wherein the first AAV vector and/or the second AAV vector are an AAV2 vector.

35. The method of claim 33, wherein the first AAV vector and/or the second AAV vector are an AAV8 vector.

36. The method of claim 33. wherein the Cas nuclease is Cas9.

37. The method of claim 33, wherein the first AAV vector and/or the second AAV vector are administered by intravitreal injection.

38. The method of claim 33, wherein the first AAV vector and/or the second AAV vector are administered by subretinal injection.

39. A method of increasing glycolysis in a neuronal cell in a patient in need thereof, comprising administering a therapeutically effective amount of:
a recombinant adeno-associated viral (AAV) vector encoding an inhibitor of Tsc1, Sirt6, or other metabolic reprogramming agent, or an inhibitor or activator of anabolism, to at least one neuronal cell in the patient.

40. The method of claim 39, wherein the AAV vector is an AAV2 vector.

41. The method of claim 39, wherein the AAV vector is an AAV8 vector.

42. The method of claim 39, wherein the AAV vector is administered by intravitreal injection.

43. The method of claim 39, wherein the AAV vector is administered by subretinal injection.

44. A method of increasing glycolysis in a neuronal cell in a patient in need thereof, comprising administering to the patient a therapeutically effective amount of:
(a) a first recombinant adeno-associated viral (AAV) vector, wherein the first recombinant AAV comprises, (i) a first sequence(s) encoding at least one guide RNA that hybridizes to the endogenous Tsc1 and/or Sirt6 gene in the patient and,
(b) a second recombinant AAV viral vector comprising a nucleic acid sequence encoding a Cas nuclease; wherein the Cas nuclease cleaves the endogenous Tsc1 and/or Sirt6 gene creating a Tsc1 and/or Sirt6 knockout of the endogenous Tsc1 and/or Sirt6 gene in the patient's neuronal cell.

45. The method of claim 44, wherein the first AAV vector and/or the second AAV vector are an AAV2 vector.

46. The method of claim 44, wherein the first AAV vector and/or the second AAV vector are is an AAV8 vector.

47. The method of claim 44, wherein the Cas nuclease is Cas9.

48. The method of claim 44, wherein the first AAV vector and/or the second AAV vector are administered by intravitreal injection.

49. The method of claim 44, wherein the first AAV vector and/or the second AAV vector are administered by subretinal injection.

50. The method of any one of claims 17, 22, 33 or 44, further comprising administering one or more SIRT6 inhibitors selected from the group consisting of fenugreek seed extract, Vitexin (isolated from Hawthorn tree berries), quercetin, naringenin, vitexin, SYN17739303, BAS 13555470, SYN10360754, and BAS00417531.

51. The method of claim 5 or 14, wherein the inhibitor of SIRT6 is selected from the group consisting of fenugreek seed extract. Vitexin (isolated from Hawthorn tree berries), quercetin, naringenin, vitexin, SYN17739303, BAS13555470, SYN10366754, BAS00417531, and a combinations thereof.


SEQUENCE LISTING (9 sequences)

SEQ ID NO: 1 - protein, 9 aa, Unknown
  Description of Unknown ′LAGLIDADG′ family motif peptide
LAGLIDADG

SEQ ID NO: 2 - DNA, 20 bp, Artificial Sequence
  Description of Artificial Sequence Synthetic oligonucleotide
gaggatgtcggtgaattacg

SEQ ID NO: 3 - DNA, 20 bp, Artificial Sequence
  Description of Artificial Sequence Synthetic primer
gctaatgggaacgagaccaa

SEQ ID NO: 4 - DNA, 20 bp, Artificial Sequence
  Description of Artificial Sequence Synthetic primer
acccacctctctcccctaaa

SEQ ID NO: 5 - DNA, 20 bp, Artificial Sequence
  Description of Artificial Sequence Synthetic primer
gcgtccacttctctttcctg

SEQ ID NO: 6 - DNA, 21 bp, Artificial Sequence
  Description of Artificial Sequence Synthetic primer
tgccacgacatcgaccacccg

SEQ ID NO: 7 - DNA, 21 bp, Artificial Sequence
  Description of Artificial Sequence Synthetic primer
gccatccctgccttcccttgg

SEQ ID NO: 8 - DNA, 21 bp, Artificial Sequence
  Description of Artificial Sequence Synthetic primer
ggtcagattccagtgtgtggg

SEQ ID NO: 9 - DNA, 21 bp, Artificial Sequence
  Description of Artificial Sequence Synthetic primer
gtttagctggcccaaatgttg